(19) United States
(12) Patent Application Publication
(10) Pub. No.: US 2024/0173395 A1
(43) Pub. Date: May 30, 2024

(54) ZIKA/DENGUE VACCINE AND APPLICATION THEREOF

(71) Applicant: Institute of Microbiology, Chinese Academy of Sciences, Beijing (CN)

(72) Inventors: Fu GAO, Beijing (CN); Lianpan DAI, Beijing (CN); Jinghua YAN, Beijing (CN); Kun XU, Beijing (CN); Yuxuan HAN, Beijing (CN); Qihui WANG, Beijing (CN); Qingrui HUANG, Beijing (CN); Jinhe LI, Beijing (CN)

(73) Assignee: Institute of Microbiology, Chinese Academy of Sciences, Beijing (CN)

(21) Appl. No.: 17/775,094

(22) PCT Filed: Nov. 9, 2020
(86) PCT No.: PCT/CN2020/127614

§ 371 Date: May 6, 2022

(30) Foreign Application Priority Data
Nov. 7, 2019 (CN) 201911082867.5

(51) Int. Cl. A61K 39/12; A61K 39/00; A61P 31/14; C07K 14/005; C12N 7/00; C12N 15/86

(52) U.S. Cl. CPC A61K 39/12; A61P 31/14; C07K 14/005; C12N 7/00; C12N 15/86; A61K 2039/5254; A61K 2039/53; C12N 2710/10334; C12N 2710/10343; C12N 2770/24122; C12N 2770/24134

(57) ABSTRACT

Provided in the present disclosure are a Zika/dengue vaccine and its application thereof. The present disclosure introduces a mutation into the E-protein FL fusion region of the Zika virus or dengue virus. Antigens with said mutations are unable to bind to antibodies that causes ADE. After immunization with the vaccine of the present disclosure acquired from the said antigens, production of FL epitope-induced antibodies can be prevented, thereby reducing or eliminating the ADE effect.

### TECHNICAL FIELD

The present disclosure relates to the field of biotechnology, specifically, to a field of Zika/dengue vaccine and its application thereof.

### BACKGROUND

Zika virus (ZIKV) is a mosquito-borne virus, belonging to the genus Flavivirus of the family Flaviviridae. The ZIKV outbreak in the Americas in 2015-2016 spreaded to 84 countries around the world, including China. However, no vaccines and drugs are available so far. Although the global incidence of ZIKV infection has now weakened, ZIKV still poses a threat to people living in endemic areas. Therefore, development of a ZIKV vaccine is urgent.

DENV virus (dengue virus, DENV) has four serotypes and is also a mosquito-borne virus belonging to the genus Flavivirus of the family Flaviviridae.

The structures of ZIKV virus and DENV virus are relatively similar, both of which are icosahedral spherical structures with an envelope. The surface of the envelope contains an envelope (Envelope, E) protein. The internal viral genome is a single-stranded positive-stranded RNA, about 11 kb in length, with only one open reading frame. The translated polyprotein can be cleaved into 3 structural proteins (C, prM, and E) and 7 non-structural proteins (NS1, NS2A, NS2B, NS3, NS4A, NS4B, and NS5).

The E protein is about 53 kD in size and is the main protein on the surface of ZIKV and DENV, which mediates entry of a virus into cells via membrane fusion. Thus, it is an important target for activating neutralizing antibodies. At the same time, E protein is also an important target protein when designing vaccines. The E protein has 504 amino acids and exists as a dimer. Each monomer has three domains, DI, DII, and DIII, respectively. The head of DII (98-109 amino acids) contains a highly conserved fusion loop (FL), in which the sequences of the FL region in both ZIKV virus and DENV virus are completely identical, being D98-R99-G100-W101-G102-N103-G104-C105-G106-L107-F108-G109. The FL region plays a key role in the membrane fusion process of virus invasion. During virus infection, immune cells will produce a large number of antibodies against FL.

The size of prM protein is about 26 kD, which assists in the correct folding of the E protein. The transmembrane region at the 3′ end of prM/E serves as an endoplasmic reticulum retention signal to assist prM and E to form a heterodimer. One of the main functions of the prM protein is to maintain the stability of the E protein. In immature virus particles, the pr polypeptide is located at the tip of the E protein, forming a pr-E spike, which hides the fusion peptide of the E protein. At this time, prM is not easily to be contacted with and cleaved by furin due to steric hindrance. After that, the acidic environment in the Golgi body induces a rearrangement reaction that exposes the cleavage site of furin, and the prM protein is cleaved by furin into the M protein. At this time, the cleaved pr polypeptide is not immediately dissociated from the virion. Instead, the pr polypeptide needs to be exposed to a neutral pH cellular environment before it is released and presents a mature virion.

Since the genetic composition and antigenic properties of ZIKV are very similar to those of the four serotypes of dengue virus (DENV) with amino acid similarity of about 56%, the antibodies induced by ZIKV infection will have strong cross-reactivity to DENV. This is also a factor to be considered in the issue of vaccine safety. There are substantial evidences showing that pre-existing antibodies following ZIKV infection can enhance a subsequent DENV infection due to cross-reactivity with DENV (Fowler et al., 2018: George et al., 2017: Li et al., 2017: Richner et al., 2017; Stettler et al., 2016; Valiant et al., 2018). This phenomenon is called antibody- dependent enhancement (ADE). ADE refers to an antibody enhances viral infection when the antibody is insufficient to neutralize the virus or at a sub-neutralizing concentration (Beltramello et al., 2010; Dejnirattisai et al., 2010). Although epidemiological investigations are still insufficient, pre-existing ZIKV antibodies from human beings, monkeys, and mice have all been shown to enhance DENV infection in cellular experiments (George et al., 2017: Richner et al., 2017; Stettler et al., 2016; Valiant et al., 2018). In addition, it is confirmed in monkey and mouse models that symptoms of DENV infection can be exacerbated by antibodies obtained from ZIKV infection, from vaccine immunization, or by a fetus from the mother (Fowler et al., 2018: George et al., 2017: Richner et al., 2017: Stettler et al., 2016). Therefore, it should be considered that ZIKV vaccine may have an ADE effect on future DENV infection after immunization during ZIKV vaccine design.

Antibodies that elicit ADE responses are mainly induced by the FL fusion region of the virus (Beltramello et al., 2010; Dejnirattisai et al., 2010). In Flavivirus infections, such antibodies account for a large proportion of the total induced antibodies. These antibodies often cross-react between different serotypes due to highly conserved epitopes. Most of the antibodies also have low neutralizing activity, which easily lead to ADE reaction. However, most of the antibodies with high neutralizing activity bind to other epitopes of the E protein. A series of ZIKV neutralizing monoclonal antibodies targeting Domain I (DI), Domain II (DII) and Domain III (DIII) or quaternary epitopes of the E protein have been identified (Barba-Spaeth et al., 2016: Stettler et al., 2016; Wang et al., 2017: Wang et al., 2016; Zhao et al., 2016). Therefore, an ideal ZIKV vaccine design strategy is to transfer the hot spot epitopes of the immune response from the FL region to other neutralizing epitopes.

Antibodies are absorbed by cells through binding to virions and then binding to the Fc γ receptor protein on the surface of myeloid cells, which subsequently promote viral infection. Since DENV has four serotypes, ADE is likely to occur when someone is infected with DENV a second time with a different serotype, which explains the more severe disease phenomenon in human beings after DENV infection (Katzelnick et al., 2017). ADE is used to explain the application limitations of the only currently approved DENV vaccine, Dengvaxia®, which is recommended only for use in DENV seropositive individuals, while an injection of the vaccine can actually exacerbate the risk of dengue infection for seronegative individuals (Rey et al., 2018: Slon-Campos et al., 2019). Therefore, it is also a challenge to avoid ADE during DENV vaccine development.

The disclosure in the background is merely to enhance the understanding of the general background and should not be perceived as an acknowledgement or any form of indication that the disclosure forms the prior art known to those of ordinary skill in the art.

### SUMMARY

The application aims to provide a Zika/dengue vaccine and its application to avoid ADE effect.

The present application has obtained the epitope information of an antibody that causes ADE effect using crystal structure analysis and other structural and functional analysis. The present application provides antigens, for which some mutations are introduced into the E-protein FL fusion region of either a Zika virus or a dengue virus. Antigens with said mutations are unable to bind to antibodies that causes ADE (FLE antibody). One embodiment of the present application also provides a vaccine, which can avoid the production of antibodies induced by the FL epitope after immunization, thereby reducing or eliminating the ADE effect.

In order to achieve the purpose, the examples provides an antigen, having an E protein FL fusion region of Zika virus or dengue virus, wherein the E protein FL fusion region comprises one of the following mutations:
(1) one or two of D98 and N103 site mutations in combination with a three-site mutation of G106, L107 and F108:
(2) one of G106, L107, and F108 site mutations or their combinations: and
(3) a single-site mutation of W101.

(1) one or two of D98 and N103 site mutations in combination with a three-site mutation of G106, L107 and F108 is any one of the following: a five-site mutation of D98, N103, G106, L107, and F108: a four-site mutation of D98, G106, L107, and F108; and a four-site mutation of N103, G106, L107, and F108.

(2) one of G106, L107, and F108 site mutations or their combinations is any one of the following: a single-site mutation selected from the group consisting of G106, L107, and F108 site mutations: a double-site mutation selected from any two of G106, L107, and F108 site mutations: a three-site mutation of G106, L107, and F108.
D98 site mutation refers to the substitution of an aspartic acid (D) at position 98 of E protein with any amino acid except aspartic acid:
N103 site mutation refers to the substitution of an asparagine (N) at position 103 of E protein with any amino acid except asparagine:
G106 site mutation refers to the substitution of a glycine (G) at position 106 of the E protein with any amino acid except glycine:
L107 site mutation refers to the substitution of leucine (L) at position 107 of protein E with any amino acid except leucine:
F108 site mutation refers to the substitution of phenylalanine (F) at position 108 of protein E with any amino acid except phenylalanine:
W101 site mutation refers to the substitution of tryptophan (W) at position 101 of E protein with any amino acid except tryptophan.

D98 site, W101 site, N103 site, G106 site, L107 site or F108 site is located in the E protein FL fusion region. The FL (fusion region) sequence of genus Flavivirus is highly conservative, and the FL sequences of ZIKV virus and DENV virus are completely identical as D98-R99-G100-W101-G102-N103-G104-C105-G106-L107-F108-G109. In one aspect, the numbering of D98, W101, N103, G106, L107 and F108 sites refers to the position in the E protein sequences of Zika virus and dengue virus. Specifically, examples can be referred to the 98, 101, 103, 106, 107 and 108 sites of E protein of the Zika virus shown in SEQ ID NO. 1 (e.g. ZIKV FSS13025 strain, GenBank: JN860885.1).

In a possible embodiment of the above antigen,
the mutation of the E protein FL fusion region is a five-site mutation of D98, N103, G106, L107 and F108;
or, the mutation in the E protein FL fusion region is a three-site mutation of G106, L107 and F108;
or, the mutation in the E protein FL fusion region is a double-site mutation of G106 and L107;
or, the mutation in the E protein FL fusion region is a double-site mutation of G106 and F108;
or, the mutation in the E protein FL fusion region is a double-site mutation of L107 and F108;
or, the mutation in the E protein FL fusion region is a single-site mutation of G106;
or, the mutation in the E protein FL fusion region is a single-site mutation of L107;
or, the mutation in the E protein FL fusion region is a single-site mutation of F108;
or, the mutation in the E protein FL fusion region is a single-site mutation of W101.

In a possible embodiment of the above antigen, the mutation of the E protein FL fusion region is selected from any one or a combination of the following groups consisting of different mutation forms:

| Mutation site | Mutation form |
|---|---|
| Five-site mutations of | D98N/N103T/G106F/L107E/F108W |
| D98/N103/G106/L107/F108 | D98N/N103T/G106F/L107K/F108W |
| | D98N/N103T/G106L/L107E/F108W |
| Three site mutations | G106F/L107E/F108W |
| of G106/L107/F108 | G106F/L107K/F108W |
| | G106L/L107E/F108W |
| Double site mutations | G106L/L107E |
| of G106/L107 | G106L/L107K |
| | G106F/L107E |
| | G106F/L107K |
| | G106F/L107R |
| Double site mutations | G106F/F108W |
| of G106/F108 | G106L/F108W |
| | G106F/F108H |
| | G106Y/F108W |
| | G106W/F108Y |
| Double site mutations | L107E/F108W |
| of L107/F108 | L107K/F108W |
| | L107R/F108W |
| | L107D/F108W |
| | L107K/F108Y |
| Single site mutation | G106L |
| of G106 | G106F |
| | G106W |
| | G106Y |
| | G106I |
| Single site mutation | L107E |
| of L107 | L107K |
| | L107R |
| | L107D |
| | L107T |
| Single site mutation | F108W |
| of F108 | F108H |
| | F108Y |
| | F108P |
| | F108A |
| Single site mutation of W101 | W101A |
| | W101R |
| | W101N |
| | W101D |
| | W101C |
| | W101Q |
| | W101E |
| | W101G |
| | W101H |
| | W101I |
| | W10IL |
| | W101K |
| | W101M |
| | W101F |
| | W101P |
| | W101S |
| | W101T |
| | W101Y |
| | W101V |

In the above table,
D98N mutation refers to the substitution of aspartic acid (D) at position 98 of E protein with asparagine (N).
N103T mutation refers to the substitution of asparagine (N) at position 103 of E protein with threonine (T).
G106F mutation refers to the substitution of glycine (G) at position 106 of E protein with phenylalanine (F);
G106L mutation refers to the substitution of glycine (G) at position 106 of E protein with leucine (L).
L107E mutation refers to the substitution of leucine (L) at position 107 of E protein with glutamic acid (E);
L107K mutation refers to the substitution of leucine (L) at position 107 of E protein with lysine (K).
F108W mutation refers to the substitution of phenylalanine (F) at position 108 of the E protein with tryptophan (W).

Mutations of amino acids are deduced for the rest, and the type of the amino acid represented by a single letter is the general understanding of those skilled in the art.

In a possible embodiment, the antigen comprises the E protein FL fusion region of Zika virus, the antigen further comprises a full sequence or a partial sequence of M protein of Zika virus; preferably, the antigen further comprises a full sequence of M protein of Zika virus;
when the antigen comprises the E protein FL fusion region of dengue virus, the antigen further comprises a full sequence or a partial sequence of M protein of dengue virus; preferably, the antigen further comprises a full sequence of M protein of dengue virus.

M protein is formed when prM structural protein is cleaved by furin. The full sequence or the partial sequence of M protein refers to 0.5%-100%, 50-100%, 60-100%, 70-100%, 80-100%, 90-100% sequence similarity of the M protein, the sequence can be either a sequence selected continuously from the M protein or a combination of fragments selected separately from the M protein.

In a possible embodiment, the antigen comprises the E protein FL fusion region of Zika virus, the antigen further comprises a full sequence or a partial sequence of prM protein of Zika virus; preferably, the antigen further comprises a full sequence of prM protein of Zika virus;
when the antigen comprises the E protein FL fusion region of dengue virus, the antigen further comprises a full sequence or a partial sequence of prM protein of dengue virus; preferably, the antigen further comprises a full sequence of prM protein of dengue virus.

The prM protein is a structural protein of Zika virus or dengue virus, with a size of about 26 kD, and is used for assisting in the correct folding of the E protein. The full sequence or the partial sequence of prM protein refers to 0.5%-100%, 50-100%, 60-100%, 70-100%, 80-100%, 90-100% sequence similarity of the prM protein, the sequence can be either a sequence selected continuously from the prM protein or a combination of fragments selected separately from the prM protein.

In a possible embodiment, the antigen comprises the E protein FL fusion region of Zika virus, the antigen further comprises a full sequence or a partial sequence of E protein of Zika virus; preferably, the antigen further comprises a full sequence of E protein of Zika virus;
when the antigen comprises the E protein FL fusion region of dengue virus, the antigen further comprises a full sequence or a partial sequence of E protein of dengue virus; preferably, the antigen further comprises a full sequence of E protein of dengue virus.

The full sequence or the partial sequence of E protein refers to 0.5%-100%, 50-100%, 60-100%, 70-100%, 80-100%, 90-100% sequence similarity of the E protein, the sequence can be either a sequence selected continuously from the E protein, or a combination of fragments selected separately from the E protein.

The E protein, prM protein, and M protein sequences of Zika virus can be obtained according to the full sequences of Zika virus strain disclosed in NCBI and the prior art. The E protein, prM protein, M protein sequences of dengue virus can be obtained according to the full sequences of four serotypes of dengue virus strain disclosed in NCBI and the prior art.

In a possible embodiment of the above antigen, the Zika virus includes all Zika virus strains, such as ZIKV FSS13025 strain (GenBank: JN860885.1) and ZIKK SMGC-1 strain.

In a possible embodiment of the above antigen, the dengue virus includes four serotypes of dengue virus strains, such as DENV1 (Hawaii strain, GenBank: KM204119), DENV2 (New Guinea C strain, GenBank: KM204118.1), DENV3 (YN02 strain, GenBank: KF824903) and DENV4 (B5 strain, Guangzhou, China, GenBank: AF289029).

One embodiment of the present disclosure also provides an antigen binding epitope of E protein FL fusion region of Zika virus, wherein the E protein FL fusion region of Zika virus is the amino acid sequence of

D98-R99-G100-W101-G102-N103-G104-C105-G106-L107-F108-G109, which comprises one of the following mutations:
(1) one or two of D98 and N103 site mutations in combination with a three-site mutation of G106, L107 and F108;
(2) one of G106, L107, and F108 site mutations or their combinations; and
(3) a single-site mutation of W101.

One embodiment of the present disclosure also provides a Zika virus antigen comprising the above-mentioned antigen binding epitope.

In a possible embodiment, the above-mentioned Zika virus antigen also comprises one or more of the following sequences:
a full sequence or a partial sequence of Zika virus E protein;
a full sequence or a partial sequence of Zika virus M protein;
a full sequence or a partial sequence of Zika virus prM protein.

One embodiment of the present disclosure also provides an antigen binding epitope of E protein FL fusion region of dengue virus, wherein the E protein FL fusion region of dengue virus is the amino acid sequence of D98-R99-G100-W101-G102-N103-G104-C105-G106-L107-F108-G109, which comprises one of the following mutations:
(1) one or two of D98 and N103 site mutations in combination with a three-site mutation of G106, L107 and F108;
(2) one of G106, L107, and F108 site mutations or their combinations; and
(3) a single-site mutation of W101.

One embodiment of the present disclosure also provides a dengue virus antigen comprising the above-mentioned antigen binding epitope.

In a possible embodiment, the above-mentioned dengue virus antigen also comprises one or more of the following sequences:
a full sequence or a partial sequence of dengue virus E protein;
a full sequence or a partial sequence of dengue virus M protein;
a full sequence or a partial sequence of dengue virus prM protein.

One embodiment of the present disclosure also provides an antibody that obtained from the above-mentioned antigen, the above-mentioned Zika virus antigen, and the above-mentioned dengue virus antigen.

One embodiment of the present disclosure also provides a polynucleotide encoding the above-mentioned antigen, the above-mentioned antigen binding epitope, the above-mentioned Zika virus antigen, and the above-mentioned dengue virus antigen.

One embodiment of the present disclosure also provides an expression cassette, recombinant vector, transgenic cell line, recombinant bacteria, adenovirus, lentivirus or viral particle comprising the above-mentioned polynucleotide.

One embodiment of the present disclosure also provides an mRNA encoding the above-mentioned antigen, the above-mentioned antigen binding epitope, the above-mentioned Zika virus antigen, and the above-mentioned dengue virus antigen.

One embodiment of the present disclosure also provides a vaccine, that comprises the above-mentioned antigen, the above-mentioned Zika virus antigen, the above-mentioned dengue virus antigen, the above-mentioned polynucleotide, the above-mentioned expression cassette, recombinant vector, transgenic cell line, recombinant bacteria, adenovirus viruses, lentiviruses or virus particles, or the above-mentioned mRNA as active ingredients.

In a possible embodiment of the above vaccine, the vaccine is one or more of an inactivated vaccine, an attenuated vaccine, a DNA vaccine, an mRNA vaccine, an adenovirus vaccine, other viral vector vaccines, a subunit vaccine or viral particles.

In a possible embodiment of the above vaccine, the vaccine is an adenovirus vaccine.

In a possible embodiment of the above vaccine, the vaccine further comprises a pharmaceutically or veterinarily acceptable vehicle, diluent, adjuvant or excipient.

One embodiment of the present disclosure also provides use of the above-mentioned antigens, the above-mentioned antigen binding epitopes, the above-mentioned antibodies, the above-mentioned polynucleotides, the above-mentioned expression cassettes, recombinant vectors, transgenic cell lines, recombinant bacteria, adenoviruses, lentiviruses or virus particles, or the above-mentioned mRNAs in the manufacture of a vaccine for preventing and/or treating infections of viruses of genus Flavivirus.

One embodiment of the present disclosure also provides use of the above-mentioned antigens, the above-mentioned antigen binding epitopes, the above-mentioned antibodies, the above-mentioned polynucleotides, the above-mentioned expression cassettes, recombinant vectors, transgenic cell lines, recombinant bacteria, adenoviruses, lentiviruses or virus particles, or the above-mentioned mRNAs in the manufacture of detection reagents or kits for detecting infections of viruses of genus Flavivirus.

### Beneficial Effects

(1) Firstly, the present application has obtained the epitope information of an antibody that causes ADE effect based on crystal structure analysis and other structural and functional analysis. For the antigen provided in the examples of the present application, one of the following mutations is introduced into the E protein FL fusion region of Zika virus or dengue virus: i. one or two of D98 and N103 site mutations in combination with a three-site mutation of G106, L107 and F108; ii. one of G106, L107, and F108 site mutations or their combinations; iii. a single-site mutation of W101.

Antigens with said mutations are unable to bind to antibodies that causes ADE (FLE antibody). One embodiment of the present application also provides a vaccine, which can avoid the production of antibodies induced by the FL epitope after immunization, thereby reducing or eliminating the ADE effect.

(2) Secondly, the antigens provided in the examples of the present application cannot bind to the antibody (FLE antibody) that causes ADE. However, the binding ability of the antigen to antibodies targeting other epitopes is not affected. The present application provides examples of several adenovirus vaccines of Zika virus obtained from said antigens, and proves that the obtained recombinant adenovirus vaccine does not reduce the immunogenicity of the antigen, and can still activate the neutralizing antibodies. Moreover, adenovirus vaccines of Zika virus plays a role of protection in virus challenge assay of mice; it can well protect mice against viremia and infection of tissues and organs, and reduce or even eliminate the ADE effect of four serotypes of DENV virus after immunization.

Also, the prevent application proves that the mutated E protein maintains its dimer form with only the amino acid side chain of FL has been changed, which does not change the epitopes of other neutralizing antibodies. Single-cell sequencing of germinal center (GC) B cells is used to analyze the antibody responses induced by recombinant adenovirus vaccine in mice, which demonstrates that recombinant adenovirus vaccines with mutated FL regions significantly reduced FL epitope-induced antibodies compared to vaccines with wild-type FL regions, since the dominant epitope of the antigen has been transferred. This well explains the mechanism how the vaccine obtained from the antigen of the present application is able to eliminate the ADE effect.

(3) The present application also takes the expression plasmids of several adenoviruses of dengue virus as an example, and proves that none of the obtained antigens can bind to the antibody (FLE antibody) that causes ADE. This demonstrates that after immunization with the dengue virus vaccine obtained by the antigen of the present application, the production of antibodies induced by the FL epitope can be avoided, thereby reducing or eliminating the ADE effect caused by the subsequent DENV virus infection.

(4) The vaccines obtained from the antigens provided by the present application can be various informs, such as nucleic acid vaccines, mRNA vaccines, adenovirus vector vaccines, other virus vector vaccines, virus-like particles, virus attenuated vaccines or inactivated vaccines based on the antigenic sequences, chimeric vaccines with other backbones, and the like, which can be used to prepare Zika and quadrivalent dengue vaccines that eliminate the effect of ADE.

### BRIEF DESCRIPTION OF THE DRAWINGS

One or more examples are exemplified by the figures in the corresponding drawings, and these exemplified descriptions do not constitute limitations on the examples. The term “exemplified” used herein means “serving as an instance, example, or illustration”. Any examples described herein as “exemplified” is not necessarily to be interpreted as preferred or advantageous over other examples.

FIG. 1 shows the result of neutralization test in mice immunized with wild-type recombinant adenovirus vaccines AdC7-M/E-WT and AdC7-prM/E-WT.

FIG. 2 shows the experimental result of enhancing DENV-infected cells by immunizing mouse serum with wild-type recombinant adenovirus vaccines AdC7-M/E-WT and AdC7-prM/E-WT.

FIG. 3 shows that Z6 antibody has a certain degree of ADE on the cells of four serotypes infected with DENV.

FIG. 4 shows the structure of a complex comprising Fab fragment of Z6 antibody and ZIKV E protein.

FIG. 5 shows the complex structure of different antibodies and E protein of the genus Flavivirus, wherein, A is the complex structure of Z6 antibody and ZIKV E protein, B is the complex structure of 2A10G6 antibody and ZIKV E protein, C is the complex structure of E53 antibody and WNV E protein, and D is an overlap of the structure of the three antibodies and ZIKV E protein.

FIG. 6 shows the amino acid site analysis of ZIKV E protein binding to Z6 antibody, ZIKV E protein binding to 2A10G6 antibody, and WNV E protein binding to E53 antibody.

FIG. 7 shows a phylogenetic tree of genus Flavivirus.

FIG. 8 shows the mutation sites and sequences of ZIKV M/E MutA/B/C mutants.

FIG. 9 shows the results of M/E-MutA/B/C antigen activity detected by flow cytometry.

FIG. 10 shows the construction process of pAdC7-M/E-MutB/MutC recombinant plasmid.

FIG. 11 shows the neutralizing antibody titers of BALB/c mice serum after immunization with ZIKV recombinant adenovirus vaccine.

FIG. 12 shows the neutralizing antibody titers of Ifnar 1^{-/-} mice serum after immunization with ZIKV recombinant adenovirus vaccine.

FIG. 13 shows the results of mortality rate and body weight changes in Ifnar 1^{-/-} mice immunized with ZIKV recombinant adenovirus vaccine after challenge assay.

FIG. 14 shows the experimental result of ZIKV recombinant adenovirus vaccine protecting Ifnar 1^{-/-} mice against viremia caused by ZIKV infection.

FIG. 15 shows the experimental result of ZIKV recombinant adenovirus vaccine protecting Ifnar 1^{-/-} mice against ZIKV-infected tissues and organs after challenge assay.

FIG. 16 shows the result of the change in neutralizing antibody titers of serum of Ifnar 1^{-/-} mice immunized with ZIKV recombinant adenovirus vaccine before and after the challenge assay.

FIG. 17 shows the experiment result of cross-reaction of four serotypes of DENV by immunizing the serum of BALB/c mice with ZIKV recombinant adenovirus vaccine.

FIG. 18 shows the result of ADE experiment on cells infected with four serotypes of DENV by immunizing the serum of BALB/c mice with ZIKV recombinant adenovirus vaccine.

FIG. 19 shows the result of in vivo experiments to detect the effect of ZIKV recombinant adenovirus vaccine on the pathogenicity of infection-enhanced Ifnα/Br^{-/-}Ifnγr^{-/-} mice.

FIG. 20 shows the conditions for sorting GC B cells that bind to ZIKV E protein from lymph node cells of BALB/c mice immunized with ZIKV recombinant adenovirus vaccine by flow cytometry.

FIG. 21 shows the profiling result of GC B cell antibody binding to ZIKV E protein in BALB/c mice immunized with ZIKV recombinant adenovirus vaccine.

FIG. 22 shows the result of paired HV and LV in antibody profile induced by immunization of BALB/c mice with AdC7-M/E-WT vaccine.

FIG. 23 shows the result of paired HV and LV in antibody profiles induced by immunization of BALB/c mice with AdC7-M/E-MutB vaccine.

FIG. 24-1 shows the result of paired HV and LV in the antibody profile induced by immunization of BALB/c mice with AdC7-M/E-MutC vaccine.

FIG. 24-2 shows the result of paired HV and LV in the antibody profile induced by immunization of BALB/c mice with AdC7-M/E-MutC vaccine.

FIG. 25 shows the result of identification of antibodies induced by immunization of BALB/c mice with ZIKV recombinant adenovirus vaccine and the binding ability to different ZIKV E proteins and DENV E proteins.

FIG. 26 shows the experimental result of detecting the antibodies induced in mice immunized with a representative AdC7-M/E-WT vaccine that promote the ADE effect of DENV-infected cells.

FIG. 27 shows the result of comparison of the monoclonal antibody induced by AdC7-M/E-WT vaccine-immunized mice using V locus with reported FLE monoclonal antibody.

FIG. 28-1 shows the result of the comparison of the sequence similarity between the monoclonal antibody induced in mice immunized by AdC7-M/E-WT vaccine and the reported FLE monoclonal antibody.

FIG. 28-2 shows the result of the comparison of the sequence similarity between the monoclonal antibody induced in mice immunized by AdC7-M/E-WT vaccine and the reported FLE monoclonal antibody.

FIG. 29 shows the affinity results of ZIKV sE-WT protein and ZIKV sE-MutC protein with FLE antibody detected by BIAcore.

FIG. 30 shows the affinity results of ZIKV sE-WT protein and ZIKV sE-MutC protein with non-FLE neutralizing antibodies detected by BIAcore.

FIG. 31 shows a complex protein structure of ZIKV sE-MutC and Z3L1 single chain variable fragment (scFv).

FIG. 32 shows the dimer structure of ZIKV sE-MutC protein.

FIG. 33 shows the overlapping comparative analysis of the complex structure of Z3L1/ZIKV SE MutC and Z3L1/ZIKV SE WT (PDB: 5GZN) after.

FIG. 34 shows the comparative analysis of the overlapping FL region of ZIKV sE-MutC and ZIKV SE-WT (PDB: 5JHM).

FIG. 35 shows the comparative analysis of the structure of FLE antibodies (Z6 antibody, 2A10G6 antibody and E53 antibody) binding to Flavivirus E protein, overlapping with the DII complex structure of E peotein. FIG. 35A, B and C is a Z6 antibody and ZIKV sE protein, a 2A10G6 antibody and ZIKV sE protein (PDB: 5JHL), a E53 antibody and WNV sE protein (PDB: 3150), respectively.

FIG. 36 shows the antigenic activity result of DENV2 wild-type and mutant E proteins using flow cytometry.

FIG. 37 shows the antigenic activity result of tryptophan at position 101 of ZIKV E protein to other 19 amino acids using flow cytometry.

### DETAILED DESCRIPTION

In order to more clearly illustrate the objects, technical solutions and advantages of the examples of the present disclosure, the technical solutions in the examples of the present disclosure will be clearly and completely described in the following. It is obvious that the described examples are a part of the examples of the present disclosure, but not all of them. All other examples obtained by an ordinary skilled person in the art based on the examples of the present disclosure without creative efforts shall be within the scope of the present disclosure. Unless specifically stated otherwise, the term “comprising” or variations thereof such as “including” or “having” and the like used throughout the specification and claims will be understood to include the stated element or component, and other elements or other components are not excluded.

In addition, in order to better illustrate the present disclosure, numerous specific details are given in the following detailed description. It will be understood by those skilled in the art that the present disclosure may be implemented without certain specific details. In some examples, materials, elements, methods, means, etc. that are well known to those skilled in the art are not described in detail, so as to highlight the subject of the present disclosure.

### Example 1 Detection of Humoral Immune Responses in Mice Induced by Recombinant Chimpanzee Adenovirus Vaccine Constructed With ZIKV Wild-Type M/E and prM/E Antigens

The M/E antigen of ZIKV FSS13025 virus strain (GenBank: JN860885.1) was constructed into type 7 chimpanzee adenovirus vector. After packaging, culture and purification of the adenovirus, the recombinant adenovirus vaccine AdC7-M/E-WT was obtained (the recombinant adenovirus vaccine AdC7-M/E-WT: Xu et al. (2018) Journal of virology. vol. 92, 6 e01722-17. 26 February as the construction control). Experimental results show that it had a good protective effect on mice. It has been reported in literatures that the adenovirus vaccine constructed with ZIKV prM/E antigen also has protective effect. Therefore, the prM/E antigen of ZIKV-SMGC-1 virus strain was constructed into type 7 chimpanzee adenovirus vector, and the recombinant adenovirus AdC7-prM/E-WT was obtained after packaging (Recombinant adenovirus AdC7-prM/E-WT was constructed according to Hassan, Ahmed O et al. (2019) Cell reports, vol. 28, 10: 2634-2646.e4.), as a control for subsequent experiments.

Evaluation of humoral immune responses in mice induced by recombinant adenovirus vaccines AdC7-M/E-WT and AdC7-prM/E-WT:

15 BALB/c mice were randomly divided into 3 groups and immunized with 1.6×10¹¹ vp (virus particles) of AdC7-M/E-WT adenovirus vaccine, AdC7-prM/E-WT adenovirus vaccine and PBS, respectively. After 4 weeks, the blood was collected. The blood was centrifuged to obtain serum. The serum was inactivated by heating at 56° C. for 30 minutes, and the neutralizing antibody titer of the serum was detected by a micro-neutralization assay.

The process of the micro-neutralization assay was as follows: VERO cells were plated in a 96-well plate one day in advance. The serum was diluted with gradient in 1% FBS (Gibco, 10270-106) in DMEM medium (Invitrogen, C11995500BT) using a 96-well plate the next day. The virus was also diluted with 1% FBS in DMEM medium. The serum and the virus solution were mixed. 100 FFU ZIKV-SMGC-1 was added to each well, and incubated at 37° C. for 2 hours. The supernatant of the medium was removed from the VERO cell plate. A mixture of serum and virus was added, and cultured for 2 hours. After that, DMEM containing 10% FBS was supplemented. The cells were cultured in a 37° C. incubator for 4 days. After 4 days, the cell culture plate was taken out. All supernatant was discarded. The precipitate was washed once with PBS. 150 μl methanol was added for fixation, which was placed in −20° C. refrigerator for 15-20 minutes, and then washed twice with PBS. 2% nonfat milk (blocking solution) in PBS was used for blocking for 30 minutes at room temperature, and then primary antibody was added. The primary antibody was Z6 antibody that binds to ZIKV E protein, diluted to a working concentration of 5 μg/mL with blocking solution, incubated at room temperature for 2 hours, and then washed 3 times with PBST. After that, secondary antibody was added. The secondary antibody was a goat anti-human antibody conjugated to HRP (Proteintech, SA00001-17), which was diluted 1500-fold with blocking solution, incubated at room temperature for 2 hours, and then washed 4 times with PBST. 50 μl of TMB chromogenic solution (Biyuntian, P0209) was added, which was incubated at room temperature, and reacted for about 20 minutes. The color change was monitored, and 50 μl of 2 M hydrochloric acid was added to stop the reaction. The OD450 absorbance value was read on a microplate reader. GraphPad Prism software was used to perform nonlinear fitting on the data to calculate the corresponding serum dilution ratio of neutralizing 50% of the cell infection, as the neutralization titer value (MN₅₀). When the serum at the lowest dilution ratio was still unable to neutralize 50% of the cell infection, the MN₅₀ of the sample was defined as half of the lowest dilution.

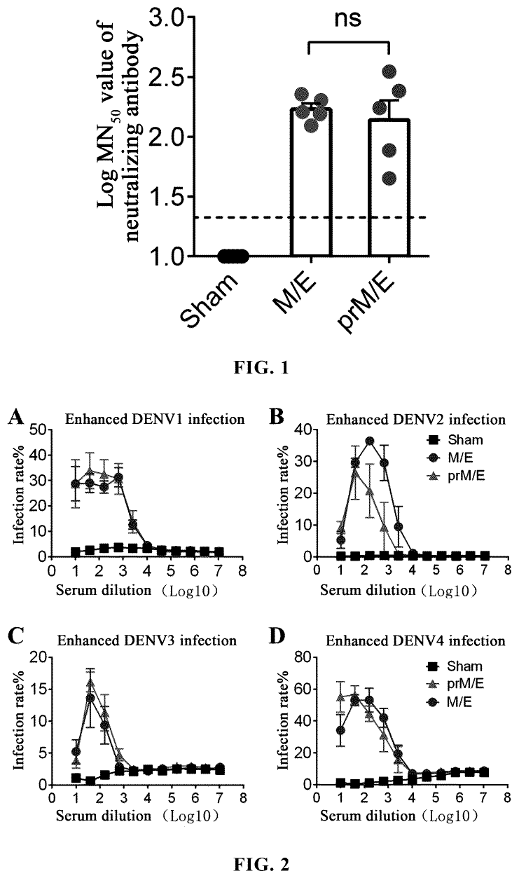

The results of the neutralization assay were shown in FIG. 1. The group immunized with PBS was the negative control group, Sham group (represented by Sham in the figure), and no neutralizing antibodies can be detected in the serum of this group. The log MN₅₀ average value of neutralizing antibodies can be detected in mice in both AdC7-M/E-WT group (indicated by M/E in the figure) and the AdC7-prM/E-WT group (indicated by prM/E in the figure), which were between 2 and 2.5. There was no significant difference between the two groups in statistical analysis, indicating that the recombinant adenovirus vaccines AdC7-M/E-WT and AdC7-prM/E-WT both induce mice to produce relatively high levels of neutralizing antibodies.

### Example 2 In Vitro Experiments to Detect the ADE of DENV in Serums From Mice Immunized With Wild-Type Recombinant Adenovirus Vaccine AdC7-M/E-WT and AdC7-prM/E-WT

Since the fusion loop (FL) sequence of the envelope (E) protein of viruses of genus Flavivirus is highly conserved, infection with ZIKV or immunization with a vaccine expressing the ZIKV E protein can induce the production of antibodies that cross-react with DENV, resulting in an antibody-dependent enhanced response to DENV (ADE) (Stettler, K., et al. (2016) Science: science. aaf8505.). Therefore, it was tested whether the serums from mice immunized with the recombinant adenovirus vaccines AdC7-M/E-WT and AdC7-prM/E-WT could produce ADE against DENV.

Serum (obtained from mice immunized with recombinant adenovirus vaccine in Example 1) was gradiently diluted with RPMI-1640 medium (Invitrogen, C11875500BT) containing 1% FBS. The diluted samples were added to a 96-well plate, 10 μl per well. Then, the corresponding DENV (DENV2, GenBank: KM204118.1; DENV3, GenBank: KF824903; DENV4, GenBank: AF289029; DENV1 was a virus strain that isolated from a sample of an infected patient in Shenzhen Third People's Hospital) was added to each well and incubated in a 37° C. cell incubator for 1 hour.

The cultured K562 cells expressing FcγRIIA receptors on the cell surface were centrifuged at 800 g for 5 minutes, resuspended in RPMI-1640 medium containing 1% FBS, and then counted. After that, the cell density was adjusted to 3×10⁶ cells per ml, added to the mixture comprising virus and serum in 10 μl per well, and incubated in a 37° C. cell incubator for 2 hours. 100 μl of RPMI-1640 medium containing 2% FBS was supplemented to each well, and the incubation was continued in a 37° C. cell incubator for 4 days. After 4 days, the cells were transferred to a 96-well plate, and centrifuged at 800 g for 5 minutes. The supernatant was removed. The precipitate was washed once with PBS, and the cells were collected by centrifugation. 100 μl of Fixation and Permeabilization solution (BD, 554722) was added to each well of the 96-well plate, and placed in a refrigerator at 4° C. for 20 minutes. Then, the cells were harvested by centrifugation at 800 g for 5 minutes and washed twice with 1×Perm/Wash buffer (BD, 554723). 50 μl of FITC-labeled Z6 antibody (Z6-FITC) was added to each well, and placed in a refrigerator at 4° C. for 1 hour. The cells were harvested by centrifugation and washed twice with 1×Perm/Wash buffer. The cells were resuspended in PBS (200 μl per well). The proportion of virus-infected positive cells was detected by flow cytometry.

The results were shown in FIG. 2, K562 cell expressing the FcγRIIA receptor on the cell surface were used as a cell infection model in the experiment. Serums from mice immunized with recombinant adenovirus vaccines in group of AdC7-M/E-WT (indicated by M/E in the figure) and in group of AdC7-prM/E-WT (indicated by prM/E in the figure) showed enhanced effects on DENV-infected K562 cells, and the serum of the control group Sham group (indicated by Sham in the figure) had no ADE. It was speculated that the serums from mice immunized with recombinant adenovirus vaccines AdC7-M/E-WT and AdC7-prM/E-WT have cross-reaction with ZIKV and DENV. Some of the antibodies in the serum can bind to DENV, thereby inducing ADE response.

### Example 3 In Vitro Experiments to Detect the ADE of Monoclonal Antibodies Against DENV1-4

Z6 antibody is a monoclonal antibody binding to ZIKV E protein which was obtained from B cells isolated from the blood of a patient with ZIKV infection by sequencing the antibody sequence, recombinant expressing and purifying. Previous experiments have shown that it mainly bound to the FL epitope of ZIKV E protein (Wang, Qihui, et al. Science translational medicine 8.369(2016):369ra179.). Since the FL sequences of ZIKV and DENV were very conservative and Z6 antibody had low neutralizing activity (Wang, Qihui, et al. Science translational medicine 8.369(2016):369ra179.), it was speculated that the Z6 antibody was likely to cause ADE to DENV, while the use of recombinant chimpanzee adenovirus vaccines AdC7-M/E-WT and AdC7-prM/E-WT constructed with ZIKV wild-type M/E and prM/E antigens were very likely to activate the production of antibodies targeting the FL epitope, thereby leading to ADE.

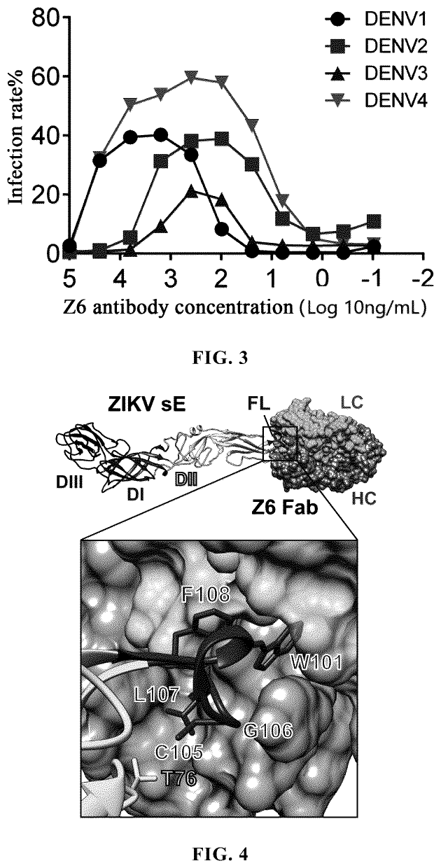

It was tested whether the Z6 antibody will produce an ADE effect to DENV. The results were shown in FIG. 3. The results in FIG. 3 show that the Z6 antibody had a certain degree of ADE against the four serotypes of DENV. The Z6 antibody with lower neutralizing activity cross-reacted with DENV. Presumably, it was likely to cause ADE by binding to the FL epitope of DENV E protein, which will be further studied.

### Example 4 Analysis of the Complex Structure of ZIKV E Protein and Z6 Antibody

Literatures had reported two complex structures of antibodies and antigens of the FL epitope of genus Flavivirus, namely the structure of 2A10G6 antibody and ZIKV soluble E protein (SE) (Dai, Lianpan, et al. Cell Host & Microbe (2016): S1931312816301494.), and the structure of E53 antibody and WNV E protein (Cherier, Mickael V., et al. The EMBO Journal 28.20 (2009): 3269-3276.). 2A10G6 was a Flavivirus broad-spectrum neutralizing antibody that can neutralize DENV1-4, WNV, YFV and ZIKV, which bound to the FL and bc loop of ZIKV E protein (Dai, Lianpan, et al. Cell Host & Microbe (2016): S1931 312 816 301 494.).

In order to further analyze the antibody binding mechanism of ZIKV FL epitope, we obtained the complex structure of the Fab fragment of Z6 antibody and ZIKV E protein with a resolution of 3 Å by combining data analysis with protein crystallization and X-ray diffraction methods. The results were shown in FIG. 4. The data collection and optimized parameters of the complex structures were shown in Table 1.

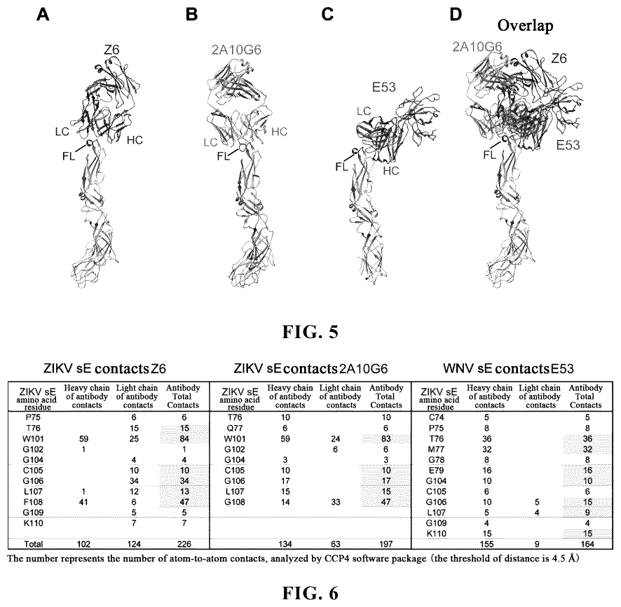

It can be seen from FIG. 4 that, similar to the 2A10G6 antibody, the Z6 antibody bound to the top of DII of the E protein at an almost vertical angle, and interacted with the FL and bc loop of the E protein. However, the difference was that both the heavy and light chains of Z6 interact with the ZIKV E protein, while 2A10G6 focused more on the binding of the heavy chain. The results were shown in FIG. 5. By analyzing the binding amino acid sites, as the results shown in FIG. 6, it was found that the Z6 antibody had more interactions with W101, G106, L107 and F108 of ZIKV E protein. These four amino acid sites also play a key role in binding 2A10G6 antibody. Although the interaction between E53 and E protein was independent of W101, it also has relatively more contacts with G106 and L107.

Therefore, it was predicted that the modification of an immunogen at these 4 amino acid sites can avoid the production of the FL epitope-induced antibody, thereby reducing or eliminating the ADE on DENV.

It had been reported that ZIKV vaccine based on M/E antigen had a good protective effect (Abbink, Peter, et al. Science Translational Medicine 9.420 (2017).). Therefore, we choose M/E antigen for subsequent mutation design.

In addition, it had been reported in the literature that the ZIKV vaccine based on prM/E antigen also had a good protective effect (Dowd et al, Science, 2016, Vol 354, Issue 6309). Our previous data also proved that the mutation design based on M/E antigen had a similar effect with that based on the prM/E antigen. Thus, the experimental results of the mutation design based on the prM/E antigen in the examples of the present disclosure will not be repeated herein. The mutation design for the M/E antigen in the examples of the present disclosure is also applicable to the prM/E antigen.

TABLE 1
Data collection and optimized parameters
for ZIKV sE-Z6 complexes

| Parameter | ZIKV sE-Z6 | |
|---|---|---|
| Data collection | | |
| Space group | C2 | |
| Wavelength (Å) | 0.97853 | |
| Cell parameter | | |
| a, b, c(Å) | 158.25, 153.67, 115.20 | |
| α, β, γ(°) | 90.00, 125.49, 90.00 | |
| Resolution (Å) | 50.00-3.00 | (3.11-3.00) |
| Observed reflection | 238923 | |
| Integrity (%) | 99.8 | (100.0) |
| Redundancy | 11.7 | (11.2) |
| Rpim (%) | 9.4 | (59.6) |
| I/σ | 7.8 | (1.0) |

| | |
|---|---|
| Refinement | |
| Rwork/Rfree(%) | 24.56/27.03 |
| No. atoms | |
| Protein | 12474 |
| Ligand | 0 |
| Water | 0 |
| B-factor | |
| Protein | 80.68 |
| Ligand | |
| Water | |
| r.m.s. deviation | |
| Bond length (Å) | 0.004 |
| Bond angle(°) | 1.038 |
| Ramachandran plot | |
| Favoured(%) | 97.97 |
| Allowed(%) | 2.03 |
| Outliers(%) | 0 |

Values in the parentheses indicate the highest resolution of the shell

### Example 5 Construction of Chimeric Virus Antigen Protein Using the FL of a Virus of Genus Flavivirus With a Large Evolutionary Distance

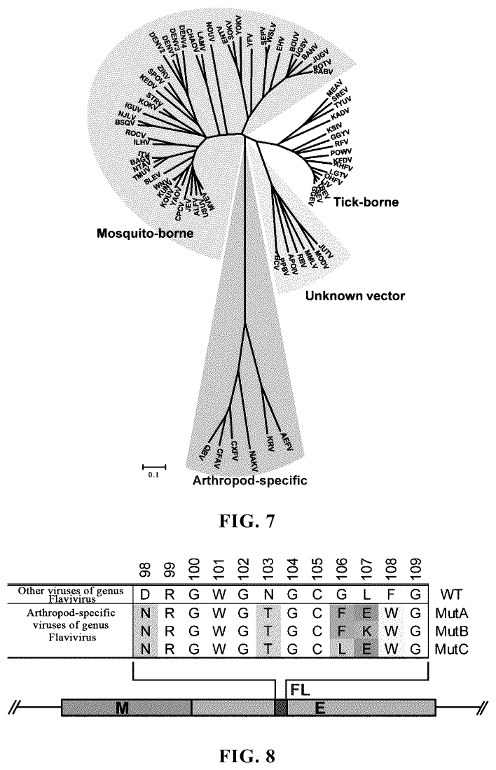

By aligning the amino acid sequences of the E protein FL epitope of viruses of genus Flavivirus, we found that the FL sequences of most viruses were conservative. However, there were still some viral FL sequences with large evolutionary distances, which differ from ZIKV FL sequences. In order to disrupt the ZIKV FL epitope without affecting normal protein folding and display of other neutralizing epitopes, we used the amino acid sequence of the FL of a virus that is evolutionarily distant from ZIKV in the genus Flavivirus (the Flavivirus phylogenetic tree as shown in FIG. 7) to construct a chimeric virus antigen protein. It was speculated that it can better maintain the overall conformation of the antigen and reduce the impact on epitopes other than the mutation site.

We designed mutants of ZIKV M/E antigens with the E protein FL sequences of these viruses as a reference. M and E were full-length, and the mutated site and sequence of the FL fusion region were shown in FIG. 8, in which MutA was derived from AEFV (Aedes Flavivirus, GenBank: KC181923.1), and the mutation sites were D98N, N103T, G106F, L107E and F108W;
MutB was derived from CFAV (Cell fusing agent virus, GenBank: NC_001564.2), and the mutation sites were D98N, N103T, G106F, L107K and F108W;
MutC was derived from NAKV (Nakiwogo virus, GenBank: NC_030400.1), and the mutation sites were D98N, N103T, G106L, L107E and F108W.

The primers used in the construction of the pCAGGS-M/E-MutA/B/C plasmids were shown in Table 2. Taking the construction of MutA as an example, the plasmid pCAGGS-M/E-WT was used as a template, and WT-F and mutA-R were used as primers to obtain the product mutA-1 by PCR. Plasmid pCAGGS-M/E-WT was used as a template, and WT-R and mutA-F were used as primers, to obtain the product mutA-2 by PCR. Then, mutA-1 and mutA-2 were mixed in a molar ratio of 1:1 as a template, and WT-F and WT-R were used as primers for PCR to obtain the PCR product mutA. The pCAGGS plasmid was restricted with XhoI and EcoRI to obtain a linear plasmid with double cohesive ends. The digested linear plasmid and the PCR product mutA were mixed in a molar ratio of 1:5. The In-Fusion kit was used for recombination. The recombinant product was transformed into DH5α competent cells, which was spread on an ampicillin-resistant plate and cultured at 37° C. After that, the clones were picked for PCR identification and sequencing identification, and then the plasmid (pCAGGS-M/E-MutA) was extracted for subsequent experiments.

TABLE 2
The primers used in the construction of pCAGGS-
M/E-MutA/B/C

| Name | |
|---|---|
| of | |
| primer | Primer sequence (5′-3′) |
| WT-F | TTTTGGCAAAGAATTCGCCG (SEQ ID NO. 2) |
| WT-R | GATCTGCTAGCTCGAGTCAAGCGCTCACAGCTGTGGACAGA |
| | (SEQ ID NO. 3) |
| mutA-R | GTGTAAGAGGACCCTGGTGAACAGGGGCTGGGGAACAGGCT |
| | GCTTCGAATGGGGA (SEQ ID NO. 4) |
| mutA-F | GTGAACAGGGGCTGGGGAACAGGCTGCTTCGAATGGGGAAA |
| | GGGCTCCCTGGTG (SEQ ID NO. 5) |
| mutB-R | GTGTAAGAGGACCCTGGTGAACAGGGGCTGGGGAACAGGCT |
| | GCTTCAAGTGGGGA (SEQ ID NO. 6) |
| mutB-F | GTGAACAGGGGCTGGGGAACAGGCTGCTTCAAGTGGGGAAA |
| | GGGCTCCCTGGTG (SEQ ID NO. 7) |
| mutC-R | GTGTAAGAGGACCCTGGTGAACAGGGGCTGGGGAACAGGCT |
| | GCCTGGAATGGGGA (SEQ ID NO. 8) |
| mutC-F | GTGAACAGGGGCTGGGGAACAGGCTGCCTGGAATGGGGAAA |
| | GGGCTCCCTGGTG (SEQ ID NO. 9) |

### Example 6 Detection of M/E-MutA/B/C Antigen Activity

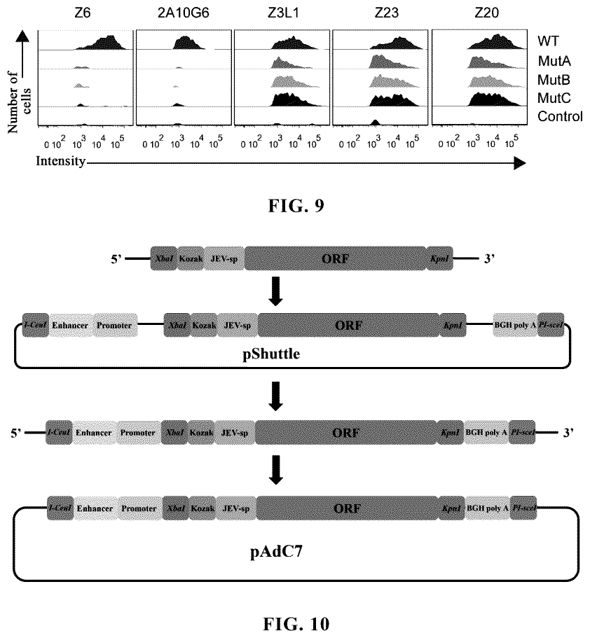

293T cells were transfected with wild-type plasmid pCAGGS-M/E-WT and mutant plasmids pCAGGS-M/E-MutA, pCAGGS-M/E-MutB and pCAGGS-M/E-MutC, respectively. After 48 hours, cells were collected, digested into single cells, fixed and permeabilized, incubated with ZIKV E-binding antibody, incubated with Goat Anti-Human FITC secondary antibody, and finally detected the positive proportion of samples by flow cytometry. The results were shown in FIG. 9.

Z3L1, Z20 and Z23 were ZIKV-specific antibodies with high neutralizing activity, which bind to DI, DII and DIII of ZIKV E protein, respectively (Wang, Qihui, et al. (2016) Science translational medicine 8.369:369 ra179.).

As can be seen from FIG. 9, the antibodies (Z6 and 2A10G6) that bind to the FL epitope of the ZIKV E protein can bind to cells expressing the wild-type M/E-WT antigen, but none of them bound to cells expressing the 3 mutant antigens. The Z3L1, Z23 and Z20 antibodies with high neutralizing activity that bound to non-FL epitopes bind both to cells expressing the wild-type M/E-WT antigen and to cells expressing the 3 mutant antigens, indicating that the FL epitopes on M/E-MutA, M/E-MutB and M/E-MutC antigens were destructed, so that it was impossible for the corresponding antibodies to bind. However, the epitopes bound by other strong ZIKV neutralizing antibodies did not change, and the corresponding antibodies were still able to bind.

That is, M/E-MutA, M/E-MutB and M/E-MutC antigens can induce less or no antibodies that bind to DENV FL, thereby reducing ADE on DENV. At the same time, M/E-MutA, M/E-MutB and M/E-MutC antigens do not affect other antibody epitopes.

### Example 7 Construction of Recombinant Chimpanzee Adenovirus Vaccines AdC7-M/E-MutB and AdC7-M/E-MutC Using M/E-MutB and M/E-MutC Antigens of ZIKV

It can be seen from FIG. 9 that the intensity of the positive cells of MutB and MutC was slightly higher than that of MutA. Therefore, the subsequent experiments were mainly carried out with MutB and MutC.

First, the M/E-MutB and M/E-MutC antigens were cloned into the pshuttle vector. Plasmid pCAGGS-M/E-MutB or pCAGGS-M/E-MutC was used as template, and to_pshuttle-F and to_pshuttle-R were used as primers to carry out PCR reaction to obtain PCR product to_pshuttle-mutB and PCR product to_pshuttle-mutC. The pshuttle plasmid was restricted with XbaI (Thermo, FD0684) and KpnI (Thermo, FD0524) to obtain a linear plasmid with double cohesive ends. The digested linear plasmid was mixed with the PCR product to_pshuttle-mutB or to_pshuttle-mutC in a molar ratio of 1:5. The In-Fusion kit was used for recombination. The recombinant product was transformed into DH5α competent cells, spread on kanamycin-resistant plate, and cultured at 37° C. After that, the clones were picked for PCR identification and sequencing identification, and then the plasmid was extracted. Then the cassettes expressing M/E-MutB and M/E-MutC on pshuttle plasmids were constructed into AdC7 vector. For the above construction steps, see: Xu, Kun et al. (2018) Journal of virology. vol. 92,6 e01722-17. 26 February.

The PCR products to_AdC7-MutB and to_AdC7-MutC were obtained by PCR reaction with plasmid pshuttle-M/E-MutB or MutC as template and to_AdC7-F and to_AdC7-R as primers. The AdC7 plasmid was restricted with PI-SceI (NEB, R0696S) and I-CeuI (NEB, R0699S) to obtain a linear plasmid with double cohesive ends. The digested linear plasmid was mixed with the PCR product to_AdC7-MutB or to_AdC7-MutC in a molar ratio of 1:5. The In-Fusion kit was used for recombination. The recombinant product was transformed into stbl2 competent cells, spread on ampicillin-resistant cells, and cultured at 30° C. After that, the clones were picked for PCR identification and sequencing identification, and then the plasmid was extracted. The construction flow of the recombinant plasmid was shown in FIG. 10. The primer sequences used were shown in Table 3.

TABLE 3
Primers for construction of chimpanzee adenovirus
ZIKV mutant vaccine with MutB and MutC antigens

| Name | |
|---|---|
| of | |
| primer | Primer sequence (5′-3′) |
| to_ | AAACGGGCCCTCTAGAGCCACCATGGGCAAGAGGAGC |
| pshuttle-F | (SEQ ID NO. 10) |
| to_ | TTTAACTTAAGCTTGGTACCTCAAGCGCTCACAGCTG |
| pshuttle-R | TGG (SEQ ID NO. 11) |
| to AdC7-F | GTATAACTATAACGGTCCTAAGGTAGCGAA |
| | (SEQ ID NO. 12) |
| to AdC7-R | TCATTACCTCTTTCTCCGCACCCGACATAG |
| | (SEQ ID NO. 13) |

The pAdC7-M/E-MutB and pAdC7-M/E-MutC plasmids were linearized with PacI (NEB, R0547S) restriction endonuclease, and then heated in a constant temperature bath at 65° C. for 20 min to inactivate the endonuclease. Plasmids were transfected into HEK293 cells using Fugene-6 Transfection Reagent (Promega, E2691), cultured in a 37° C. incubator for at least 7 days, and then checked by microscopy every day for the appearance of plaques. All cells and supernatant were collected as the first-generation recombinant adenovirus when plaque cells fell off. The culture can be scaled up in sequence according to the ratio of 1:10, until the culture reaches 40 plates of cells. All cells were collected, and the cells were lysed by freezing and thawing 3 times to release the virus. After that, cesium chloride was used for density gradient centrifugation. Polyacrylamide gel (Bio-Gel P-6 DG Media, BIO-RAD, 1500738) was used for desalting and purification. OD260 of the sample was detected by NANODROP. The concentration of the sample was the value of OD260 multiplied by 1.1×10¹², with the unit being vp (viral particle)/ml. Aliquots were stored in −80° C.

### Example 8 Evaluation of Humoral Immune Response in BALB/c Mice Induced by Recombinant Adenovirus Vaccines AdC7-M/E-MutB and AdC7-M/E-MutC

24 BALB/c mice were randomly divided into 4 groups and immunized by intramuscular injection with 3 recombinant adenovirus vaccines, namely AdC7-M/E-WT, AdC7-M/E-MutB and AdC7-M/E-MutC. The dose of adenovirus vaccine immunization was 1.6×10¹¹ vp. 1 group of mice was immunized with PBS as a negative control. After 4 weeks, blood was collected to separate serum, and the neutralizing antibody titer in serum was detected by microneutralization assay.

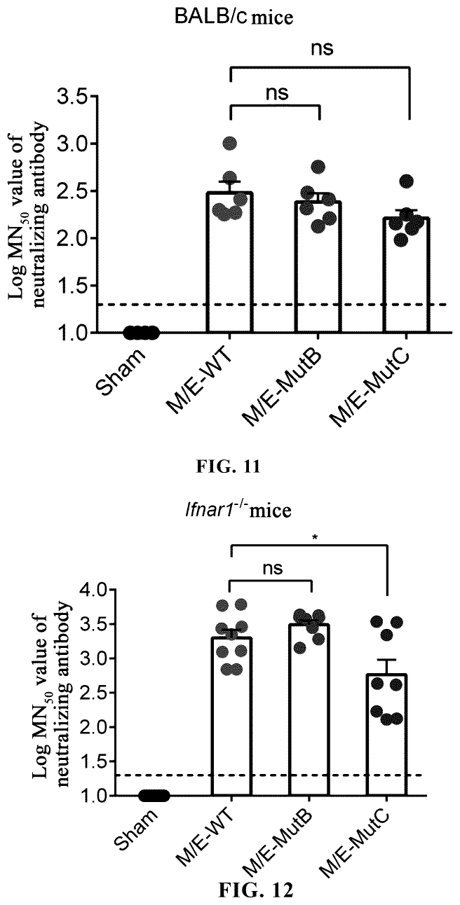

The results were shown in FIG. 11. Both wild-type AdC7-M/E-WT and mutant adenovirus vaccines AdC7-M/E-MutB and AdC7-M/E-MutC can induce neutralizing antibodies in mice. Log(MN₅₀) values ranged from 2.0 to 2.5, and there was no significant difference in neutralizing antibody titers between the AdC7-M/E-MutB and AdC7-M/E-MutC groups and the AdC7-M/E-WT group, indicating that the mutation did not significantly reduce the immunogenicity of the antigen, which could still activate the production of neutralizing antibodies. Among others, as shown in the figure, Sham referred to the PBS-immunized group, M/E-WT referred to the AdC7-M/E-WT vaccine group, M/E-MutB referred to the AdC7-M/E-MutB vaccine group, and M/E-MutC referred to the AdC7-M/E-MutC-immunized vaccine group.

### Example 9 Evaluation of humoral immune response in Ifnar1^{-/-} mice induced by AdC7-M/E-MutB and AdC7-M/E-MutC Vaccines

Since ZIKV infection in BALB/c mice did not cause mouse death and obvious disease symptoms, in order to better verify the effect of the vaccine, we selected the immunodeficient Ifnar 1^{-/-} mouse as the infection model of ZIKV (Lazear, Helen M. et al. (2016), vol. 19,5: 720-30.). Humoral immune responses in Ifnar 1^{-/-} mice induced by AdC7-M/E-MutB and AdC7-M/E-MutC Vaccines were evaluated.

Ifnar 1^{-/-} mice were randomly divided into 4 groups and respectively injected with AdC7-M/E-WT, AdC7-M/E-MutB and AdC7-M/E-MutC adenovirus vaccines by intramuscular injection.

The dose for immunization with the adenovirus vaccine was 1.6×10¹¹ vp, and 1 group of mice was immunized with PBS as a negative control. After 28 days, blood was collected to separate serum, and the titer level of neutralizing ZIKV antibody in the serum of Ifnar 1^{-/-} mice was detected by microneutralization assay. The results were shown in FIG. 12.

As can be seen from FIG. 12, both AdC7-M/E-MutB and AdC7-M/E-MutC vaccines can induce higher levels of neutralizing antibody differences in mice. Among others, as shown in the figure, Sham referred to the PBS-immunized group, M/E-WT referred to the AdC7-M/E-WT vaccine group, M/E-MutB referred to the AdC7-M/E-MutB vaccine group, and M/E-MutC referred to the AdC7-M/E-MutC-immunized vaccine group.

### Example 10 Challenge Protection Experiment After Immunization of Ifnar1^{-/-} mice

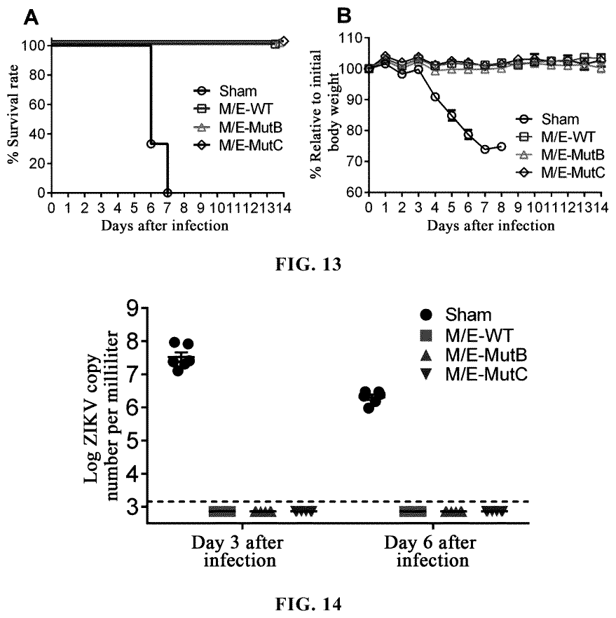

The Ifnar 1^{-/-} mice immunized in Example 9 were challenged with ZIKV virus on Day 30 after immunization, by intraperitoneally injecting and 5×10⁶ PFU ZIKV (SMGC-1 strain). The results were shown in FIG. 13.

It can be seen from FIG. 13 that the body weight of the sham group gradually decreased on Day 4 after the challenge (Panel B in FIG. 13), and all the mice in the sham group died on Day 6 and Day 7 (Panel A in FIG. 13). However, mice immunized with AdC7-M/E-WT, AdC7-M/E-MutB and AdC7-M/E-MutC did not suffer a weight loss after challenge (Panel B in FIG. 13). None of the three groups of mice died (Panel A in FIG. 13). It was indicated that the AdC7-M/E-MutB and AdC7-M/E-MutC vaccines could exhibit a complete protective effect during mice challenge, which was the same as the wild-type AdC7-M/E-WT vaccine. Among others, as shown in the figure, Sham referred to the PBS-immunized group, M/E-WT referred to the AdC7-M/E-WT vaccine group, M/E-MutB referred to the AdC7-M/E-MutB vaccine group, and M/E-MutC referred to the AdC7-M/E-MutC-immunized vaccine group.

To test whether AdC7-M/E-MutB and AdC7-M/E-MutC vaccines can protect mice against viremia induced by viral infection, in this experiment, the blood of mice on Day 3 and Day 6 after the challenge were collected to separated serum. After that, RNA was extracted using MagaBio Plus Viral RNA Kit (Bori Technology, BSC58S1B), and then FastKing One-Step Reverse Transcription-Fluorescence Quantitation Kit (Tiangen Biochemical Technology, FP314) was used for viral RNA Quantification. The probe and the primer sequences used in quantification were shown in Table 4. The quantitative results were shown in FIG. 14.

TABLE 4
Probe and primer sequences used for RT-PCR
quantification of ZIKV-SMGC-1 nucleic acid

| Name | Sequence (5′-3′) |
|---|---|
| ZIKV-probe | FAM - CCACACCTCTGCCGGCACAC - TAMRA |
| | (SEQ ID NO. 14) |
| ZIKV-F | TTGGCTGGCCTATCAGGTTG (SEQ ID NO. 15) |
| ZIKV-R | CACCTCGGTTTGAGCACTCT (SEQ ID NO. 16) |

It can be seen from FIG. 14 that higher viral loads can be detected in the mice in the Sham group on Day 3 and Day 6 after infection, while no virus was detectable in the serums from mice immunized with AdC7-M/E-WT, AdC7-M/E-MutB and AdC7- M/E-MutC adenovirus vaccine on Day 3 and Day 6 after infection, which indicated that AdC7-M/E-MutB and AdC7-M/E-MutC vaccines can protect mice against viremia comparable to that of wild-type AdC7-M/E-WT vaccine. Among others, as shown in the figure, Sham referred to the PBS-immunized group, M/E-WT referred to the AdC7-M/E-WT vaccine group, M/E-MutB referred to the AdC7-M/E-MutB vaccine group, and M/E-MutC referred to the AdC7-M/E-MutC-immunized vaccine group.

### Example 11 Detection of Sterilizing Immune Effect of ZIKV Vaccine

The above experiments demonstrated that immunization of mice with AdC7-M/E-MutB and AdC7-M/E-MutC vaccines could provide protection against viremia and death caused by ZIKV infection. Further experiments were designed to detect whether AdC7-M/E-MutB and AdC7-M/E-MutC vaccines could provide sterilizing immunity. The specific steps were as follows.

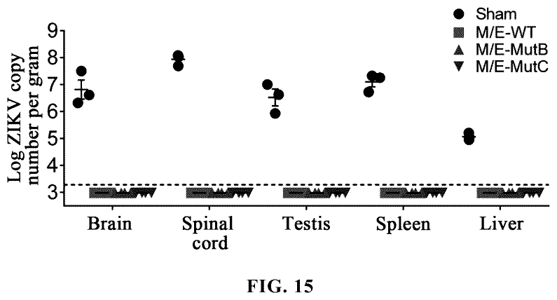

Ifnar 1^{-/-} mice were randomly divided into 4 groups and immunized by intramuscular injection with 3 recombinant adenovirus vaccines, namely AdC7-M/E-WT, AdC7-M/E-MutB and AdC7-M/E-MutC. The dose of adenovirus vaccine immunization was 1.6×10¹¹ vp. 1 group of mice was immunized with PBS as a negative control. On day 28 after immunization, blood was collected to separate serum. On the 30th day after immunization, 5×10¹ FFU of ZIKV (SMGC-1 strain) was intraperitoneally injected. Blood was collected again on day 6 after ZIKV was challenged. After that, parts of liver, spleen, testis, brain and spinal cord were dissected on the same day. The dissected tissues and organs were added to PBS solution, then ground with a grinder (Tiangen Biochemical Technology, OSE-Y30). The supernatant was removed by centrifugation. RNA was extracted using MagaBio Plus virus RNA kit, and then ZIKV RNA was detected by RT-PCR. The results were shown in FIG. 15.

As can be seen from FIG. 15, a certain amount of virus was detected in the 5 organs of the mice in the Sham group, while no virus was detected in all tissues and organs of the 3 groups of mice immunized with AdC7-M/E-WT, AdC7-M/E-MutB and AdC7-M/E-MutC adenoviruses vaccines. Among others, as shown in the figure, Sham referred to the PBS-immunized group, M/E-WT referred to the AdC7-M/E-WT vaccine group, M/E-MutB referred to the AdC7-M/E-MutB vaccine group, and M/E-MutC referred to the AdC7-M/E-MutC-immunized vaccine group.

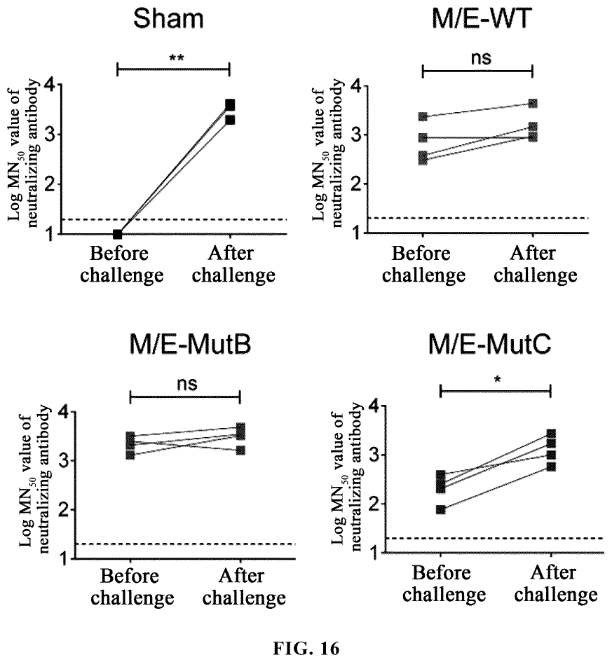

Serum neutralizing antibody titers were detected on day 28 after immunization and on day 7 after challenge using a microneutralization assay. The results were shown in FIG. 16.

As can be seen from FIG. 16, no neutralizing antibody could be detected in the Sham group before the challenge, and a relatively high titer of neutralizing antibody can be detected in each mouse in the Sham group on day 7 after the challenge. The two groups immunized with AdC7-M/E-WT and AdC7-M/E-MutB vaccines had no significant difference in serum neutralizing antibody titers before and after challenge, and maintained at almost the same level, indicating that AdC7-M/E-WT vaccine as well as AdC7-M/E-MutB, and AdC7-M/E-MutC vaccines can provide complete immunity to challenge virus, in which AdC7-M/E-MutB vaccine showing complete sterilizing immunity was slightly better than AdC7-M/E-MutC vaccine. Among others, as shown in the figure, Sham referred to the PBS-immunized group, M/E-WT referred to the AdC7-M/E-WT vaccine group, M/E-MutB referred to the AdC7-M/E-MutB vaccine group, and M/E-MutC referred to the AdC7-M/E-MutC-immunized vaccine group. FIG. 16 sequentially showed the results of the Sham group, the M/E-WT group, the M/E-MutB group, and the M/E-MutC group from left to right.

### Example 12 Detection of Cross-Reaction to DENV in Serum of BALB/c mice Immunized With ZIKV Vaccine

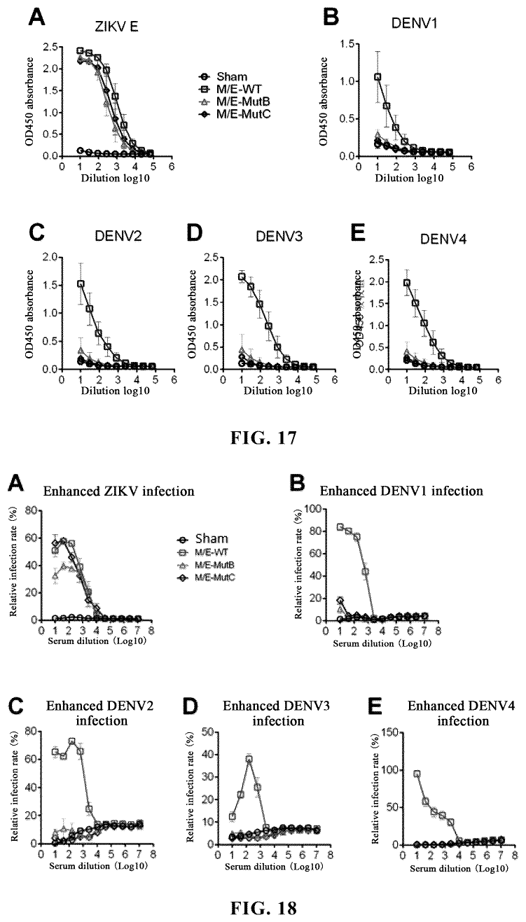

One of the main reasons that ZIKV infection leads to ADE to DENV is that the FL epitope of the E protein of ZIKV and DENV is relatively conservative, so that some antibodies induced by ZIKV that cross-react with DENV (Stettler, K., et al. Science (2016): science.aaf8505.). Therefore, we further detected the cross-reaction between the serum of BALB/c mice immunized with AdC7-M/E-WT, AdC7-M/E-MutB and AdC7-M/E-MutC adenovirus vaccines and the four serotypes of DENV by ELISA experiments. The result was shown in FIG. 17.

It can be seen from FIG. 17 that the serum of BALB/c mice immunized with AdC7-M/E-WT, AdC7-M/E-MutB and AdC7-M/E-MutC vaccine groups had strong binding ability to ZIKV E protein. However, their binding abilities to DENV E protein of four serotypes were different. The serums from mice in M/E-MutB group and M/E-MutC group had very low binding ability to DENV E protein of four serotypes, while the binding abilities of serums from mice in M/E-WT group to the four serotypes of DENV E protein remained at a high level. This indicated that the mutant disrupts the E protein FL epitope and reduced the amount of antibodies induced by this epitope, thus showing a weakened binding ability of the serums from mice immunized with AdC7-M/E-MutB and AdC7-M/E-MutC adenovirus vaccine to bind DENV E protein.

Among others, as shown in the figure, Sham referred to the PBS-immunized group, M/E-WT referred to the AdC7-M/E-WT vaccine group, M/E-MutB referred to the AdC7-M/E-MutB vaccine group, and M/E-MutC referred to the AdC7-M/E-MutC-immunized vaccine group. Panel A in FIG. 17 showed the results of binding ability of mouse serum to Zika virus (ZIKV) E protein. Panel B showed the result of binding ability of mouse serum to serotype 1 dengue virus (DENV1) E protein. Panel C was the result of binding ability between mouse serum and serotype 2 dengue virus (DENV2) E protein. Panel D showed the result of binding ability between mouse serum and serotype 3 dengue virus (DENV3) E protein. Panel E showed the result of binding ability between mouse serum and serotype 4 dengue virus (DENV4) E protein.

### Example 13 In Vitro Experiments to Detect the ADE to DENV of Serum From BALB/c Mice Immunized With ZIKV Vaccine

The experimental results of Example 12 demonstrated that the AdC7-M/E-MutB and AdC7-M/E-MutC vaccines reduced the induction of cross-antibodies against DENV. We further designed experiments to demonstrate whether the AdC7-M/E-MutB and AdC7-M/E-MutC vaccines could reduce ADE to DENV. The specific steps were as follows.

The serums of the immunized BALB/c mice in Example 8 were diluted in gradient, incubated with DENV1, DENV2, DENV3 and DENV4, respectively, and then K562 cells were added. After 4 days of culture, FITC-labeled Z6 antibody was used for staining, followed by flow cytometry to detect the proportion of positive cells. The results were shown in FIG. 18.

DENV virus could not infect K562 cells without antibody mediation. As can be seen from FIG. 18, since the serum of the Sham group did not contain antibodies that could bind to DENV, the infection rate of the detected samples was at the background level. The serums from the mice in the M/E-WT group could mediate four serotypes of DENV virus to infect K562 cells. The infection rates of both groups of the M/E-MutB and M/E-MutC samples were significantly lower than those of the M/E-WT group, showing a weakened or even eliminated ADE to DENV. This indicates that AdC7-M/E-MutB and AdC7-M/E-MutC vaccines achieved a good effect of reducing ADE.

Among others, as shown in the figure, Sham referred to the PBS-immunized group, M/E-WT referred to the AdC7-M/E-WT vaccine group, M/E-MutB referred to the AdC7-M/E-MutB vaccine group, and M/E-MutC referred to the AdC7-M/E-MutC-immunized vaccine group.

In FIG. 18, Panel A shows the detection of enhancement on ZIKV infection of K562 cells by mouse serum. Panel B shows the detection of enhancement on DENV1 infection of K562 cells by mouse serum. Panel C shows the detection of enhancement on DENV2 infection of K562 cells by mouse serum. Panel D shows the detection of enhancement on DENV3 infection of K562 cells by mouse serum. Panel E shows the detection of enhancement on DENV4 infection of K562 cells by mouse serum.

The above series of in vitro experiments had proved that AdC7-M/E-MutB and AdC7-M/E-MutC adenovirus vaccines could reduce or even eliminate the ADE to DENV after immunization.

### Example 14 In Vivo Experiments to Detect the ADE to DENV in Serum From BALB/c Mice Immunized With ZIKV Vaccine

To further demonstrate whether AdC7-M/E-MutB and AdC7-M/E-MutC vaccines can reduce ADE to DENV under physiological conditions, we used a model based on Ifnα/Br^{-/-}Ifnγr^{-/-} mice to validate ADE effect on DENV.

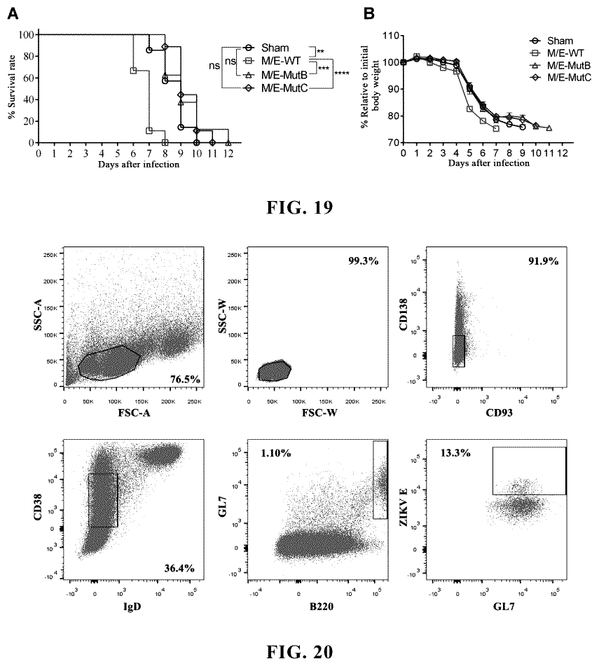

First, 80 BALB/c mice were randomly divided into 4 groups of 20 mice each and immunized with AdC7-M/E-WT, AdC7-M/E-MutB and AdC7-M/E-MutC adenovirus vaccine and PBS, respectively. The dose of adenovirus vaccine immunization was 1.6×10¹¹ vp each mouse. Blood was collected after 4 weeks to separate serum, which was heated at 56° C. for 30 minutes, and the serums from 20 mice in each group were mixed together for subsequent passive immunization of Ifnα/Br^{-/-}Ifnγr^{-/-} mice. Ifnα/Br^{-/-}Ifnγr^{-/-} mice were randomly divided into 4 groups and intraperitoneally injected with serums from BALB/c mice immunized with AdC7-M/E-WT, AdC7-M/E-MutB and AdC7-M/E-MutC adenovirus vaccine or PBS, respectively (serums were diluted with PBS at a ratio of 1:10, and each mouse was injected with 200 μl of the diluted serum). DENV2 virus was injected subcutaneously at 5000 FFU per mouse 24 hours later, and each mouse was weighed before injection. After that, the status, survival and body weight of the mice were observed every day, and the results were shown in FIG. 19.

As can be seen from FIG. 19, compared with the mice in the Sham group, the mice in the M/E-WT group died earlier, indicating that the serum of the mice immunized with the AdC7-M/E-WT vaccine enhanced the incidence of DENV. Additionally, the time and trend of death of mice in the M/E-MutB and M/E-MutC groups were consistent with those in the Sham group (panel A in FIG. 19), indicating that mutations to the ZIKV E protein reduced the production of antibodies that led to ADE effect against DENV, such that the performance of mice in M/E-MutB and M/E-MutC groups were similar to that in the Sham group. The change trend of body weight also showed that the weight of the mice in the M/E-WT group decreased faster, while the change trend of the weight of the mice in the M/E-MutB and M/E-MutC groups was consistent with that of the Sham group (Panel B in FIG. 19). These in vivo results clearly demonstrated that AdC7-M/E-MutB and AdC7-M/E-MutC vaccines could significantly reduce ADE against DENV after immunization of wild-type vaccine.

Among others, as shown in FIG. 19, Sham referred to the PBS-immunized group, M/E-WT referred to the AdC7-M/E-WT vaccine group, M/E-MutB referred to the AdC7-M/E-MutB vaccine group, and M/E-MutC referred to the AdC7-M/E-MutC-immunized vaccine group.

### Example 15 Profile Analysis of Antibodies Induced by ZIKV Vaccine in BALB/c Mice

Since the FL-replaced ZIKV vaccine induced protective immunity while reducing the ADE response to DENV, we further analyzed the B-cell profile of ZIKV E in mice to explain how the mutated vaccine affected antibody responses.

BALB/c mice were randomly divided into 3 groups, and were immunized with AdC7-M/E-WT adenovirus vaccine (WT group), AdC7-M/E-MutB adenovirus vaccine (MutB group) and AdC7-M/E-MutC adenovirus vaccine (MutC group) by intramuscular injection, 1.6×10¹¹ vp per mouse. On day 20 after immunization, the lymph nodes were dissected out and placed in 1640 medium containing 1% FBS. The lymph nodes of all mice in each vaccine group were mixed together, ground using the rough side of a glass slide, and followed by filtration with a 0.45 μm filter. Lymphocytes were centrifuged at 400 g for 15 minutes at 4° C., the supernatant was discarded and the precipitate was resuspended in 1 ml of FACS buffer. FACS buffer was a PBS solution comprising 0.5% FBS. After resuspension, it was transferred to a 1.5 ml EP tube, centrifuged at 400 g for 10 minutes at 4° C. The supernatant was discarded. The cells were resuspended in 200 μl FACS buffer. 4 μg biotin-labeled mixture of ZIKV E monomeric protein and dimeric protein were added, and incubated at 4° C. for 30 min in the dark. Then, 1 ml of FACS buffer was added, mixed well, and centrifuged to precipitate cells. After that, 1 ml of FACS buffer was added to wash again, and then antibody was added for staining. The antibody was diluted with FACS buffer. Each 200 μl of antibody solution contained: FITC-GL7, 2 μl (BD, 553666); PE-CD138, 4 μl (BD, 553714); PE/CY7-CD38, 4 μl (BioLegend, 102718); APC-CD93, 4 μl (BioLegend, 136510); BV421-B220, 16 μl (BioLegend, 103240); BV510-IgD, 2 μl (BD, 563110); BV711, 4 μl (BD, 563262). 200 μl of antibody solution was added to each sample, which was incubated at 4° C. for 30 min in the dark, and then washed twice with FACS buffer. 2 ml of FCAS buffer was added. The cells were resuspended, filtered with a 0.45 μm filter, and transferred to a flow tube. The sorting conditions were GL-7+B220hi CD38lo IgD-CD93-CD138-+(as shown in FIG. 20). GC B cells that reacted with ZIKV E were sorted, which were then used for library construction before sequencing.

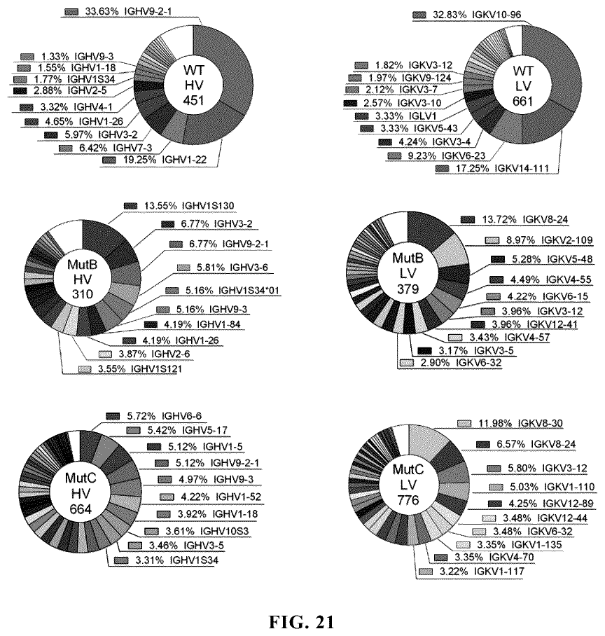

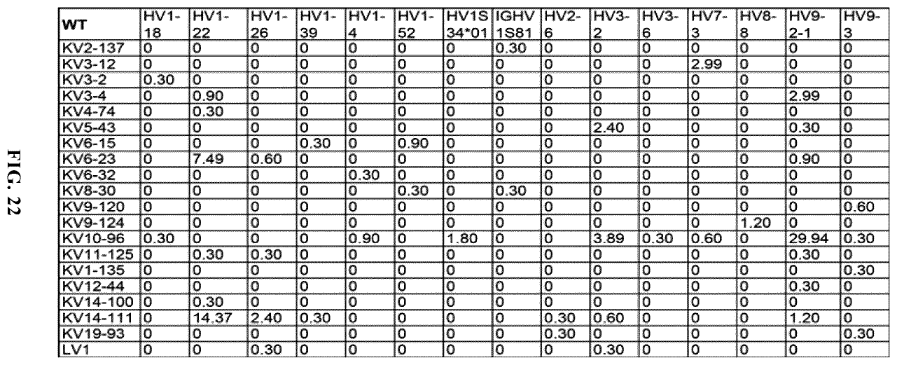

We used single-cell sequencing technology to obtain single-cell paired B cell receptor (BCR) sequences. Chromium Single Cell V(D)J Enrichment Kit, Mouse B Cell, 96 rxns (10×genomics, PN-1000072) kit was used for library construction before sequencing. Then, high-throughput sequencing was performed to analyze the full-length sequence of the V(D)J fragment of light chain and heavy chain in each cell. The results were shown in FIG. 21 and Table 5.

As can be seen from FIG. 21 and Table 5, 451 heavy chain variable region sequences and 661 light chain variable region sequences were obtained from the WT group samples, which could be matched to 334 pairs. 310 heavy chain variable region sequences and 379 light chain variable region sequences were obtained from the MutB group samples, which could be matched to 234 pairs. 664 heavy chain variable region sequences and 776 light chain variable region sequences were obtained from the MutC group samples, which could be matched to 515 pairs. Among others, in FIG. 21 and Table 5, WT referred to the group immunized with AdC7-M/E-WT vaccine, MutB referred to the group immunized with AdC7-M/E-MutB vaccine, and MutC referred to the group immunized with AdC7-M/E-MutC vaccine.

TABLE 5
Summary of BCR sequencing data

| | WT | MutB | MutC |
|---|---|---|---|
| The number of cells entered | ~2500 | ~750 | ~3000 |
| Number of cells in which Ig sequences are detected | 490 | 321 | 621 |
| Average number of read pairs per cell | 72054 | 125947 | 63305 |
| Number of cells with HV:LV pairs | 334 | 234 | 515 |
| Frequency that the representative antibody | 9/10 | 7/8 | 10/13 |
| binds to the E protein as detected | (90.0%) | (87.5%) | (76.9%) |

Analysis of antibody profiles in WT, MutB and MutC-induced mice showed that the AdC7-M/E-WT vaccine activated the variable region (V) gene in mice with a preference, in which about 60% of the heavy chains used IGHV9-2-1, IGHV1-22 and IGHV7-3, and about 60% of the light chains used IGKV10-96, IGKV14-111 and IGKV6-23 (FIG. 21). However, for the FL-substituted AdC7-M/E-MutB and AdC7-M/E-MutC vaccines, the BCR profiles showed more diversity and dispersion in both the heavy chain variable region (HV) and light chain variable region (LV) (FIG. 21). In the WT group, several variable region genes with the most significant activation were significantly reduced or even absent in the antibody profiles of the FL-substituted vaccine groups MutB and MutC groups (FIG. 21).

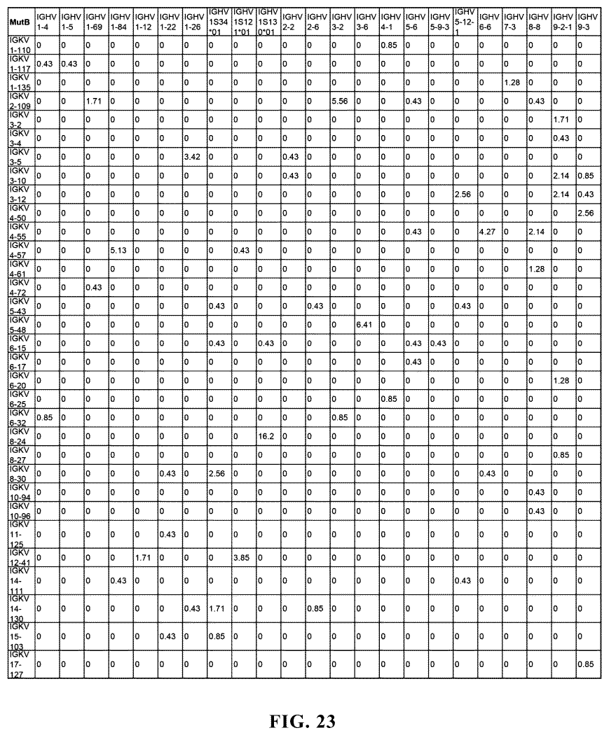

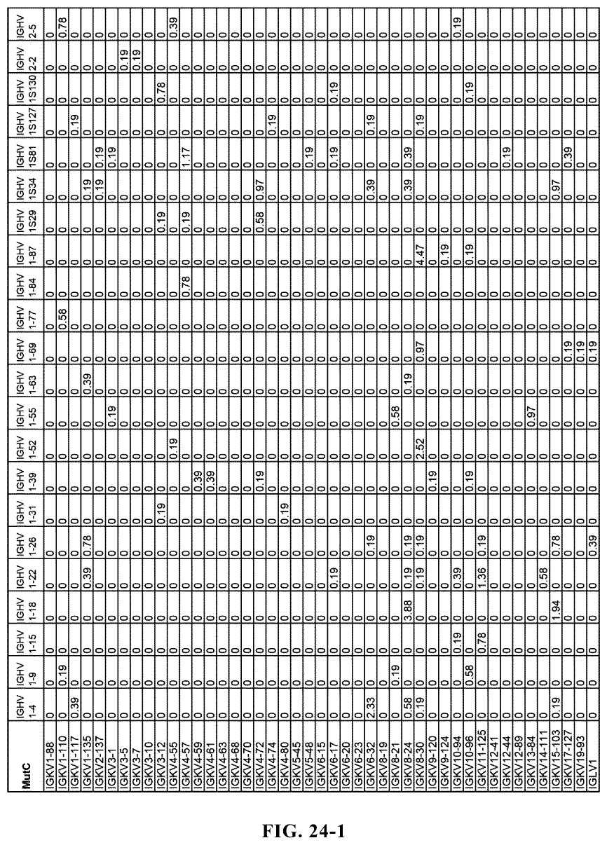

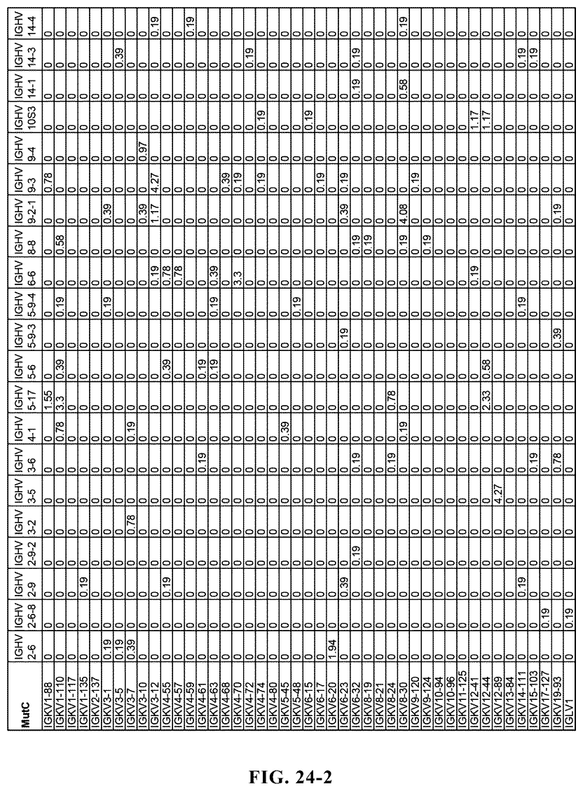

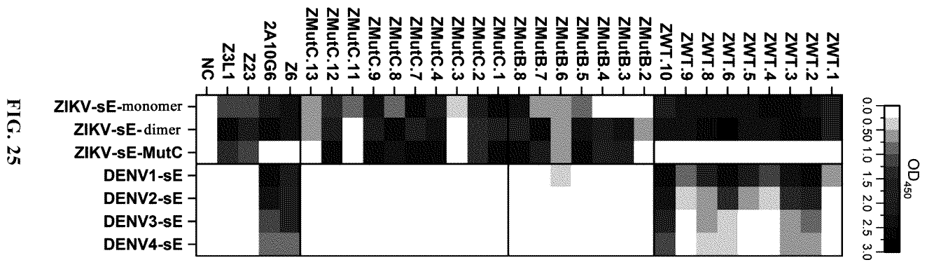

We then analyzed the paired HV and LV results in the antibody profile. The results were shown in FIG. 22, FIG. 23, FIG. 24-1 and FIG. 24-2, respectively.

It can be seen from FIG. 22 that the AdC7-M/E-WT vaccine activated the HV:LV of GC B cell clones in mice with a clear preference, in which IGHV9-2-1:IGKV10-96 (29.9%), IGHV1-22:IGKV14-111 (14.4%) and IGKV1-22:IGKV6-23 (7.5%) had the highest frequency. The sum of these three was about 50%.

As can be seen from FIG. 23 and FIGS. 24-1 and 24-2, HV:LVs of GC B cell clones in mice activated with AdC7-M/E-MutB and AdC7-M/E-MutC vaccines were more diverse and more dispersed in frequency distribution. The highest IGHV9-2-1:IGKV10-96 was detected in the WT group, which was absent in both MutB and MutC groups.

The results of antibody profiling showed that the immunodominant epitopes of the B cell response were transferred after substituting the FL region of the AdC7-M/E-WT vaccine.

### Example 16 Identification of Major FLE Antibody Types With ADE Responsive to DENV Infection

Most of the antibodies that caused the ADE response to DENV target FLEs of the E protein. Additionally, the antibodies induced by ZIKV infection also led to the ADE response to DENV. Therefore, we identified the binding characteristics of the isolated monoantibodies and detected which one bound with FLE and whether it induced the ADE response to DENV.

According to the similarity classification of GC B cell clones, some representative monoclonal antibody genes were synthesized (Jinweizhi, Suzhou), covering 63.38%, 46.57% and 43.88% of the sum totals of AdC7-M/E-WT, AdC7-M/E-MutB and AdC7-M/E-MutC group, respectively (Table 6). The HV and LV genes were subsequently cloned into murine IgG2A and Igk expression vectors, respectively. The monoclonal antibodies derived from the AdC7-M/E-WT vaccine group (represented as M/E-WT in Table 6) were named as ZWT.1-10, and the monoclonal antibodies defived from the AdC7-M/E-MutB vaccine group (represented as M/E-MutB in Table 6) and AdC7-M/E-MutC vaccine group (represented as M/E-MutC in Table 6) were named as ZMutB.1-8 and ZMutC.1-13, respectively (Table 6).

TABLE 6
Expression of murine monoclonal antibodies

| | | | | | | | | | | | Number of | | |
|---|---|---|---|---|---|---|---|---|---|---|---|---|---|
| | | | | | | | | | | | clones per | | Bind- |
| | | | | | | | | | | | group | | ing |
| | | | | | | HV | | | | LV | c [symbol] red | | to |
| mAb | Vaccine | HV | HD | HJ | CDRH3 | simi- | LV | LJ | CDRL3 | simi- | by genetic | Fre- | ZIKV |
| ID | group | gene | gene | gene | length | larity | gene | gene | length | larity | composition | quency | sE^{b} |
| ZWT.1 | M/E- | IGHV9- | IGHD2- | IGHJ | 11 | 98.60% | IGKV10- | IGKJ | 9 | 99.00% | 100 | 29.94% | Yes |
| | WT | 2-1*01 | 1*01 | 4*01 | | | 96*01 | 1*01 | | | | | |
| ZWT.2 | M/E- | IGHV9- | IGHD2- | IGHJ | 11 | 99.30% | IGKV10- | IGKJ | 9 | 99.30% | | | |
| | WT | 2-1*01 | 13*01 | 4*01 | | | 96*01 | 1*01 | | | | | |
| ZWT.3 | M/E- | IGHV9- | IGHD3- | IGHJ | 11 | 95.60% | IGKV10- | IGKJ | 9 | 97.90% | | | |
| | WT | 2-1*01 | 3*01 | 4*01 | | | 96*01 | 1*01 | | | | | |
| ZWT.4 | M/E- | IGHV1- | IGHD1- | IGHJ | 14 | 94.90% | IGKV14- | IGKJ | 9 | 98.60% | 48 | 14.37% | Yes |
| | WT | 22*01 | 1*01 | 2*01 | | | 111*01 | 1*01 | | | | | |
| ZWT.5 | M/E- | IGHV1- | IGHD1- | IGHJ | 11 | 95.20% | IGKV14- | IGKJ | 9 | 98.30% | | | |
| | WT | 22*01 | 1*02 | 4*01 | | | 111*01 | 1*01 | | | | | |
| ZWT.6 | M/E- | IGHV1- | IGHD2- | IGHJ | 16 | 95.90% | IGKV6- | IGKJ | 8 | 97.90% | 25 | 7.40% | Yes |
| | WT | 22*01 | 14*01 | 2*01 | | | 23*01 | 2*01 | | | | | |
| ZWT.7 | M/E- | IGHV3- | IGHD1- | IGHJ | 12 | 99.00% | IGKV10- | IGKJ | 9 | 99.70% | 13 | 3.89% | No |
| | WT | 2*02 | 1*01 | 2*01 | | | 96*01 | 1*01 | | | | | |
| ZWT.8 | M/E- | IGHV7- | IGHD1- | IGHJ | 10 | 97.70% | IGKV3- | IGKJ | 8 | 98.00% | 10 | 2.99% | Yes |
| | WT | 3*02 | 3*01 | 2*01 | | | 12*01 | 1*01 | | | | | |
| ZWT.9 | M/E- | IGHV9- | IGHD2- | IGHJ | 11 | 99.70% | IGKV3- | IGKJ | 8 | 98.00% | 10 | 2.99% | Yes |
| | WT | 2-1*01 | 1*01 | 4*01 | | | 4*01 | 2*01 | | | | | |
| ZWT.10 | M/E- | IGH | N/A | IGHJ | 11 | 97.00% | IGKV10- | IGKJ | 8 | 97.90% | 6 | 1.80% | Yes |
| | WT | V [symbol] S3 | | 4*01 | | | 96*01 | 1*01 | | | | | |
| | | 4*01 | | | | | | | | | | | |
| ZMut | M/E- | IGH | IGHD2- | IGHJ | 14 | 98.30% | IGKV8- | IGKJ | 9 | 99.30% | 38 | 16.23% | No |
| B.1 | Mut | V [symbol] 81 | 2*01 | 1*01 | | | 24*01 | 1*01 | | | | | |
| | B | 30*01 | | | | | | | | | | | |
| ZMut | M/E- | IGHV3- | IGHD1- | IGHJ | 9 | 99.00% | IGKV5- | IGKJ | 9 | 98.60% | 15 | 6.41% | Yes |
| B.2 | Mut | 6*02 | 3*01 | 3*01 | | | 48*01 | 4*01 | | | | | |
| | B | | | | | | | | | | | | |
| ZMut | M/E- | IGHV3- | IGHD5- | IGHJ | 14 | 99.70% | IGKV2-1 | IGKJ | 9 | 98.70% | 13 | 5.56% | Yes |
| B.3 | Mut | 2*02 | 2*01 | 1*01 | | | 09*01 | 5*01 | | | | | |
| | B | | | | | | | | | | | | |
| ZMut | M/E- | IGHV1- | N/A | IGHJ | 7 | 97.60% | IGKV4- | IGKJ | 9 | 97.20% | 12 | 5.12% | Yes |
| B.4 | Mut | 84*02 | | 2*01 | | | 57*01 | 5*01 | | | | | |
| | B | | | | | | | | | | | | |
| ZMut | M/E- | IGHV6- | IGHD3- | IGHJ | 12 | 99.30% | IGKV4- | IGKJ | 9 | 97.60% | 10 | 4.27% | Yes |
| B.5 | Mut | 6*01 | 1*01 | 2*01 | | | 55*01 | 2*01 | | | | | |
| | B | | | | | | | | | | | | |
| ZMut | M/E- | IGH | IGHD1- | IGHJ | 10 | 96.00% | IGKV12- | IGKJ | 8 | 99.60% | 8 | 3.42% | Yes |
| B.6 | Mut | V [symbol] S1 | 2*01 | 2*01 | | | 41*01 | 1*01 | | | | | |
| | B | 21*01 | | | | | | | | | | | |
| Z.Mut | M/E- | IGHV1- | IGHD2- | IGHJ | 17 | 93.50% | IGKV3- | IGKJ | 9 | 99.70% | 8 | 3.42% | Yes |
| B.7 | Mut | 26*01 | 2*01 | 1*01 | | | 5*01 | 4*01 | | | | | |
| | B | | | | | | | | | | | | |
| ZMut | M/E- | IGHV9- | IGHD1- | IGHJ | 14 | 97.60% | IGKV3- | IGKJ | 9 | 98.70% | 5 | 2.14% | Yes |
| B.8 | Mut | 2-1*01 | 1*01 | 3*01 | | | 10*01 | 5*01 | | | | | |
| | B | | | | | | | | | | | | |
| ZMut | M/E- | IGH | N/A | IGHJ | 7 | 90.50% | IGKV8- | IGKJ | 9 | 98.70% | 27 | 5.24% | Yes |
| C.1 | Mut | V [symbol] S1 | | 2*01 | | | 30*01 | 2*01 | | | | | |
| | C | 27*01 | | | | | | | | | | | |
| ZMut | M/E- | IGHV1- | N/A | IGHJ | 7 | 96.90% | IGKV8- | IGKJ | 9 | 99.70% | 23 | 4.47% | Yes |
| C.2 | Mut | 87*01 | | 2*01 | | | 30*01 | 2*01 | | | | | |
| | C | | | | | | | | | | | | |
| ZMut | M/E- | IGHV3- | IGHD6- | IGHJ | 13 | 98.90% | IGKV12- | IGKJ | 9 | 100.00% | 22 | 4.27% | Yes |
| C.3 | Mut | 5*02 | 2*01 | 1*01 | | | 89*01 | 2*01 | | | | | |
| | C | | | | | | | | | | | | |
| ZMut | M/E- | IGHV9- | IGHD2- | IGHD2- | 14 | 99.70% | IGKV3- | IGKJ | 9 | 99.70% | 22 | 4.27% | Yes |
| C.4 | Mut | 3*02 | 2*01 | 7*01 | | | 12*01 | 4*01 | | | | | |
| | C | | | | | | | | | | | | |
| ZMut | M/E- | IGHV3- | IGHD6- | IGHJ | 13 | 99.00% | IGKV12- | IGKJ | 9 | 100.00% | 22 | 4.27% | No |
| C.5 | Mut | 5*02 | 2*01 | 1*01 | | | 89*01 | 2*01 | | | | | |
| | C | | | | | | | | | | | | |
| ZMut | M/E- | IGHV1- | IGHD2- | IGHJ | 13 | 92.20% | IGKV8- | IGKJ | 9 | 99.70% | 20 | 3.88% | No |
| C.6 | Mut | 18*01 | 14*01 | 1*01 | | | 24*01 | 5*01 | | | | | |
| | C | | | | | | | | | | | | |
| ZMut | M/E- | IGHV9- | IGHD4- | IGHJ | 11 | 98.00% | IGKV8- | JGKJ | 9 | 99.30% | 18 | 3.50% | Yes |
| C.7 | Mut | 2-1*01 | 1*01 | 2*01 | | | 30*01 | 5*01 | | | | | |
| | C | | | | | | | | | | | | |
| Z.Mut | M/E- | IGHV6- | IGHD2- | IGHJ | 13 | 98.00% | IGKV4- | IGKJ | 9 | 98.60% | 17 | 3.30% | Yes |
| 0.8 | Mut | 6*03 | 3*01 | 4*01 | | | 70*01 | 5*01 | | | | | |
| | C | | | | | | | | | | | | |
| Z.Mut | ME- | IGHV9- | IGHD2- | IGHD2- | 14 | 99.70% | IGKV3- | IGKJ | 9 | 99.70% | 16 | 3.11% | Yes |
| C.9 | Mut | 3*02 | 2*01 | 7 | | | 12*01 | 4*01 | | | | | |
| | C | | | | | | | | | | | | |
| ZMut | M/E- | IGHV5- | IGHD2- | IGHJ | 13 | 99.00% | IGKV1- | IGKJ | 9 | 99.70% | 15 | 2.91% | No |
| C.10 | Mut | 17*02 | 3*01 | 4*01 | | | 110*01 | 2*01 | | | | | |
| | C | | | | | | | | | | | | |
| ZMut | M/E- | IGHV1- | IGHD2- | IGHJ | 10 | 90.10% | IGKV15- | IGKJ | 9 | 98.60% | 10 | 1.94% | Yes |
| C.11 | Mut | 18*01 | 2*01 | 2*01 | | | 103*01 | 1*01 | | | | | |
| | C | | | | | | | | | | | | |
| ZMut | M/E- | IGHV3- | IGHD4- | IGHJ | 10 | 99.70% | IGKV4- | IGKJ | 9 | 99.30% | 8 | 1.55% | Yes |
| C.12 | Mut | 6*02 | 1*01 | 2*01 | | | 55*01 | 1*01 | | | | | |
| | C | | | | | | | | | | | | |
| ZMut | M/E- | IGHV10S | IGHD2- | IGHJ | 10 | 98.30% | IGKV12- | IGKJ | 9 | 98.60% | 6 | 1.17% | Yes |
| C.13 | Mut | 3*01 | 2*01 | 3*01 | | | 44*01 | 5*01 | | | | | |
| | C | | | | | | | | | | | | |

^{a} Considering 7 elements of genes: the length of HV, HD, HJ gene and CDRH3 of the heavy chain, and the length of LV, LJ and CDRL3 of the light chain, when the clone has 4 or more same elements, it was considered to belong to the same gene cluster.
^{b} ELISA detects the binding ability of each monoclonal antibody to ZIKV E (monomer or dimer). When the OD450 of the sample was higher than 5 times of the value of the negative control, it was considered to be binding, otherwise it was considered to be non-binding. [symbol]
indicates data missing or illegible when filed

We co-transfected 293T cells with plasmids that express heavy chain and light chain of the monoclonal antibody. The supernatant was collected after 3 days. The binding ability of the antibody in the supernatant to ZIKV-E protein was detected by ELISA.

The experimental method of ELISA was as follows: Dilute the protein with ELISA coating solution (sodium carbonate-sodium bicarbonate buffer, pH 9.6) to 3 μg/ml, add 100 μl to each well of 96-well ELISA plate, and leave standing overnight at 4° C. The next day, discard the coating solution, and block the ELISA plate with 5% nonfat milk in PBS, and leave at room temperature for 1 hour. Pour off the blocking solution, add 100 μl of the culture supernatant expressing monoclonal antibody to each well of the ELISA plate, incubate at room temperature for 2 hours, and wash 3 times with PBST. Afterwards, add Goat Anti-Mouse HRP (ab6789) secondary antibody diluted 1:2000 in blocking solution, incubate at room temperature for 1.5 hours, and wash 4 times with PBST. Add 50 μl of TMB chromogenic solution to develop color, add 50 μl of 2M hydrochloric acid after 30 minutes to stop the reaction, and detect the OD450 value on a microplate reader.

The ELISA test results were shown in FIG. 25. Most of the monoclonal antibodies could bind to the monomer or dimer form of the ZIKV sE protein. As can be seen from Table 6, the positive rate of binding reached 90% (9/10) in the ME-WT group, 87.5% in ME-MutB group (7/8), 76.9% in ME-MutC group (10/13), respectively.

Further evaluation of 26 positive monoclonal antibodies that bound to ZIKV sE protein was performed. From FIG. 25, we found that all monoclonal antibodies derived from ME-WT group did not react with ZIKV sE-MutC protein, but cross-reacted with the sE protein of DENV serotypes 1 to 4, indicating that the monoclonal antibody derived from the ME-WT group was a monoclonal antibody that binds to FLE; and the monoclonal antibodies derived from the ME-MutB and ME-MutC groups were mainly ZIKV-specific antibodies, and they were almost none of them bound to DENV sE.

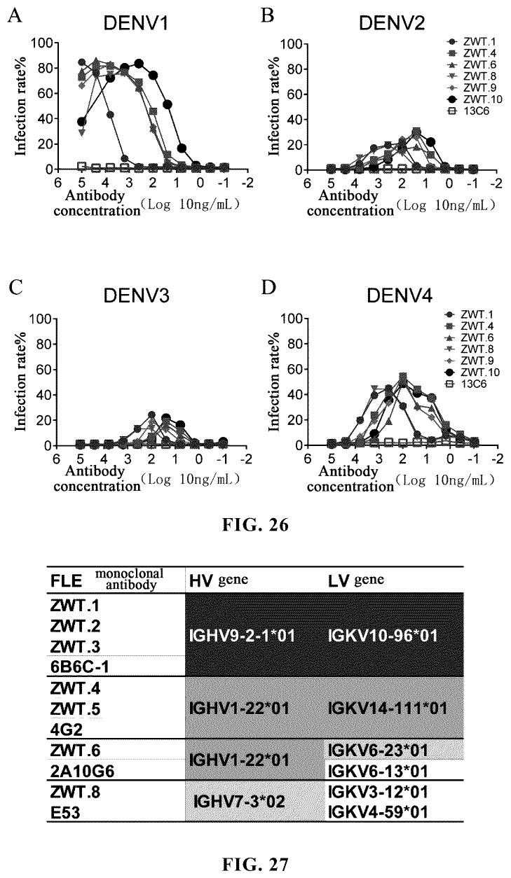

We expressed and purified representative FLE monoclonal antibodies derived from the ME-WT group to further evaluate the ADE effect of these antibodies on DENV on K562 cells. The results were shown in FIG. 26. It can be seen from FIG. 26 that all the tested antibodies derived from the ME-WT group had a certain degree of enhanced infection effect on the four serotypes of DENV. Among them, in FIG. 26, ZWT.1, 4, 6, 8, 9, and 10 were monoclonal antibodies from the M/E-WT group. DENV 1, DENV 2, DENV 3, and DENV 4 were serotypes 1-4 of the DENV virus, respectively. The results of the enhanced infection of K562 cells promoted by the four serotypes of DENV by ZWT.1, 4, 6, 8, 9, 10 antibodies were from A to D.

The FLE monoclonal antibodies derived from the ME-WT group were mainly composed of four types of HV:LV genes. We tried to search the literature and databases for the previously reported Flavivirus FLE murine monoclonal antibodies, which were then compared with the antibody loci and sequences we isolated. The following four monoclonal antibodies were found:
6B6C-1, which was isolated from tick-borne encephalitis virus (TBEV) after infection (Crill et al, (2004) Journal of Virology, 78.24: 13975-13986.);
both 4G2 and 2A10G6, which were isolated after DENV infection (Bennett et al. (2015), BMC Biotechnology, 15.1:71-71; Deng, Yongqiang, et al. (2011), PLOS ONE 6.1);
E53 which was isolated after WNV infection (Oliphant et al. (2006), Journal of Virology 80.24: 12149-12159.).

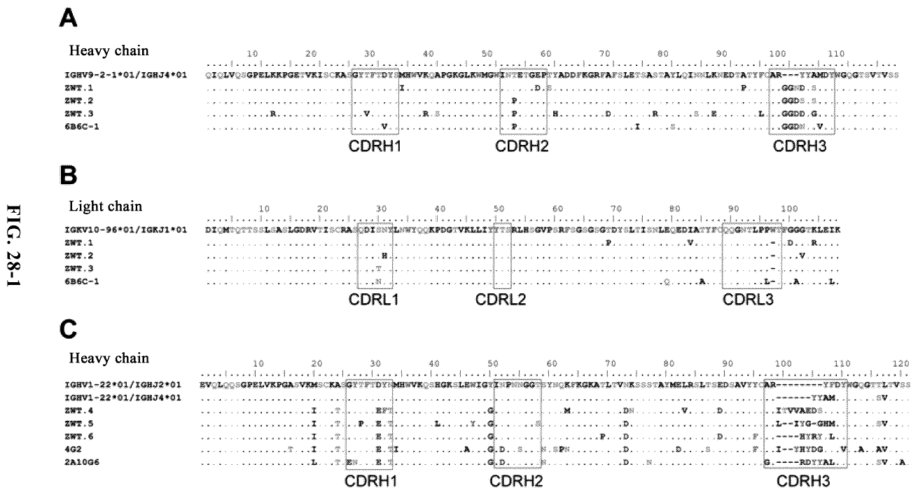

After analysis, it was found that the FLE monoclonal antibodies we isolated from the ME-WT group were more similar to the loci and sequences of these four reported monoclonal antibodies, as shown in FIG. 27, FIG. 28-1 and FIG. 28-2. It can be seen from FIG. 27 that 6B6C-1 and 4G2 use the same HV:LV gene pair as ZWT.1-3 and ZWT.4-5, respectively, and have a high sequence similarity; 2A10G6 and E53 use the same HV gene as ZWT.6 and ZWT.8, respectively.

Although the LV genes used by 2A10G6 and ZWT.6 were different, the LV sequences of the two antibodies were somewhat similar. As can be seen from FIG. 28-2, the CDRL3 and FR4 of 2A10G6 and ZWT.6 were identical.

From the above analysis, it was found that the FLE monoclonal antibody cloned from the lymph node GC B cells of mice immunized with the AdC7-M/E-WT vaccine had a locus that was close to or even the same as the reported mouse FLE monoclonal antibody, and the sequences were relatively similar. It showed that the locus used to induce antibodies that bound to the FL epitope in mice had a preference, and the characteristics of the produced FLE antibodies were also relatively similar.

In FIG. 28-1, A showed the sequence alignment analysis of the antibody heavy chains of ZWT.1, ZWT.2, ZWT.3, and 6B6C-1 and the mouse locus, B showed the sequence alignment of the antibody light chain of ZWT.1, ZWT.2, ZWT.3, and 6B6C-1 and the mouse locus, and C showed the sequence alignment of the antibody heavy chain of ZWT.4, ZWT.5, ZWT.6, 4G2, and 2A10G6 and the mouse locus. In FIG. 28-2, D showed the sequence alignment of antibody light chain of ZWT.4, ZWT.5, and 4G2 and the mouse locus, and E showed the sequence alignment of antibody light chain of ZWT.6, and 2A10G6 and the mouse locus.

### Example 17 SPR Assay to Detect the Affinity of Wild-Type and Mutant ZIKV E Proteins to ZIKV Antibodies

Since the AdC7-M/E-MutB and AdC7-M/E-MutC vaccines were able to provide complete protection in mice while avoiding ADE against DENV, we expressed purified soluble sE-MutC protein as a representative to compare with sE-WT to explain the underlying molecular mechanism.

BIOCORE8000 was based on the principle of Surface Plasmon Resonance (SPR), which can detect the interaction between molecules, reflect the dynamic changes in the process of molecular binding in real time, and obtain the kinetic parameters of the interaction.

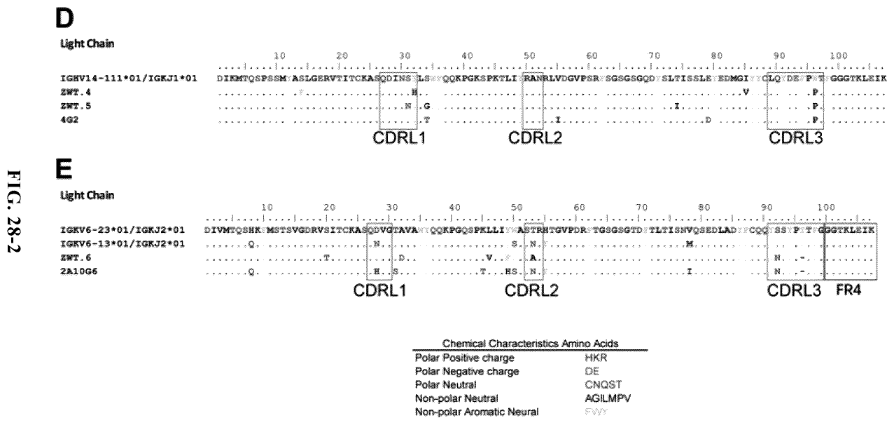

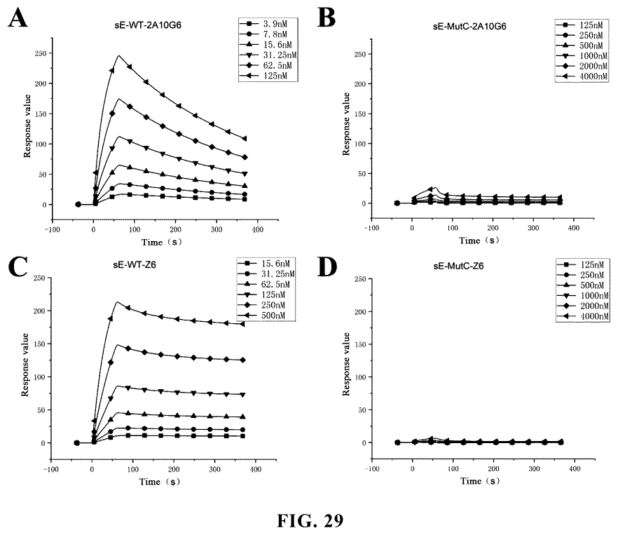

The affinity of ZIKV sE-WT protein and ZIKV sE-MutC protein to FLE antibody and non-FLE neutralizing antibody was detected using BIOCORE8000. Using a method of amino coupling, ZIKV sE-WT protein and ZIKV sE-MutC protein were immobilized on CM5 chip, respectively, and then four kinds of antibodies were diluted as mobile phase, which passes through fixed ZIKV sE protein. The corresponding signals were obtained when binding differently. The data was collected, fitted and computed. The results were shown in FIG. 29 and FIG. 30.

In FIG. 29, from A to D were the binding results of ZIKV sE-WT protein and 2A10G6 antibody (SE-WT-2A10G6), the binding results of ZIKV sE-MutC protein and 2A10G6 antibody (sE-MutC-2A10G6), binding results of ZIKV sE-WT protein and Z6 antibody (sE-WT-Z6), and binding results of ZIKV sE-MutC protein and Z6 antibody (sE-MutC-Z6), respectively. Among them, it can be seen from panel A in FIG. 29 that the affinity of 2A10G6 antibody binding to ZIKV sE-WT protein was 9.13 nM. From panel C in FIG. 29, it can be seen that the affinity of Z6 antibody binding to ZIKV sE-WT protein was 7.14 nM. From panel B in FIG. 29, it can be seen that ZIKV sE-MutC protein did not bind to 2A10G6 antibody at all. From panel D in FIG. 29, it can be seen that ZIKV sE-MutC protein did not bind to Z6 antibody at all. This result was also consistent with the theoretical analysis.

In FIG. 30, from A to D were the binding results of ZIKV sE-WT protein and Z3L1 antibody (SE-WT-Z3L1), the binding results of ZIKV sE-MutC protein and Z3L1 antibody (sE-MutC-Z3L1), binding results of ZIKV sE-WT protein and Z23 antibody (sE-WT-Z23), and binding results of ZIKV sE-MutC protein and Z23 antibody (sE-MutC-Z23), respectively. Among them, from panel A and panel C in FIG. 30, it can be seen that Z3L1 and Z23 antibodies bind to ZIKV E-WT protein with affinities of 9.48 μM and 0.625 μM, respectively. From panel B and panel D in FIG. 30, it can be seen that Z3L1 and Z23 antibodies bind to ZIKV sE-MutC protein with affinities of 8.01 μM and 0.701 μM, respectively. The affinity of the mutant protein ZIKV sE-MutC to Z23 and Z3L1 was almost unchanged compared with that of the wild-type ZIKV sE-WT protein. This indicated that the designed mutant could maintain the overall conformation of the protein and no change was made to epitopes other than the mutated site.

### Example 18 Structure Analysis of Complex of Mutant ZIKV sE-MutC Protein and Z3L1 Antibody

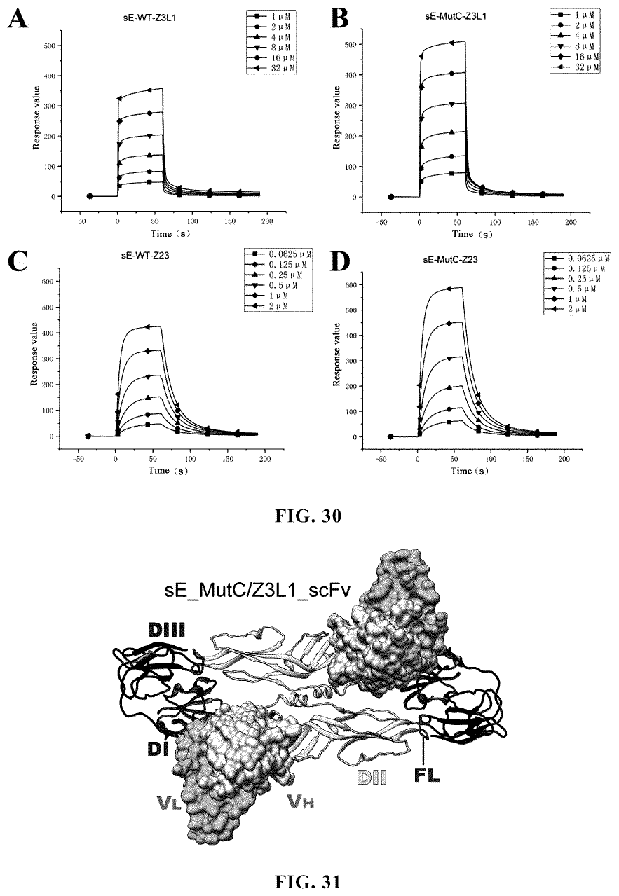

To further explain the mechanism of action of the mutant vaccine, we purified the complex protein of ZIKV sE-MutC and Z3L1 single-chain variable fragment (scFv), followed by crystal screening. Through X-ray diffraction analysis and structure analysis, the atomic structure of the complex with a resolution of 3 Å was obtained, as shown in FIG. 31. The data collection and optimized parameters of the complex were shown in Table 7.

TABLE 7
Data collection and optimized parameters
for ZIKV sE MutC-Z3L1 complexes

| | ZIKV sE-Z3L1 | |
|---|---|---|
| Data collection | | |
| Space group | P 21 21 21 | |
| Wavelength (Å) | 1.03923 | |
| Cell parameter | | |
| a, b, c(Å) | 78.46, 103.79, 205.82 | |
| α, β, γ(°) | 90.00, 90.00, 90.00 | |
| Resolution (Å) | 50.00-3.10 | (3.21-3.10) |
| Observed reflection | 288956 | |
| Integrity (%) | 99.2 | (99.9) |
| Redundancy | 9.3 | (9.8) |
| Rpim (%) | 5.6 | (30.1) |
| I/σ | 12.4 | (2.2) |

| | |
|---|---|
| Refinement | |
| Rwork/Rfree(%) | 22.23/26.80 |
| No. atoms | |
| Protein | 9754 |
| Ligand | 0 |
| Water | 0 |
| B-factor | |
| Protein | 69.80 |
| Ligand | 0 |
| Water | 0 |
| r.m.s. deviation | |
| Bond length (Å) | 0.002 |
| Bond angle (°) | 0.491 |
| Ramachandran plot | |
| Favoured(%) | 100 |
| Allowed(%) | 0 |
| Outliers(%) | 0 |

Values in the parentheses indicate the highest resolution of the shell

As can be seen from FIG. 31, despite the introduction of 5 point mutations into the ZIKV E protein FL sequence, sE-MutC still bound to the scFV of Z3L1 in a dimer form as the state before fusion. A detailed analysis of the dimer contact interface showed that the mutated FL amino acid residues established a new interaction between two adjacent E protein domain, and generated four and one hydrogen bonds at N98 and W108, respectively, which were beneficial for the stabilization of E protein dimers, as shown in Table 8.

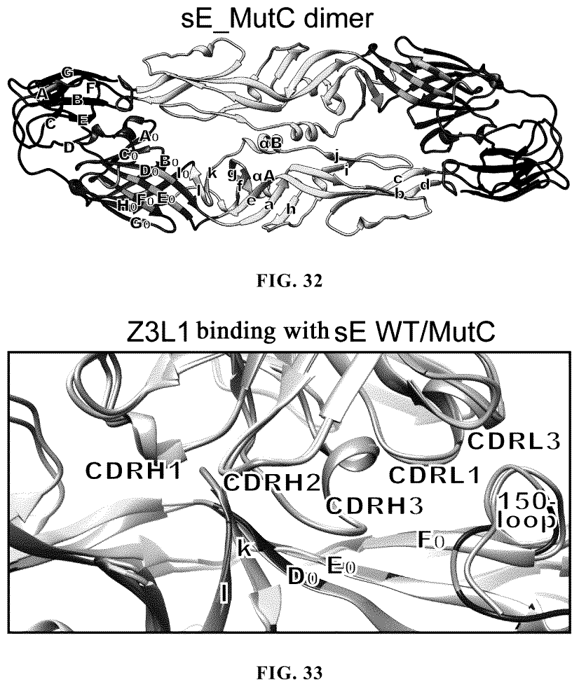

As can be seen from FIG. 32, the folded form of sE-MutC was very similar to that of the wild-type protein, showing normal secondary, tertiary and quaternary epitope structures.

To analyze the conformation of the neutralizing epitope, we overlapped the complex structures of ZIKV sE-MutC and Z3L1 scFv (Z3L1/ZIKV SE MutC) with the complex structures of Z3L1 and ZIKV sE-WT proteins (Z3L1/ZIKV SE WT, PDB: 5GZN). The results were shown in FIG. 33. It can be seen from FIG. 33 that the ZIKV wild-type protein sE-WT and mutant protein sE-MutC have the same binding mode to Z3L1 antibody, and the binding sites were mainly D0, E0 and F0 strands and 150 loop in DI, as well as kl hairpin in DII (Wang, et al. (2016) Science translational medicine 8.369:369ra179.).

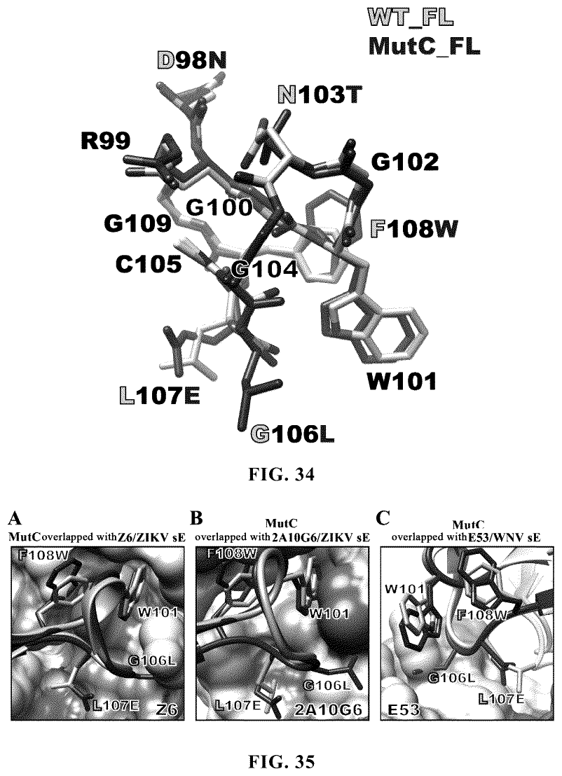

The FL region of sE-MutC and sE-WT were overlapped and analyzed for comparison. The results were shown in FIG. 34. It can be seen from FIG. 34 that the conformations of the FL epitopes of both were very similar, and only differs in the side chains of the mutation site.

To analyze the possibility of the mutant antigen sE-MutC inducing FLE antibodies, we overlapped the structures of FLE antibody having a known structure (Z6 antibody, 2A10G6 antibody and E53 antibody) that bound to Flavivirus E protein with the DII structure of sE-MutC for analysis. The results were shown in FIG. 35.

In FIG. 35, the complex structures of Z6 antibody and ZIKV sE protein, 2A10G6 antibody and ZIKV sE protein (PDB: 5JHL), and E53 antibody and WNV sE protein (PDB: 3I50) were shown in A to C, respectively. It can be seen from FIG. 35 that after the imitated monoclonal antibody binds to sE-MutC, the mutations of G106, L107 and F108 will obviously hinder the binding of antigen and antibody. The protruding long side chain of G106L will lead to conflict with the binding of these FLE antibodies. The L107E mutation allows the formation of charged side chains, which may disrupt the local hydrophobic interactions in the interaction. In addition, the F108W mutation creates a steric hindrance that affects the interaction between the FL epitope of sE-MutC and the 2A10G6 antibody. Therefore, on the FL epitope of ZIKV E protein, the three key amino acid mutations G106, L107 and F108 achieve a synergistic effect, eliminating the induction of FLE antibodies.

TABLE 8
Analysis of amino acid interactions of two protomers in ZIKV E dimers

| | | | | Total |
|---|---|---|---|---|
| Domain A | | Contact^{a} | Domain B | contacts |
| FL region | N98 | 5, 3, 7, 4 | G5 (1)^{b}, L322, S7 (3), V6 | 150 |
| | W101 | 3, 18, 5, 16, 5, 3, 5, 3 | I1, K316, M375, T327, A319, I317, | |
| | | | V328, E329 | |
| | G102 | 9 | I1 | |
| | L106 | 1 | A319 | |
| | E107 | 1 | A319 | |
| | W108 | 5, 9, 13, 1, 10, 11, 3, 3 | A319, E320, T321, L322, T327 (1), | |
| | | | I4, G5, I1 | |
| | G109 | 5 | L322 | |
| | K110 | 2 | S7 (1) | |
| Non-FL | I1 | 4, 7, 1 | W108, G102, W101 | 267 |
| region | I4 | 11 | W108 | |
| | G5 | 5, 4 | W108, N98 (1) | |
| | V6 | 2 | N98 | |
| | S7 | 7, 3 | N98 (1), K110 (1) | |
| | V153 | 1, 3 | T103, G102 | |
| | K209 | 3, 4 | K246, V256 | |
| | E244 | 2 | K209 | |
| | K246 | 8, 5 | E274, K209 | |
| | V256 | 5 | K209 | |
| | L258 | 2 | H266 | |
| | G259 | 8, 5, 6 | E262, H266, G263 | |
| | S260 | 3, 7, 1, 1 | E262, G263 (1), S260, A264 | |
| | Q261 | 10, 3, 1 | G263 (1), T267, A264 | |
| | E262 | 1, 4 | S260, G259 | |
| | G263 | 7, 4, 9 | G259, S260 (1), Q261 (1) | |
| | A264 | 1, 2, 5 | S260, Q261, A264 | |
| | H266 | 3, 4 | L258, G259 | |
| | T267 | 3 | Q261 | |
| | E274 | 9, 1 | K246 (2), E244 | |
| | K316 | 16, 2, 1 | W101, L106, C105 | |
| | I317 | 2 | W101 | |
| | A319 | 7, 3, 1, 1 | W101, W108, L106, E107 | |
| | E320 | 7 | W108 | |
| | T321 | 9 | W108 | |
| | L322 | 1, 3 | W108, N98 | |
| | T327 | 16, 8 | W101, W108 (1) | |
| | V328 | 6 | W101 | |
| | E329 | 3 | W101 | |
| | M375 | 6 | W101 | |

^{a}Numbers represent the number of atom-to-atom contacts between amino acid residues of the two domains, analysed using the CCP4 suite of programs (the threshold for distance is 4.5 Å).
^{b}The numbers in parentheses represent the number of possible hydrogen bonds between two amino acid residues.

### Example 19 Construction of DENV Vaccines With MutA, MutB and MutC Mutants

How to avoid ADE in the design of DENV vaccine was still an open question (REF). The FL sequence of ZIKV was very conservative with that of DENV. The experiments of the above system proved that AdC7-M/E-MutB and AdC7-M/E-MutC adenovirus vaccine could avoid ADE to DENV, and at the same time provided complete protection to mice. Therefore, DENV vaccines with MutA, MutB and MutC mutations were constructed to verify whether the vaccines have similar effects.

First, the signal peptide gene (SEQ ID NO. 17) derived from JEV (Japanese encephalitis virus) and the M/E gene (SEQ ID NO. 18) expressing DENV2 of New Guinea C strain (GenBank: KM204118.1) were constructed into pshuttle vector to obtain plasmid pshuttle-DV2-M/E-WT expressing wild-type DENV2 M/E. The signal peptide gene (SEQ ID NO. 17) derived from JEV and the gene expressing the prM/E protein of DENV2 New Guinea C strain (SEQ ID NO. 19) were constructed into pshuttle vector to obtain the wild-type DENV2 prM/E plasmid pshuttle-DV2-prM/E-WT.

All mutants were constructed based on M/E-WT antigen. MutA (D98N, N103T, G106F, L107E and F108W), MutB (D98N, N103T, G106F, L107K and F108W) and MutC (D98N, N103T, G106L, L107E and F108W) mutant plasmids were constructed using pshuttle-DV2-M/E-WT plasmid as template. The primer sequences used in the construction process were shown in Table 9.

TABLE 9
Primers used in the construction of pshuttle-DV2-M/E-WT, pshuttle-DV2-
prM/E-WT, and pshuttle-DV2-M/E-MutA/B/C

| Plasmid | Name of primer | Primer sequence (5′-3′) |
|---|---|---|
| pshuttle- | DV2-prM/E-F | AAACGGGCCCTCTAGAGCCACCATGCTCAAC (SEQ ID NO. 20) |
| DV2-pr | DV2-E-R | TTTAACTTAAGCTTGGTACCTTAAGCTTGCACCATGACTCCC |
| M/E-WT | | (SEQ ID NO. 21) |
| pshuttle- | DV2-M/E-F1 | AAACGGGCCCTCTAGAGCCACCATGCTCAACATTTTAAACAG |
| DV2-M/ | | AAGGAGGAGAACCGCCGGAATGATCATCATG (SEQ ID NO. 22) |
| E-WT | DV2-M/E-F2 | GGAGAACCGCCGGAATGATCATCATGCTGATCCCCACCGTGA |
| | | TGGCCAGCGTGGCTCTGGTGCCCCATGTC (SEQ ID NO. 23) |
| | DV2-E-R | TTTAACTTAAGCTTGGTACCTTAAGCTTGCACCATGACTCCC |
| | | (SEQ ID NO. 24) |
| pshuttle- | DV2-mutA-R | GTGAACAGAGGCTGGGGCACAGGATGCTTCGAATGGGGAAA |
| DV2-M/ | | GGGAGGCATCGTGACTTG (SEQ ID NO. 25) |
| E-MutA | DV2-mutA-F | TCCCCATTCGAAGCATCCTGTGCCCCAGCCTCTGTTCACCATG |
| | | GAGTGCTTGCACACGA (SEQ ID NO. 26) |
| pshuttle- | DV2-mutB-R | GTGAACAGAGGCTGGGGCACAGGATGCTTCAAGTGGGGAAA |
| DV2-M/ | | GGGAGGCATCGTGACTTG (SEQ ID NO. 27) |
| E-MutB | DV2-mutB-F | TCCCCACTTGAAGCATCCTGTGCCCCAGCCTCTGTTCACCATG |
| | | GAGTGCTTGCACACGA (SEQ ID NO. 28) |
| pshuttle- | DV2-mutC-R | GTGAACAGAGGATGGGGCACAGGATGCCTGGAATGGGGCAA |
| DV2-M/ | | GGGCTCTTTAATCACTTG (SEQ ID NO. 29) |
| E-MutC | DV2-mutC-F | GCCCCATTCCAGGCATCCTGTGCCCCATCCTCTGTTCACGAAG |
| | | GTCCTACGACACACGA (SEQ ID NO. 30) |

293T cells were transfected with plasmids pshuttle-DV2-M/E-WT and pshuttle-DV2-prM/E-WT expressing wild-type protein, and three mutant plasmids, respectively. After 48 hours, the supernatant was removed. The cells were washed once with PBS, then trypsinized into single cells, centrifuged, resuspended in DMEM medium, and washed again with DMEM medium. Then, the Fixation and Permeabilization solution from BD Company was added, and placing on ice for 20 minutes. Then, the cells were collected by centrifugation at 800 g for 10 minutes and washed twice with 1×Perm/Wash buffer from BD company. Each sample was divided into 5 parts, adding Z6, 2A10G6 and mAb11 antibodies that bound to FL epitopes and mAb513 and D448 antibodies that bound to non-FL epitopes, respectively, and placing in a refrigerator at 4° C. for 1 hour. The cells were harvested by centrifugation and washed twice with 1×Perm/Wash buffer. Next, Goat Anti-Human FITC (Proteintech, 00003-12) antibody was added, and placing in a refrigerator at 4° C. for 1 hour. The cells were harvested by centrifugation and washed twice with 1×Perm/Wash buffer. The cells were resuspended in PBS (200 μl per well).

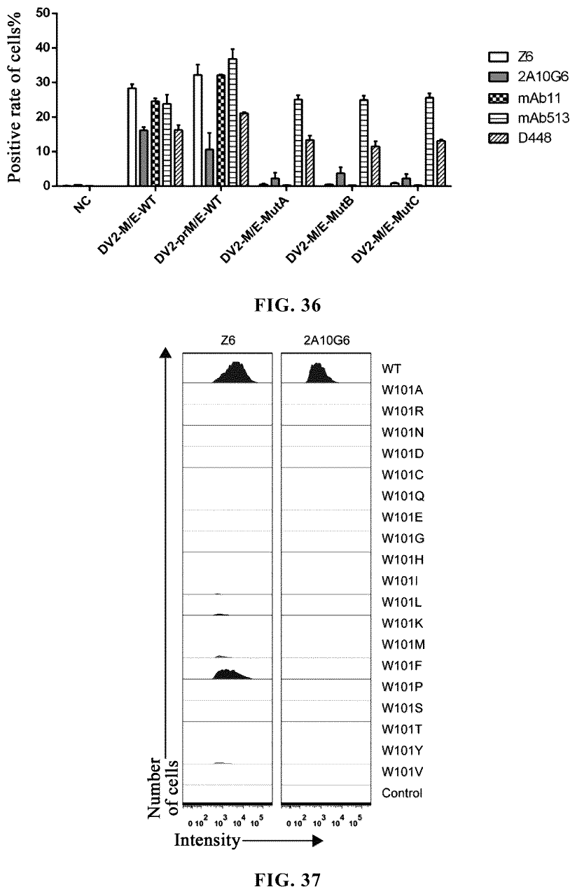

The positive proportion of the samples was detected by flow cytometry. The experimental results were shown in FIG. 36.

It can be seen from FIG. 36 that the antibodies Z6 and 2A10G6 that bound to the FL epitope could recognize the M/E-WT and prM/E-WT proteins of DENV2. However, after the FL epitope of the E protein of DENV2 was introduced with three mutation combinations, the binding ability of Z6 and 2A10G6 antibodies to the M/E-MutA or M/E-MutB or M/E-MutC proteins of DENV2 was greatly reduced or they even did not bind. For mAb513 and D448 antibodies that bound to non-FL epitopes with higher neutralizing activity, they could still bind to the M/E-MutA or M/E-MutB or M/E-MutC proteins of DENV2 introduced with MutA, MutB and MutC mutations, indicating that the mutation of the FL epitope of DENV2 E protein had no significant effect on other epitopes. The above results showed that these three mutation combinations could be used in DENV vaccines, and the vaccine obtained based on the DENV2 E protein antigen with the combination of G106, L107 and F108 site mutations may have reduced ADE effect caused by subsequent DENV virus infection after vaccine immunization.

### Example 20 Detection of the Effect of ZIKV E W101 Mutation to Other 19 Amino Acids on Antigenic Activity

In the structures of Z6/ZIKV sE and 2A10G6/ZIKV sE, W101 was the amino acid where E protein interacts most with antibodies. It had also been reported in the literature that most FLE antibodies bound to E protein by W101 (Dejnirattisai, W. et al (2015). Nat Immunol 16, 170-177.). Therefore, we tried to mutate W101 into other 19 amino acids, and then detected whether the epitope of the antigen expressed by cells was changed by cytometry to screen out the most suitable mutation.

The signal peptide gene (SEQ ID NO. 17) derived from JEV and the M/E gene (SEQ ID NO. 31) of wild-type ZIKV were constructed into pCAGGS vector (Addgene) to obtain the plasmid pCAGGS-M/E-WT that could express M/E protein of wild-type ZIKV. Using this plasmid as a template, tryptophan at position 101 of E protein was mutated into other 19 amino acids. Taking the mutation of tryptophan to alanine as an example, using plasmid pCAGGS-M/E-WT as a template, and using W101-WT-F and W101-1A-R as primers, PCR product W101-1A-1 was obtained. For the primer sequence, see Table 10. Using plasmid pCAGGS-M/E-WT as template and W101-WT-R and W101-1A-F as primers, PCR product W101-1A-2 was obtained. Then, W101-1A-1 and W101-1A-2 were mixed in a molar ratio of 1:1 as a template, and W101-WT-F and W101-WT-R were used as primers for PCR to obtain PCR product W101-1A. The pCAGGS vector was restricted with XhoI (Thermo, FD0694) and EcoRI (Thermo, FD0274) to obtain a linear plasmid with double cohesive ends. The digested linear plasmid was mixed with W101-1A according to a molar ratio of 1:5, and the In-Fusion kit (Takara, 639648) was used for recombination. The recombinant product was transformed into DH5α competent cells, spread on ampicillin-resistant plates, and cultured at 37° C. After that, the clones were picked for PCR identification and sequencing identification.

TABLE 10
Primer for tryptophan mutation at position 101 of E protein of ZIKV

| Mutant | Name of | |
|---|---|---|
| form | primer | Primer sequence (5′-3′) |
| WT | W101-WT-F | TTTTGGCAAAGAATTCGCCG (SEQ ID NO. 32) |
| | W101-WT-R | GATCTGCTAGCTCGAGTCAAGCGCTCACAGCTGTGGACAGA |
| | | (SEQ ID NO. 33) |
| W101A | W101-1A-R | CGCAGCCATTTCCGGCGCCCCTGTCCACCAGGGTCC (SEQ ID |
| | | NO. 34) |
| | W101-1A-F | GGTGGACAGGGGCGCCGGAAATGGCTGCGGCCTGTTTG (SEQ |
| | | ID NO. 35) |
| W101R | W101-2R-R | CGCAGCCATTTCCCCGGCCCCTGTCCACCAGGGTCC (SEQ ID |
| | | NO. 36) |
| | W101-2R-F | GGTGGACAGGGGCCGGGGAAATGGCTGCGGCCTGTTTG (SEQ |
| | | ID NO. 37) |
| W101N | W101-3N-R | CGCAGCCATTTCCGTTOCCCCTGTCCACCAGGGTCC (SEQ ID |
| | | NO. 38) |
| | W101-3N-F | GGTGGACAGGGGCAACGGAAATGGCTGCGGCCTGTTTG (SEQ |
| | | ID NO. 39) |
| W101D | W101-4D-R | CGCAGCCATTTCCGTCGCCCCTGTCCACCAGGGTCC (SEQ ID |
| | | NO. 40) |
| | W101-4D-F | GGTGGACAGGGGCGACGGAAATGGCTGCGGCCTGTTTG (SEQ |
| | | ID NO. 41) |
| W101C | W101-5C-R | CGCAGCCATTTCCGCAGCCCCTGTCCACCAGGGTCC (SEQ ID |
| | | NO. 42) |
| | W101-5C-F | GGTGGACAGGGGCTGCGGAAATGGCTGCGGCCTGTTTG (SEQ |
| | | ID NO. 43) |
| W101Q | W101-6Q-R | CGCAGCCATTTCCCTGGCCCCTGTCCACCAGGGTCC (SEQ ID |
| | | NO. 44) |
| | W101-6Q-F | GGTGGACAGGGGCCAGGGAAATGGCTGCGGCCTGTTTG (SEQ |
| | | ID NO. 45) |
| W101E | W101-7E-R | CGCAGCCATTTCCCTCGCCCCTGTCCACCAGGGTCC (SEQ ID |
| | | NO. 46) |
| | W101-7E-F | GGTGGACAGGGGCGAGGGAAATGGCTGCGGCCTGTTTG (SEQ |
| | | ID NO. 47) |
| W101G | W101-8G-R | CGCAGCCATTTCCGCCGCCCCTGTCCACCAGGGTCC (SEQ ID |
| | | NO. 48) |
| | W101-8G-F | GGTGGACAGGGGCGGCGGAAATGGCTGCGGCCTGTTTG (SEQ |
| | | ID NO. 49) |
| W101H | W101-9H-R | CGCAGCCATTTCCGTGGCCCCTGTCCACCAGGGTCC (SEQ ID |
| | | NO. 50) |
| | W101-9H-F | GGTGGACAGGGGCCACGGAAATGGCTGCGGCCTGTTTG (SEQ |
| | | ID NO. 51) |
| W101I | W101-10I-R | CGCAGCCATTTCCGATGCCCCTGTCCACCAGGGTCC (SEQ ID |
| | | NO. 52) |
| | W101-10I-F | GGTGGACAGGGGCATCGGAAATGGCTGCGGCCTGTTTG (SEQ |
| | | ID NO. 53) |
| W101L | W101-11L-R | CGCAGCCATTTCCCAGGCCCCTGTCCACCAGGGTCC (SEQ ID |
| | | NO. 54) |
| | W101-11L-F | GGTGGACAGGGGCCTGGGAAATGGCTGCGGCCTGTTTG (SEQ |
| | | ID NO. 55) |
| W101K | W101-12K-R | CGCAGCCATTTCCCTTGCCCCTGTCCACCAGGGTCC (SEQ ID |
| | | NO. 56) |
| | W101-12K-F | GGTGGACAGGGGCAAGGGAAATGGCTGCGGCCTGTTTG (SEQ |
| | | ID NO. 57) |
| W101M | W101-13M-R | CGCAGCCATTTOCCATGCCCCTGTCCACCAGGGTCC (SEQ ID |
| | | NO. 58) |
| | W101-13M-F | GGTGGACAGGGGCATGGGAAATGGCTGCGGCCTGTTTG (SEQ |
| | | ID NO. 59) |
| W101F | W101-14F-R | CGCAGCCATTTCCGAAGCCCCTGTCCACCAGGGTCC (SEQ ID |
| | | NO. 60) |
| | W101-14F-F | GGTGGACAGGGGCTTCGGAAATGGCTGCGGCCTGTTTG (SEQ |
| | | ID NO. 61) |
| W101P | W101-15P-R | CGCAGCCATTTCCGGGGCCCCTGTCCACCAGGGTCC (SEQ ID |
| | | NO. 62) |
| | W101-15P-F | GGTGGACAGGGGCCCCGGAAATGGCTGCGGCCTGTTTG (SEQ |
| | | ID NO. 63) |
| W101S | W101-16S-R | CGCAGCCATTTCCGCTGCCCCTGTCCACCAGGGTCC (SEQ ID |
| | | NO. 64) |
| | W101-16S-F | GGTGGACAGGGGCAGCGGAAATGGCTGCGGCCTGTTTG (SEQ |
| | | ID NO. 65) |
| W10IT | W101-17T-R | CGCAGCCATTTCCGGTGCCCCTGTCCACCAGGGTCC (SEQ ID |
| | | NO. 66) |
| | W101-17T-F | GGTGGACAGGGGCACCGGAAATGGCTGCGGCCTGTTTG (SEQ |
| | | ID NO. 67) |
| W101Y | W101-19Y-R | CGCAGCCATTTCCGTAGCCCCTGTCCACCAGGGTCC (SEQ ID |
| | | NO. 68) |
| | W101-19Y-F | GGTGGACAGGGGCTACGGAAATGGCTGCGGCCTGTTTG (SEQ |
| | | ID NO. 69) |
| W101V | W101-20V-R | CGCAGCCATTTOCCACGCCCCTGTCCACCAGGGTCC (SEQ ID |
| | | NO. 70) |
| | W101-20V-F | GGTGGACAGGGGCGTGGGAAATGGCTGCGGCCTGTTTG (SEQ |
| | | ID NO. 71) |

After extraction of 19 mutant plasmids of W101, 293T cells were transfected with wild-type plasmids and 19 mutant plasmids, respectively. After 48 hours, the supernatant was removed. The cells were washed once with PBS, then trypsinized into single cells, centrifuged, resuspended in DMEM medium, and washed again with DMEM medium. Then, the Fixation and Permeabilization solution from BD Company was added, and placing on ice for 20 minutes. Then, the cells were collected by centrifugation at 800 g for 10 minutes and washed twice with 1×Perm/Wash buffer from BD company. Each sample was divided into 5 parts, adding Z6 and 2A10G6 antibodies respectively, and placing in a refrigerator at 4° C. for 1 hour. The cells were harvested by centrifugation and washed twice with 1×Perm/Wash buffer. Next, Goat Anti-Human FITC (Proteintech, 00003-12) antibody was added, and placing in a refrigerator at 4° C. for 1 hour. The cells were harvested by centrifugation and washed twice with 1×Perm/Wash buffer. The cells were resuspended in PBS (200 μl per well). The positive proportion of the samples was detected by flow cytometry. The experimental results were shown in FIG. 37.

It can be seen from FIG. 37 that ZIKV wild-type M/E antigen could bind to Z6 and 2A10G6 antibodies, while all 19 mutants can hardly bind to Z6 and 2A10G6 antibodies, indicating that tryptophan at position 101 of ZIKV E protein was important for activating antibodies targeting FL. The vaccine prepared by the W101 site mutation will reduce or avoid the production of FL epitope-induced antibodies, thereby avoiding the ADE effect on DENV after vaccine immunization.

### Example 21 Detecting the Binding Ability of ZIKV E Protein With G106, L107 and F108 Site Mutations on FLE Antibody

Based on the above-mentioned pCAGGS-ZIKV-M/E expression plasmid, the following single-site and double-site mutations were performed. The construction method of the mutant plasmid referred to Example 20. For the primers used in the construction of the mutant plasmid, see Table 11.

TABLE 11
Primers used for sites mutation of G106, L107 and F108 of ZIKV E protein

| Mutation site | Mutation form | Upstream and downstream primers |
|---|---|---|
| G106 | G106L | G106L-F: CTGCT GTTTG GAAAG GGCTC CCTGG TGACC |
| mutation | | TG (SEQ ID NO. 72) |
| | | G106L-R: AAACAGCAGGCAGCCATTT CCCCAGC CCC |
| | | TGT (SEQ ID NO. 73) |
| | G106F | G106F-F: TTCCTGTTTGGAAAGGGCTCCCTGGTGAC CT |
| | | G (SEQ ID NO. 74) |
| | | G106F-R: AAACAGGAAGCAGCCATTTCCCCAGCCCC |
| | | TGT (SEQ ID NO. 75) |
| | G106W | G106W-F: TGGCTGTTTGGAAAGGGCTCCCTGGTGACC |
| | | TG (SEQ ID NO. 76) |
| | | G106W-R: AAACAGCCAGCAGCCATTTCCCCAGCCCCT |
| | | GT (SEQ ID NO. 77) |
| | G106Y | G106Y-F: TACCTGTTTGGAAAGGGCTCCCTGGTGACC |
| | | TG (SEQ ID NO. 78) |
| | | G106Y-R: AAACAGGTAGCAGCCATTTCCCCAGCCCCT |
| | | GT (SEQ ID NO. 79) |
| | G106I | G106I-F: ATCCTGTTTGGAAAGGGCTCCCTGGTGACCTG |
| | | (SEQ ID NO. 80) |
| | | G106I-R: AAACAGGATGCAGCCATTTCCCCAGCCCCTG T |
| | | (SEQ ID NO. 81) |
| L107 | L107E | L107E-F: GGCGAGTTTGGAAAGGGCTCCCTGGTGACC |
| mutation | | TG (SEQ ID NO. 82) |
| | | L107E-R: AAACTCGCCGCAGCCATTTCCCCAGCCCCT GT |
| | | (SEQ ID NO. 83) |
| | L107K | L107K-F: GGCAAGTTTGGAAAGGGCTCCCTGGTGACCT |
| | | G (SEQ ID NO. 84) |
| | | L107K-R: AAACTTGCCGCAGCCATTTCCCCAGCCCCTG T |
| | | (SEQ ID NO. 85) |
| | L107R | L107R-F: GGCCGGTTTGGAAAGGGCTCCCTGGTGACCT |
| | | G (SEQ ID NO. 86) |
| | | L107R-R: AAACCGGCCGCAGCCATTTCCCCAGCCCCTG |
| | | T (SEQ ID NO. 87) |
| | L107D | L107D-F: GGCGACTTTGGAAAGGGCTCCCTGGTGACC |
| | | TG (SEQ ID NO. 88) |
| | | LI07D-R: AAAGTCGCCGCAGCCATTTCCCCAGCCCCT |
| | | GT (SEQ ID NO. 89) |
| | L107T | L107T-F: GGCACCTTTGGAAAGGGCTCCCTGGTGACC |
| | | TG (SEQ ID NO. 90) |
| | | L107T-R: AAAGGTGCCGCAGCCATTTCCCCAGCCCCT GT |
| | | (SEQ ID NO. 91) |
| F108 | F108W | F108W-F: GGCCTGTGGGGAAAGGGCTCCCTGGTGA |
| mutation | | CCTG (SEQ ID NO. 92) |
| | | F108W-R: CCACAGGCCGCAGCCATTTCCCCAGCCCCT |
| | | GT (SEQ ID NO. 93) |
| | F108H | F108H-F: GGCCTGCACGGAAAGGGCTCCCTGGTGACC |
| | | TG (SEQ ID NO. 94) |
| | | F108H-R: GTGCAGGCCGCAGCCATTTCCCCAGCCCCTG |
| | | T (SEQ ID NO. 95) |
| | F108Y | F108Y-F: GGCCTGTACGGAAAGGGCTCCCTGGTGACCT |
| | | G (SEQ ID NO. 96) |
| | | F108Y-R: GTACAGGCCGCAGCCATTTCCCCAGCCCCTG T |
| | | (SEQ ID NO. 97) |
| | F108P | F108P-F: GGCCTGCCCGGAAAGGGCTCCCTGGTGACCT |
| | | G (SEQ ID NO. 98) |
| | | F108P-R: GGGCAGGCCGCAGCCATTTCCCCAGCCCCTG T |
| | | (SEQ ID NO. 99) |
| | F108A | F108A-F: GGCCTGGCCGGAAAGGGCTCCCTGGTGACCT |
| | | G (SEQ ID NO. 100) |
| | | F108A-R: GGCCAGGCCGCAGCCATTTCCCCAGCCCCTG |
| | | T (SEQ ID NO. 101) |
| G106/L107 | G106L/L107E | G106L/L107E-F: CTGGAGTTTGGAAAGGGCTCCCTGGT |
| mutation | | GACCTG (SEQ ID NO. 102) |
| | | G106L/L107E-R: AAACTCCAGGCAGCCATTTCCCCAGC |
| | | CCCTGT (SEQ ID NO. 103) |
| | G106L/L107K | G106L/L107K-F: CTGAAGTTTGGAAAGGGCTCCCTGG |
| | | TGACCTG (SEQ ID NO. 104) |
| | | G106L/L107K-R: AAACTTCAGGCAGCCATTTCCCCAG |
| | | CCCCTGT (SEQ ID NO. 105) |
| | G106F/L107E | G106F/L107E-F: TTCGAGTTTGGAAAGGGCTCCCTGGTG |
| | | ACCTG (SEQ ID NO. 106) |
| | | G106F/L107E-R: AAACTCGAAGCAGCCATTTCCCCAGC |
| | | CCCTGT (SEQ ID NO. 107) |
| | G106F/L107K | G106F/L107K-F: TTCAAGTTTGGAAAGGGCTCCCTGGT |
| | | GACCTG (SEQ ID NO. 108) |
| | | G106F/L107K-R: AAACTTGAAGCAGCCATTTCCCCAGC |
| | | CCCTGT (SEQ ID NO. 109) |
| | G106F/L107R | G106F/L107R-F: TTCCGGTTTGGAAAGGGCTCCCTGGTG |
| | | ACCTG (SEQ ID NO. 110) |
| | | G106F/L107R-R: AAACCGGAAGCAGCCATTTCCCCAGC |
| | | CCCTGT (SEQ ID NO. 111) |
| G106/F108 | G106F/F108W | G106F/F108W-F: TTCCTGTGGGGAAAGGGCTCCCTGGT |
| mutation | | GACCTG (SEQ ID NO. 112) |
| | | G106F/F108W-R: CCACAGGAAGCAGCCATTTCCCCAG |
| | | CCCCTGT (SEQ ID NO. 113) |
| | G106L/F108W | G106L/F108W-F: CTGCTGTGGGGAAAGGGCTCCCTGGT |
| | | GACCTG (SEQ ID NO. 114) |
| | | G106L/F108W-R: CCACAGCAGGCAGCCATTTCCCCAGC |
| | | CCCTGT (SEQ ID NO. 115) |
| | G106F/F108H | G106F/F108H-F: TTCCTGCACGGAAAGGGCTCCCTGG |
| | | TGACCTG (SEQ ID NO. 116) |
| | | G106F/F108H-R: GTGCAGGAAGCAGCCATTTCCCCAG |
| | | CCCCTGT (SEQ ID NO. 117) |
| | G106Y/F108W | G106Y/F108W-F: TACCTGTGGGGAAAGGGCTCCCTGG |
| | | TGACCTG (SEQ ID NO. 118) |
| | | G106Y/F108W-R: CCACAGGTAGCAGCCATTTCCCCAGC |
| | | CCCTGT (SEQ ID NO. 119) |
| | G106W/F108Y | G106W-F: TGGCTGTACGGAAAGGGCTCCCTGG |
| | | TGACCTG (SEQ ID NO. 120) |
| | | G106W-R: GTACAGCCAGCAGCCATTTCCCCAGCCC |
| | | CTGT (SEQ ID NO. 121) |
| L107/F108 | LI07E/F108W | L107E/F108W-F: GGCGAGTGGGGAAAGGGCTCCCTG |
| mutation | | GTGACCTG (SEQ ID NO. 122) |
| | | L107E/F108W-R: CCACTCGCCGCAGCCATTTCCCCAG |
| | | CCCCTGT (SEQ ID NO. 123) |
| | LI07K/F108W | L107K/F108W-F: GGCAAGTGGGGAAAGGGCTCCCTGGT |
| | | GACCTG (SEQ ID NO. 124) |
| | | L107K/F108W-R: CCACTTGCCGCAGCCATTTCCCCAG |
| | | CCCCTGT (SEQ ID NO. 125) |
| | L107R/F108W | L107R/F108W-F: GGCCGGTGGGGAAAGGGCTCCCTG |
| | | GTGACCTG (SEQ ID NO. 126) |
| | | L107R/F108W-R: CCACCGGCCGCAGCCATTTCCCCAG |
| | | CCCCTGT (SEQ ID NO. 127) |
| | L107D/F108W | L107D/F108W-F: GGCGACTGGGGAAAGGGCTCCCTG |
| | | GTGACCTG (SEQ ID NO. 128) |
| | | L107D/F108W-R: CCAGTCGCCGCAGCCATTTCCCCA |
| | | GCCCCTGT (SEQ ID NO. 129) |
| | L107K/F108Y | L107K/F108Y-F: GGCAAGTACGGAAAGGGCTCCCTG |
| | | GTGACCTG (SEQ ID NO. 130) |
| | | L107K/F108Y-R: GTACTTGCCGCAGCCATTTCCCCAGC |
| | | CCCTGT (SEQ ID NO. 131) |

The wild-type plasmid pCAGGS-ZIKV-M/E-WT and the successfully constructed mutant plasmid were transfected into 293T cells respectively. After 48 hours, the cells were harvested, digested into single cells, fixed and permeabilized, incubated with FL epitope-binding ADE antibodies Z6 and 2A10G6, and incubated with Goat Anti-Human (mouse) FITC secondary antibody. Finally, the positive proportion of samples was measured by flow cytometry. If the positive proportion was lower than 10% of the wild-type positive rate, it was considered as non-binding, and if 10%-50% of the wild-type positive rate, it was considered as weak binding. The results were shown in Table 12.

It can be seen from Table 12 that the above mutations could basically prevent the binding of the FL epitope representative ADE antibodies (Z6 antibody and 2A10G6 antibody), indicating that the vaccine prepared by the single point or synergistic mutation of G106, L107 and F108 sites represented by these mutations will reduce or avoid the production of the ADE antibodies that was induced by FL epitope, thereby avoiding the ADE effect on DENV after vaccine immunization.

TABLE 12
Results of ZIKV E protein mutants binding
to Z6 antibody and 2A10G6 antibody

FLE antibody

| Mutation site | Mutation form | Z6 | 2A10G6 |
|---|---|---|---|
| G106 mutation | G106L | — | — |
| | G106F | — | — |
| | G106W | — | — |
| | G106Y | — | — |
| | G106I | — | — |
| L107 mutation | L107E | — | — |
| | L107K | — | — |
| | L107R | — | — |
| | L107D | — | — |
| | L107T | — | — |
| F108 mutation | F108W | Weak binding | — |
| | F108H | — | — |
| | F108Y | — | — |
| | F108P | — | — |
| | F108A | — | — |
| G106/L107 | G106L/L107E | — | — |
| mutation | G106L/L107K | — | — |
| | G106F/L107E | — | — |
| | G106F/L107K | — | — |
| | G106F/L107R | — | — |
| G106/F108 | G106F/F108W | — | — |
| mutation | G106L/F108W | — | — |
| | G106F/F108H | — | — |
| | G106Y/F108W | — | — |
| | G106W/F108Y | — | — |
| L107/F108 | L107E/F108W | — | — |
| mutation | L107K/F108W | — | — |
| | L107R/F108W | — | — |
| | L107D/F108W | — | — |
| | L107K/F108Y | — | — |

Note:
— means no binding.

### Example 22 Detecting the Binding Ability of DENV E Protein With G106, L107 and F108 Site Mutations on FLE Antibody

Based on the above-mentioned pCAGGS-DENV2-M/E expression plasmid, the following single-site and double-site mutations were performed. The construction method of the mutant plasmid referred to Example 19. For the primers used in the construction of the mutant plasmid, see Table 13.

TABLE 13
Primers used for mutation of G106, L107 and F108 sites of DENV E protein

| Mutation site | Mutation form | Upstream and downstream primers |
|---|---|---|
| G106 | G106L | G106L-F: CTGTTATTCGGAAAGGGAGGCATCGTGACTTG |
| mutation | | (SEQ ID NO. 132) |
| | | G106L-R: GAATAACAGGCATCCATTGCCCCAGCCTCTGT |
| | | (SEQ ID NO. 133) |
| | G106F | G106F-F: TTCTTATTCGGAAAGGGAGGCATCGTGACTTG |
| | | (SEQ ID NO. 134) |
| | | G106F-R: GAATAAGAAGCATCCATTGCCCCAGCCTCTGT |
| | | (SEQ ID NO. 135) |
| | G106W | G106W-F: TGGTTATTCGGAAAGGGAGGCATCGTGACTTG |
| | | (SEQ ID NO. 136) |
| | | G106W-R: GAATAACCAGCATCCATTGCCCCAGCCTCTGT |
| | | (SEQ ID NO. 137) |
| | G106Y | G106Y-F: TACTTATTCGGAAAGGGAGGCATCGTGACTTG |
| | | (SEQ ID NO. 138) |
| | | G106Y-R: GAATAAGTAGCATCCATTGCCCCAGCCTCTGT(SEQ |
| | | ID NO. 139) |
| | G106I | G106I-F: ATCTTATTCGGAAAGGGAGGCATCGTGACTTG(SEQ |
| | | ID NO. 140) |
| | | G106I-R: GAATAAGATGCATCCATTGCCCCAGCCTCTGT(SEQ |
| | | ID NO. 141) |
| L107 | L107E | L107E-F: GGTGAGTTCGGAAAGGGAGGCATCGTGACTTG |
| mutation | | (SEQ ID NO. 142) |
| | | L107E-R: GAACTCACCGCATCCATTGCCCCAGCCTCTGT |
| | | (SEQ ID NO. 143) |
| | L107K | L107K-F: GGTAAGTTCGGAAAGGGAGGCATCGTGACTTG |
| | | (SEQ ID NO. 144) |
| | | L107K-R: GAACTTACCGCATCCATTGCCCCAGCCTCTGT(SEQ |
| | | ID NO. 145) |
| | L107R | L107R-F: GGTCGGTTCGGAAAGGGAGGCATCGTGACTTG |
| | | (SEQ ID NO. 146) |
| | | L107R-R: GAACCGACCGCATCCATTGCCCCAGCCTCTGT |
| | | (SEQ ID NO. 147) |
| | L107D | L107D-F: GGTGACTTCGGAAAGGGAGGCATCGTGACTTG |
| | | (SEQ ID NO. 148) |
| | | L107D-R: GAAGTCACCGCATCCATTGCCCCAGCCTCTGT |
| | | (SEQ ID NO. 149) |
| | L107T | L107T-F: GGTACCTTCGGAAAGGGAGGCATCGTGACTTG |
| | | (SEQ ID NO. 150) |
| | | L107T-R: GAAGGTACCGCATCCATTGCCCCAGCCTCTGT(SEQ |
| | | ID NO. 151) |
| F108 | F108W | F108W-F: GGTTTATGGGGAAAGGGAGGCATCGTGACTTG |
| mutation | | (SEQ ID NO. 152) |
| | | F108W-R: CCATAAACCGCATCCATTGCCCCAGCCTCTGT |
| | | (SEQ ID NO. 153) |
| | F108H | F108H-F: GGTTTACACGGAAAGGGAGGCATCGTGACTTG |
| | | (SEQ ID NO. 154) |
| | | F108H-R: GTGTAAACCGCATCCATTGCCCCAGCCTCTGT(SEQ |
| | | ID NO. 155) |
| | F108Y | F108Y-F: GGTTTATACGGAAAGGGAGGCATCGTGACTTG |
| | | (SEQ ID NO. 156) |
| | | F108Y-R: GTATAAACCGCATCCATTGCCCCAGCCTCTGT(SEQ |
| | | ID NO. 157) |
| | F108P | F108P-F: GGTTTACCCGGAAAGGGAGGCATCGTGACTTG |
| | | (SEQ ID NO. 158) |
| | | F108P-R: GGGTAAACCGCATCCATTGCCCCAGCCTCTGT(SEQ |
| | | ID NO. 159) |
| | F108A | F108A-F: GGTTTAGCCGGAAAGGGAGGCATCGTGACTTG |
| | | (SEQ ID NO. 160) |
| | | F108A-R: GGCTAAACCGCATCCATTGCCCCAGCCTCTGT |
| | | (SEQ ID NO. 161) |
| G106/L107 | G106L/L107E | G106L/L107E-F: CTGGAGTTCGGAAAGGGAGGCATCG |
| mutation | | TGACTTG (SEQ ID NO. 162) |
| | | G106L/L107E-R: GAACTCCAGGCATCCATTGCCCCAGCCTC |
| | | TGT (SEQ ID NO. 163) |
| | G106L/L107K | G106L/L107K-F: CTGAAGTTCGGAAAGGGAGGCATCGTG |
| | | ACTTG (SEQ ID NO. 164) |
| | | G106L/L107K-R: GAACTTCAGGCATCCATTGCCCCAGC |
| | | CTCTGT (SEQ ID NO. 165) |
| | G106F/L107E | G106F/L107E-F: TTCGAGTTCGGAAAGGGAGGCATC |
| | | GTGACTTG (SEQ ID NO. 166) |
| | | G106F/L107E-R: GAACTCGAAGCATCCATTGCCCCAG |
| | | CCTCTGT (SEQ ID NO. 167) |
| | G106F/L107K | G106F/L107K-F: TTCAAGTTCGGAAAGGGAGGCATCG |
| | | TGACTTG (SEQ ID NO. 168) |
| | | G106F/L107K-R: GAACTTGAAGCATCCATTGCCCCAG |
| | | CCTCTGT (SEQ ID NO. 169) |
| | G106F/L107R | G106F/L107R-F: TTCCGGTTCGGAAAGGGAGGCATCG |
| | | TGACTTG (SEQ ID NO. 170) |
| | | G106F/L107R-R: GAACCGGAAGCATCCATTGCCCCAG |
| | | CCTCTGT (SEQ ID NO. 171) |
| G106/F108 | G106F/F108W | G106F/F108W-F: TTCTTATGGGGAAAGGGAGGCATCGT |
| mutation | | GACTTG (SEQ ID NO. 172) |
| | | G106F/F108W-R: CCATAAGAAGCATCCATTGCCCCAGCC |
| | | TCTGT (SEQ ID NO. 173) |
| | G106L/F108W | G106L/F108W-F: CTGTTATGGGGAAAGGGAGGCATC |
| | | GTGACTTG (SEQ ID NO. 174) |
| | | G106L/F108W-R: CCATAACAGGCATCCATTGCCCCAG |
| | | CCTCTGT (SEQ ID NO. 175) |
| | G106F/F108H | G106F/F108H-F: TTCTTACACGGAAAGGGAGGCATC |
| | | GTGACTTG (SEQ ID NO. 176) |
| | | G106F/F108H-R: GTGTAAGAAGCATCCATTGCCCCAG |
| | | CCTCTGT (SEQ ID NO. 177) |
| | G106Y/F108W | G106Y/F108W-F: |
| | | TACTTATGGGGAAAGGGAGGCATCGTGACTTG (SEQ ID |
| | | NO. 178) |
| | | G106Y/F108W-R: CCATAAGTAGCATCCATTGCCCCAGCCT |
| | | CTGT (SEQ ID NO. 179) |
| | G106W/F108Y | G106W/F108Y-F: TGGTTATACGGAAAGGGAGGCATCGTG |
| | | ACTTG (SEQ ID NO. 180) |
| | | G106W/F108Y-R: GTATAACCAGCATCCATTGCCCCAGCCT |
| | | CTGT (SEQ ID NO. 181) |
| L107/F108 | L107E/F108W | L107E/F108W-F: GGTGAGTGGGGAAAGGGAGGCATCGT |
| mutation | | GACTTG (SEQ ID NO. 182) |
| | | L107E/F108W-R: CCACTCACCGCATCCATTGCCCCAGCC |
| | | TCTGT (SEQ ID NO. 183) |
| | L107K/F108W | L107K/F108W-F: GGTAAGTGGGGAAAGGGAGGCATCGTG |
| | | ACTTG (SEQ ID NO. 184) |
| | | L107K/F108W-R: CCACTTACCGCATCCATTGCCCCAGCCTC |
| | | TGT (SEQ ID NO. 185) |
| | L107R/F108W | L107R/F108W-F: GGTCGGTGGGGAAAGGGAGGCATCGTGA |
| | | CTTG (SEQ ID NO. 186) |
| | | L107R/F108W-R: CCACCGACCGCATCCATTGCCCCAGCCTC |
| | | TGT (SEQ ID NO. 187) |
| | L107D/F108W | L107D/F108W-F: GGTGACTGGGGAAAGGGAGGCATCGTGA |
| | | CTTG (SEQ ID NO. 188) |
| | | L107D/F108W-R: CCAGTCACCGCATCCATTGCCCCAGCCT |
| | | CTGT (SEQ ID NO. 189) |
| | L107K/F108Y | L107K/F108Y-F: GGTAAGTACGGAAAGGGAGGCATCGTGA |
| | | CTTG (SEQ ID NO. 190) |
| | | L107K/F108Y-R: GTACTTACCGCATCCATTGCCCCAGCCTC |
| | | TGT (SEQ ID NO. 191) |

293T cells were transfected with the wild-type plasmids pCAGGS-ZIKV-M/E-WT, pCAGGS-DENV2-M/E-WT and the mutant constructs in the above table, respectively. After 48 hours, the cells were harvested, digested into single cells, fixed and permeabilized, incubated with FL epitope-binding ADE antibodies Z6 and 2A10G6, and incubated with Goat Anti-Human (mouse) FITC secondary antibody. Finally, the positive proportion of samples was measured by flow cytometry. If the positive proportion was lower than 10% of the wild-type positive rate, it was considered as non-binding, and if 10%-50% of the wild-type positive rate, it was considered as weak binding. The results were shown in Table 14.

It can be seen from Table 14 that the above mutations could basically prevent the binding of the FL epitope representative ADE antibody, indicating that the vaccine prepared by the single point or synergistic mutation of G106, L107 and F108 represented by these mutations will reduce or avoid the production of the ADE antibodies that was induced by FL epitope, thereby avoiding the ADE effect on DENV after vaccine immunization.

TABLE 14
Results of DENV E protein mutants binding
to Z6 antibody and 2A10G6 antibody

FLE antibody

| Mutation site | Mutation form | Z6 | 2A10G6 |
|---|---|---|---|
| G106 mutation | G106L | — | — |
| | G106F | — | — |
| | G106W | — | — |
| | G106Y | — | — |
| | G106I | — | — |
| L107 mutation | L107E | — | — |
| | L107K | — | — |
| | L107R | — | — |
| | L107D | — | — |
| | L107T | — | — |
| F108 mutation | F108W | Weak binding | — |
| | F108H | — | — |
| | F108Y | — | — |
| | F108P | — | — |
| | F108A | — | — |
| G106/L107 | G106L/L107E | — | — |
| mutation | G106L/L107K | — | — |
| | G106F/L107E | — | — |
| | G106F/L107K | — | — |
| | G106F/L107R | — | — |
| G106/F108 | G106F/F108W | — | — |
| mutation | G106L/F108W | — | — |
| | G106F/F108H | — | — |
| | G106Y/F108W | — | — |
| | G106W/F108Y | — | — |
| L107/F108 | L107E/F108W | — | — |
| mutation | L107K/F108W | — | — |
| | L107R/F108W | — | — |
| | L107D/F108W | — | — |
| | L107K/F108Y | — | — |

Note:
— means no binding.

Finally, it should be noted that: the above examples were only used to illustrate the technical solutions of the present disclosure, but not to limit them. Although the present disclosure has been described in detail with reference to the foregoing examples, those of ordinary skill in the art should understand: modifications can still be made to the technical solutions described in the foregoing examples, or some technical features thereof can be equivalently replaced; and these modifications or replacements do not make the essence of the corresponding technical solutions depart from the spirit and scope of the technical solutions of the examples of the present disclosure.

### INDUSTRIAL APPLICABILITY

Examples in the present disclosure relates to a Zika/dengue vaccine and its application thereof.

The present application has obtained the epitope information of an antibody that causes ADE effect based on crystal structure analysis and other structural and functional analysis. The present disclosure provides antigens, in which some mutations are introduced into the E-protein FL fusion region of the Zika virus or dengue virus. All antigens with said mutations are unable to bind to antibodies causing ADE (FLE antigen). After immunization with the vaccine of the present disclosure acquired from the said antigens, production of FL epitope-induced antibodies can be prevented, thereby reducing or eliminating the ADE effect.

1. An antigen having an E protein FL fusion region of Zika virus or dengue virus, wherein the E protein FL fusion region comprises one of the following mutations:
(1) one or two of D98 and N103 site mutations in combination with a three-site mutation of G106, L107 and F108;
(2) one of G106, L107, and F108 site mutations or their combinations; and
(3) a single-site mutation of W101.

2. The antigen according to claim 1, wherein:
the mutation in the E protein FL fusion region is a five-site mutation of D98, N103, G106, L107 and F108; or,
the mutation in the E protein FL fusion region is a three-site mutation of G106, L107 and F108; or,
the mutation in the E protein FL fusion region is a double-site mutation of G106 and L107; or,
the mutation in the E protein FL fusion region is a double-site mutation of G106 and F108; or,
the mutation in the E protein FL fusion region is a double-site mutation of L107 and F108; or,
the mutation in the E protein FL fusion region is a single-site mutation of G106; or,
the mutation in the E protein FL fusion region is a single-site mutation of L107; or,
the mutation in the E protein FL fusion region is a single-site mutation of F108; or,
the mutation in the E protein FL fusion region is a single-site mutation of W101.

3. The antigen according to claim 1, wherein the mutation in the E protein FL fusion region is selected from any one or a combination of the following groups consisting of different mutation forms:

| mutation site | mutation form |
|---|---|
| five-site mutation of | D98N/N103T/G106F/L107E/F108W |
| D98/N103/G106/L107/F108 | D98N/N103T/G106F/L107K/F108W |
| | D98N/N103T/G106L/L107E/F108W |
| three-site mutation of | G106F/L107E/F108W |
| G106/L107/F108 | G106F/L107K/F108W |
| | G106L/L107E/F108W |
| double-site mutation | G106L/L107E |
| of G106/L107 | G106L/L107K |
| | G106F/L107E |
| | G106F/L107K |
| | G106F/L107R |
| double-site mutation | G106F/F108W |
| of G106/F108 | G106L/F108W |
| | G106F/F108H |
| | G106Y/F108W |
| | G106W/F108Y |
| double-site mutation | L107E/F108W |
| of L107/F108 | L107K/F108W |
| | L107R/F108W |
| | L107D/F108W |
| | L107K/F108Y |
| single-site mutation of G106 | G106L |
| | G106F |
| | G106W |
| | G106Y |
| | G106I |
| single-site mutation of L107 | L107E |
| | L107K |
| | L107R |
| | L107D |
| | L107T |
| single-site mutation of F108 | F108W |
| | F108H |
| | F108Y |
| | F108P |
| | F108A |
| single-site mutation of W101 | W101A |
| | W101R |
| | W101N |
| | W101D |
| | W101C |
| | W101Q |
| | W101E |
| | W101G |
| | W101H |
| | W101I |
| | W101L |
| | W101K |
| | W101M |
| | W101F |
| | W101P |
| | W101S |
| | W10IT |
| | W101Y |
| | W101V |

4. The antigen according to claim 1, wherein:
when the antigen comprises the E protein FL fusion region of Zika virus, the antigen further comprises a full sequence or a partial sequence of M protein of Zika virus; or,
when the antigen comprises the E protein FL fusion region of dengue virus, the antigen further comprises a full sequence or a partial sequence of M protein of dengue virus.

5. The antigen according to claim 1, wherein;
when the antigen comprises the E protein FL fusion region of Zika virus, the antigen further comprises a full sequence or a partial sequence of prM protein of Zika virus; or,
when the antigen comprises the E protein FL fusion region of dengue virus, the antigen further comprises a full-sequence or a partial sequence of prM protein of dengue virus.

6. The antigen according to claim 1, wherein:
when the antigen comprises the E protein FL fusion region of Zika virus, the antigen further comprises a full sequence or a partial sequence of E protein of Zika virus; or,
when the antigen comprises the E protein FL fusion region of dengue virus, the antigen further comprises a full sequence or a partial sequence of E protein of dengue virus.

7. An antigen binding epitope of the E protein FL fusion region of Zika virus, wherein the E protein FL fusion region of Zika viruses comprises the amino acid sequence of D98-R99-G100-W101-G102-N103-G104-C105-G106-L107-F108-G109, which comprises one of the following mutations:
(1) one or two of D98 and N103 site mutations in combination with a three-site mutation of G106, L107 and F108;
(2) one of G106, L107, and F108 site mutations or their combinations; and
(3) a single-site mutation of W101.

8. A Zika virus antigen, comprising the antigen binding epitope according to claim 7.

9. An antigen binding epitope of the E protein FL fusion region of dengue virus, wherein the E protein FL fusion region of dengue virus comprises the amino acid sequence of D98-R99-G100-W101-G102-N103-G104-C105-G106-L107-F108-G109, which comprises one of the following mutations:
(1) one or two of D98 and N103 site mutations in combination with a three-site mutation of G106, L107 and F108;
(2) one of G106, L107, and F108 site mutations or their combinations; and
(3) a single-site mutation of W101.

10. A dengue virus antigen comprising the antigen binding epitope according to claim 9.

11. An antibody that binds to the antigen according to claim 1.

12. A polynucleotide encoding the antigen according to any one of claim 1.

13. An expression cassette, recombinant vector, transgenic cell line, recombinant bacteria, adenovirus, lentivirus or viral particle, comprising the polynucleotide according to claim 12.

14. An mRNA encoding the antigen according to claim 1.

15. A vaccine comprising the antigen according to claim 1 as an active ingredient.

16. The vaccine according to claim 15, wherein the vaccine is one or more of an inactivated vaccine, an attenuated vaccine, a DNA vaccine, a mRNA vaccine, an adenovirus vaccine, other viral vector vaccines, a subunit vaccine or viral particles;
and/or, the vaccine further comprises a pharmaceutically or veterinarily acceptable vehicle, diluent, adjuvant or excipient.

17. A recombinant adenovirus vaccine, comprising a type 7 chimpanzee adenovirus, wherein the type 7 chimpanzee adenovirus is packaged with a recombinant vector;
wherein the recombinant vector comprises a backbone and a nucleic acid sequence of a target gene inserted to the backbone;
wherein the backbone is a replication-defective type 7 chimpanzee adenovirus vector; and
wherein the target gene comprises the polynucleotide according to claim 12.

18. A method for preventing and/or treating Zika virus or dengue virus infection in a subject, comprising administering a vaccine comprising the antigen according to claim 1 to the subject.

19. A method for detecting Zika virus or dengue virus infection in a subject, comprising using a detection agent or kit comprising the antigen according to claim 1.

20. A recombinant adenovirus vaccine, comprising a type 7 chimpanzee adenovirus, wherein the type 7 chimpanzee adenovirus is packaged with a recombinant vector;
wherein the recombinant vector comprises a backbone and a nucleic acid sequence of a target gene inserted to the backbone;
wherein the backbone is a replication-defective type 7 chimpanzee adenovirus vector; and
wherein the target gene comprises the mRNA according to claim 14.


SEQUENCE LISTING (191 sequences)

SEQ ID NO: 1 - protein, 504 aa, zika virus
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLVWLGLNTKNGSISLMCLALGGVLIFLSTAVSA

SEQ ID NO: 2 - DNA, 20 bp, Artificial Sequence
  Primer
ttttggcaaagaattcgccg

SEQ ID NO: 3 - DNA, 41 bp, Artificial Sequence
  Primer
gatctgctagctcgagtcaagcgctcacagctgtggacaga

SEQ ID NO: 4 - DNA, 55 bp, Artificial Sequence
  Primer
gtgtaagaggaccctggtgaacaggggctggggaacaggctgcttcgaatgggga

SEQ ID NO: 5 - DNA, 54 bp, Artificial Sequence
  Primer
gtgaacaggggctggggaacaggctgcttcgaatggggaaagggctccctggtg

SEQ ID NO: 6 - DNA, 55 bp, Artificial Sequence
  Primer
gtgtaagaggaccctggtgaacaggggctggggaacaggctgcttcaagtgggga

SEQ ID NO: 7 - DNA, 54 bp, Artificial Sequence
  Primer
gtgaacaggggctggggaacaggctgcttcaagtggggaaagggctccctggtg

SEQ ID NO: 8 - DNA, 55 bp, Artificial Sequence
  Primer
gtgtaagaggaccctggtgaacaggggctggggaacaggctgcctggaatgggga

SEQ ID NO: 9 - DNA, 54 bp, Artificial Sequence
  Primer
gtgaacaggggctggggaacaggctgcctggaatggggaaagggctccctggtg

SEQ ID NO: 10 - DNA, 37 bp, Artificial Sequence
  Primer
aaacgggccctctagagccaccatgggcaagaggagc

SEQ ID NO: 11 - DNA, 40 bp, Artificial Sequence
  Primer
tttaacttaagcttggtacctcaagcgctcacagctgtgg

SEQ ID NO: 12 - DNA, 30 bp, Artificial Sequence
  Primer
gtataactataacggtcctaaggtagcgaa

SEQ ID NO: 13 - DNA, 30 bp, Artificial Sequence
  Primer
tcattacctctttctccgcacccgacatag

SEQ ID NO: 14 - DNA, 20 bp, Artificial Sequence
  Primer
ccacacctctgccggcacac

SEQ ID NO: 15 - DNA, 20 bp, Artificial Sequence
  Primer
ttggctggcctatcaggttg

SEQ ID NO: 16 - DNA, 20 bp, Artificial Sequence
  Primer
cacctcggtttgagcactct

SEQ ID NO: 17 - protein, 24 aa, Japanese encephalitis virus
MGKRSAGSIMWLASLAVVIACAGA

SEQ ID NO: 18 - protein, 570 aa, Dengue virus
SVALVPHVGMGLETRTETWMSSEGAWKHAQRIETWILRHPGFTIMAAILAYTIGTTHFQRALIFILLTAVAPSMTMRCIGISNRDFVEGVSGGSWVDIVLEHGSCVTTMAKNKPTLDFELIKTEAKQPATLRKYCIEAKLTNTTTDSRCPTQGEPSLNEEQDKRFVCKHSMVDRGWGNGCGLFGKGGIVTCAMFTCKKNMKGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMIETTMRGAKRMAILGDTAWDFGSLGGVFTSIGKALHQVFGAIYGAAFSGVSWTMKILIGVIITWIGMNSRSTSLSVSLVLVGVVTLYLGVMVQA

SEQ ID NO: 19 - protein, 661 aa, Dengue virus
FHLTTRNGEPHMIVSRQEKGKSLLFKTEDGVNMCTLMAMDLGELCEDTITYKCPFLRQNEPEDIDCWCNSTSTWVTYGTCTTTGEHRREKRSVALVPHVGMGLETRTETWMSSEGAWKHAQRIETWILRHPGFTIMAAILAYTIGTTHFQRALIFILLTAVAPSMTMRCIGISNRDFVEGVSGGSWVDIVLEHGSCVTTMAKNKPTLDFELIKTEAKQPATLRKYCIEAKLTNTTTDSRCPTQGEPSLNEEQDKRFVCKHSMVDRGWGNGCGLFGKGGIVTCAMFTCKKNMKGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKKGSSIGQMIETTMRGAKRMAILGDTAWDFGSLGGVFTSIGKALHQVFGAIYGAAFSGVSWTMKILIGVIITWIGMNSRSTSLSVSLVLVGVVTLYLGVMVQA

SEQ ID NO: 20 - DNA, 31 bp, Artificial Sequence
  Primer for pshuttle-DV2-M/E-WT, pshuttle-DV2-prM/E-WT, and pshuttle-DV2-M/E-MutA/B/C
aaacgggccctctagagccaccatgctcaac

SEQ ID NO: 21 - DNA, 42 bp, Artificial Sequence
  Primer for pshuttle-DV2-M/E-WT, pshuttle-DV2-prM/E-WT, and pshuttle-DV2-M/E-MutA/B/C
tttaacttaagcttggtaccttaagcttgcaccatgactccc

SEQ ID NO: 22 - DNA, 73 bp, Artificial Sequence
  Primer for pshuttle-DV2-M/E-WT, pshuttle-DV2-prM/E-WT, and pshuttle-DV2-M/E-MutA/B/C
aaacgggccctctagagccaccatgctcaacattttaaacagaaggaggagaaccgccggaatgatcatcatg

SEQ ID NO: 23 - DNA, 71 bp, Artificial Sequence
  Primer for pshuttle-DV2-M/E-WT, pshuttle-DV2-prM/E-WT, and pshuttle-DV2-M/E-MutA/B/C
ggagaaccgccggaatgatcatcatgctgatccccaccgtgatggccagcgtggctctggtgccccatgtc

SEQ ID NO: 24 - DNA, 73 bp, Artificial Sequence; identical to SEQ ID NO: 22
  Primer for pshuttle-DV2-M/E-WT, pshuttle-DV2-prM/E-WT, and pshuttle-DV2-M/E-MutA/B/C

SEQ ID NO: 25 - DNA, 59 bp, Artificial Sequence
  Primer for pshuttle-DV2-M/E-WT, pshuttle-DV2-prM/E-WT, and pshuttle-DV2-M/E-MutA/B/C
gtgaacagaggctggggcacaggatgcttcgaatggggaaagggaggcatcgtgacttg

SEQ ID NO: 26 - DNA, 59 bp, Artificial Sequence
  Primer for pshuttle-DV2-M/E-WT, pshuttle-DV2-prM/E-WT, and pshuttle-DV2-M/E-MutA/B/C
tccccattcgaagcatcctgtgccccagcctctgttcaccatggagtgcttgcacacga

SEQ ID NO: 27 - DNA, 59 bp, Artificial Sequence
  Primer for pshuttle-DV2-M/E-WT, pshuttle-DV2-prM/E-WT, and pshuttle-DV2-M/E-MutA/B/C
gtgaacagaggctggggcacaggatgcttcaagtggggaaagggaggcatcgtgacttg

SEQ ID NO: 28 - DNA, 59 bp, Artificial Sequence
  Primer for pshuttle-DV2-M/E-WT, pshuttle-DV2-prM/E-WT, and pshuttle-DV2-M/E-MutA/B/C
tccccacttgaagcatcctgtgccccagcctctgttcaccatggagtgcttgcacacga

SEQ ID NO: 29 - DNA, 59 bp, Artificial Sequence
  Primer for pshuttle-DV2-M/E-WT, pshuttle-DV2-prM/E-WT, and pshuttle-DV2-M/E-MutA/B/C
gtgaacagaggatggggcacaggatgcctggaatggggcaagggctctttaatcacttg

SEQ ID NO: 30 - DNA, 59 bp, Artificial Sequence
  Primer for pshuttle-DV2-M/E-WT, pshuttle-DV2-prM/E-WT, and pshuttle-DV2-M/E-MutA/B/C
gccccattccaggcatcctgtgccccatcctctgttcacgaaggtcctacgacacacga

SEQ ID NO: 31 - protein, 579 aa, zika virus
AVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLVWLGLNTKNGSISLMCLALGGVLIFLSTAVSA

SEQ ID NO: 32 - DNA, 20 bp, Artificial Sequence
  Primer
ttttggcaaagaattcgccg

SEQ ID NO: 33 - DNA, 41 bp, Artificial Sequence; identical to SEQ ID NO: 3
  Primer for tryptophan mutation

SEQ ID NO: 34 - DNA, 36 bp, Artificial Sequence
  Primer for tryptophan mutation
cgcagccatttccggcgcccctgtccaccagggtcc

SEQ ID NO: 35 - DNA, 38 bp, Artificial Sequence
  Primer for tryptophan mutation
ggtggacaggggcgccggaaatggctgcggcctgtttg

SEQ ID NO: 36 - DNA, 36 bp, Artificial Sequence
  Primer for tryptophan mutation
cgcagccatttccccggcccctgtccaccagggtcc

SEQ ID NO: 37 - DNA, 38 bp, Artificial Sequence
  Primer for tryptophan mutation
ggtggacaggggccggggaaatggctgcggcctgtttg

SEQ ID NO: 38 - DNA, 36 bp, Artificial Sequence
  Primer for tryptophan mutation
cgcagccatttccgttgcccctgtccaccagggtcc

SEQ ID NO: 39 - DNA, 38 bp, Artificial Sequence
  Primer for tryptophan mutation
ggtggacaggggcaacggaaatggctgcggcctgtttg

SEQ ID NO: 40 - DNA, 36 bp, Artificial Sequence
  Primer for tryptophan mutation
cgcagccatttccgtcgcccctgtccaccagggtcc

SEQ ID NO: 41 - DNA, 38 bp, Artificial Sequence
  Primer for tryptophan mutation
ggtggacaggggcgacggaaatggctgcggcctgtttg

SEQ ID NO: 42 - DNA, 36 bp, Artificial Sequence
  Primer for tryptophan mutation
cgcagccatttccgcagcccctgtccaccagggtcc

SEQ ID NO: 43 - DNA, 38 bp, Artificial Sequence
  Primer for tryptophan mutation
ggtggacaggggctgcggaaatggctgcggcctgtttg

SEQ ID NO: 44 - DNA, 36 bp, Artificial Sequence
  Primer for tryptophan mutation
cgcagccatttccctggcccctgtccaccagggtcc

SEQ ID NO: 45 - DNA, 38 bp, Artificial Sequence
  Primer for tryptophan mutation
ggtggacaggggccagggaaatggctgcggcctgtttg

SEQ ID NO: 46 - DNA, 36 bp, Artificial Sequence
  Primer for tryptophan mutation
cgcagccatttccctcgcccctgtccaccagggtcc

SEQ ID NO: 47 - DNA, 38 bp, Artificial Sequence
  Primer for tryptophan mutation
ggtggacaggggcgagggaaatggctgcggcctgtttg

SEQ ID NO: 48 - DNA, 36 bp, Artificial Sequence
  Primer for tryptophan mutation
cgcagccatttccgccgcccctgtccaccagggtcc

SEQ ID NO: 49 - DNA, 38 bp, Artificial Sequence
  Primer for tryptophan mutation
ggtggacaggggcggcggaaatggctgcggcctgtttg

SEQ ID NO: 50 - DNA, 36 bp, Artificial Sequence
  Primer for tryptophan mutation
cgcagccatttccgtggcccctgtccaccagggtcc

SEQ ID NO: 51 - DNA, 38 bp, Artificial Sequence
  Primer for tryptophan mutation
ggtggacaggggccacggaaatggctgcggcctgtttg

SEQ ID NO: 52 - DNA, 36 bp, Artificial Sequence
  Primer for tryptophan mutation
cgcagccatttccgatgcccctgtccaccagggtcc

SEQ ID NO: 53 - DNA, 38 bp, Artificial Sequence
  Primer for tryptophan mutation
ggtggacaggggcatcggaaatggctgcggcctgtttg

SEQ ID NO: 54 - DNA, 36 bp, Artificial Sequence
  Primer for tryptophan mutation
cgcagccatttcccaggcccctgtccaccagggtcc

SEQ ID NO: 55 - DNA, 38 bp, Artificial Sequence
  Primer for tryptophan mutation
ggtggacaggggcctgggaaatggctgcggcctgtttg

SEQ ID NO: 56 - DNA, 36 bp, Artificial Sequence
  Primer for tryptophan mutation
cgcagccatttcccttgcccctgtccaccagggtcc

SEQ ID NO: 57 - DNA, 38 bp, Artificial Sequence
  Primer for tryptophan mutation
ggtggacaggggcaagggaaatggctgcggcctgtttg

SEQ ID NO: 58 - DNA, 36 bp, Artificial Sequence
  Primer for tryptophan mutation
cgcagccatttcccatgcccctgtccaccagggtcc

SEQ ID NO: 59 - DNA, 38 bp, Artificial Sequence
  Primer for tryptophan mutation
ggtggacaggggcatgggaaatggctgcggcctgtttg

SEQ ID NO: 60 - DNA, 36 bp, Artificial Sequence
  Primer for tryptophan mutation
cgcagccatttccgaagcccctgtccaccagggtcc

SEQ ID NO: 61 - DNA, 38 bp, Artificial Sequence
  Primer for tryptophan mutation
ggtggacaggggcttcggaaatggctgcggcctgtttg

SEQ ID NO: 62 - DNA, 36 bp, Artificial Sequence
  Primer for tryptophan mutation
cgcagccatttccggggcccctgtccaccagggtcc

SEQ ID NO: 63 - DNA, 38 bp, Artificial Sequence
  Primer for tryptophan mutation
ggtggacaggggccccggaaatggctgcggcctgtttg

SEQ ID NO: 64 - DNA, 36 bp, Artificial Sequence
  Primer for tryptophan mutation
cgcagccatttccgctgcccctgtccaccagggtcc

SEQ ID NO: 65 - DNA, 38 bp, Artificial Sequence
  Primer for tryptophan mutation
ggtggacaggggcagcggaaatggctgcggcctgtttg

SEQ ID NO: 66 - DNA, 36 bp, Artificial Sequence
  Primer for tryptophan mutation
cgcagccatttccggtgcccctgtccaccagggtcc

SEQ ID NO: 67 - DNA, 38 bp, Artificial Sequence
  Primer for tryptophan mutation
ggtggacaggggcaccggaaatggctgcggcctgtttg

SEQ ID NO: 68 - DNA, 36 bp, Artificial Sequence
  Primer for tryptophan mutation
cgcagccatttccgtagcccctgtccaccagggtcc

SEQ ID NO: 69 - DNA, 38 bp, Artificial Sequence
  Primer for tryptophan mutation
ggtggacaggggctacggaaatggctgcggcctgtttg

SEQ ID NO: 70 - DNA, 36 bp, Artificial Sequence
  Primer for tryptophan mutation
cgcagccatttcccacgcccctgtccaccagggtcc

SEQ ID NO: 71 - DNA, 38 bp, Artificial Sequence
  Primer for tryptophan mutation
ggtggacaggggcgtgggaaatggctgcggcctgtttg

SEQ ID NO: 72 - DNA, 32 bp, Artificial sequence
  Primer for sites mutation of G106, L107 and F108 of ZIKV E protein
ctgctgtttggaaagggctccctggtgacctg

SEQ ID NO: 73 - DNA, 32 bp, Artificial Sequence
  Primer for sites mutation of G106, L107 and F108 of ZIKV E protein
aaacagcaggcagccatttccccagcccctgt

SEQ ID NO: 74 - DNA, 32 bp, Artificial Sequence
  Primer for sites mutation of G106, L107 and F108 of ZIKV E protein
ttcctgtttggaaagggctccctggtgacctg

SEQ ID NO: 75 - DNA, 32 bp, Artificial Sequence
  Primer for sites mutation of G106, L107 and F108 of ZIKV E protein
aaacaggaagcagccatttccccagcccctgt

SEQ ID NO: 76 - DNA, 32 bp, Artificial Sequence
  Primer for sites mutation of G106, L107 and F108 of ZIKV E protein
tggctgtttggaaagggctccctggtgacctg

SEQ ID NO: 77 - DNA, 32 bp, Artificial Sequence
  Primer for sites mutation of G106, L107 and F108 of ZIKV E protein
aaacagccagcagccatttccccagcccctgt

SEQ ID NO: 78 - DNA, 32 bp, Artificial Sequence
  Primer for sites mutation of G106, L107 and F108 of ZIKV E protein
tacctgtttggaaagggctccctggtgacctg

SEQ ID NO: 79 - DNA, 32 bp, Artificial Sequence
  Primer for sites mutation of G106, L107 and F108 of ZIKV E protein
aaacaggtagcagccatttccccagcccctgt

SEQ ID NO: 80 - DNA, 32 bp, Artificial Sequence
  Primer for sites mutation of G106, L107 and F108 of ZIKV E protein
atcctgtttggaaagggctccctggtgacctg

SEQ ID NO: 81 - DNA, 32 bp, Artificial Sequence
  Primer for sites mutation of G106, L107 and F108 of ZIKV E protein
aaacaggatgcagccatttccccagcccctgt

SEQ ID NO: 82 - DNA, 32 bp, Artificial Sequence
  Primer for sites mutation of G106, L107 and F108 of ZIKV E protein
ggcgagtttggaaagggctccctggtgacctg

SEQ ID NO: 83 - DNA, 32 bp, Artificial Sequence
  Primer for sites mutation of G106, L107 and F108 of ZIKV E protein
aaactcgccgcagccatttccccagcccctgt

SEQ ID NO: 84 - DNA, 32 bp, Artificial Sequence
  Primer for sites mutation of G106, L107 and F108 of ZIKV E protein
ggcaagtttggaaagggctccctggtgacctg

SEQ ID NO: 85 - DNA, 32 bp, Artificial Sequence
  Primer for sites mutation of G106, L107 and F108 of ZIKV E protein
aaacttgccgcagccatttccccagcccctgt

SEQ ID NO: 86 - DNA, 32 bp, Artificial Sequence
  Primer for sites mutation of G106, L107 and F108 of ZIKV E protein
ggccggtttggaaagggctccctggtgacctg

SEQ ID NO: 87 - DNA, 32 bp, Artificial Sequence
  Primer for sites mutation of G106, L107 and F108 of ZIKV E protein
aaaccggccgcagccatttccccagcccctgt

SEQ ID NO: 88 - DNA, 32 bp, Artificial Sequence
  Primer for sites mutation of G106, L107 and F108 of ZIKV E protein
ggcgactttggaaagggctccctggtgacctg

SEQ ID NO: 89 - DNA, 32 bp, Artificial Sequence
  Primer for sites mutation of G106, L107 and F108 of ZIKV E protein
aaagtcgccgcagccatttccccagcccctgt

SEQ ID NO: 90 - DNA, 32 bp, Artificial Sequence
  Primer for sites mutation of G106, L107 and F108 of ZIKV E protein
ggcacctttggaaagggctccctggtgacctg

SEQ ID NO: 91 - DNA, 32 bp, Artificial Sequence
  Primer for sites mutation of G106, L107 and F108 of ZIKV E protein
aaaggtgccgcagccatttccccagcccctgt

SEQ ID NO: 92 - DNA, 32 bp, Artificial Sequence
  Primer for sites mutation of G106, L107 and F108 of ZIKV E protein
ggcctgtggggaaagggctccctggtgacctg

SEQ ID NO: 93 - DNA, 32 bp, Artificial Sequence
  Primer for sites mutation of G106, L107 and F108 of ZIKV E protein
ccacaggccgcagccatttccccagcccctgt

SEQ ID NO: 94 - DNA, 32 bp, Artificial Sequence
  Primer for sites mutation of G106, L107 and F108 of ZIKV E protein
ggcctgcacggaaagggctccctggtgacctg

SEQ ID NO: 95 - DNA, 32 bp, Artificial Sequence
  Primer for sites mutation of G106, L107 and F108 of ZIKV E protein
gtgcaggccgcagccatttccccagcccctgt

SEQ ID NO: 96 - DNA, 32 bp, Artificial Sequence
  Primer for sites mutation of G106, L107 and F108 of ZIKV E protein
ggcctgtacggaaagggctccctggtgacctg

SEQ ID NO: 97 - DNA, 32 bp, Artificial Sequence
  Primer for sites mutation of G106, L107 and F108 of ZIKV E protein
gtacaggccgcagccatttccccagcccctgt

SEQ ID NO: 98 - DNA, 32 bp, Artificial Sequence
  Primer for sites mutation of G106, L107 and F108 of ZIKV E protein
ggcctgcccggaaagggctccctggtgacctg

SEQ ID NO: 99 - DNA, 32 bp, Artificial Sequence
  Primer for sites mutation of G106, L107 and F108 of ZIKV E protein
gggcaggccgcagccatttccccagcccctgt

SEQ ID NO: 100 - DNA, 32 bp, Artificial Sequence
  Primer for sites mutation of G106, L107 and F108 of ZIKV E protein
ggcctggccggaaagggctccctggtgacctg

SEQ ID NO: 101 - DNA, 32 bp, Artificial Sequence
  Primer for sites mutation of G106, L107 and F108 of ZIKV E protein
ggccaggccgcagccatttccccagcccctgt

SEQ ID NO: 102 - DNA, 32 bp, Artificial Sequence
  Primer for sites mutation of G106, L107 and F108 of ZIKV E protein
ctggagtttggaaagggctccctggtgacctg

SEQ ID NO: 103 - DNA, 32 bp, Artificial Sequence
  Primer for sites mutation of G106, L107 and F108 of ZIKV E protein
aaactccaggcagccatttccccagcccctgt

SEQ ID NO: 104 - DNA, 32 bp, Artificial Sequence
  Primer for sites mutation of G106, L107 and F108 of ZIKV E protein
ctgaagtttggaaagggctccctggtgacctg

SEQ ID NO: 105 - DNA, 32 bp, Artificial Sequence
  Primer for sites mutation of G106, L107 and F108 of ZIKV E protein
aaacttcaggcagccatttccccagcccctgt

SEQ ID NO: 106 - DNA, 32 bp, Artificial Sequence
  Primer for sites mutation of G106, L107 and F108 of ZIKV E protein
ttcgagtttggaaagggctccctggtgacctg

SEQ ID NO: 107 - DNA, 32 bp, Artificial Sequence
  Primer for sites mutation of G106, L107 and F108 of ZIKV E protein
aaactcgaagcagccatttccccagcccctgt

SEQ ID NO: 108 - DNA, 32 bp, Artificial Sequence
  Primer for sites mutation of G106, L107 and F108 of ZIKV E protein
ttcaagtttggaaagggctccctggtgacctg

SEQ ID NO: 109 - DNA, 32 bp, Artificial sequence
  Primer for sites mutation of G106, L107 and F108 of ZIKV E protein
aaacttgaagcagccatttccccagcccctgt

SEQ ID NO: 110 - DNA, 32 bp, Artificial Sequence
  Primer for sites mutation of G106, L107 and F108 of ZIKV E protein
ttccggtttggaaagggctccctggtgacctg

SEQ ID NO: 111 - DNA, 32 bp, Artificial Sequence
  Primer for sites mutation of G106, L107 and F108 of ZIKV E protein
aaaccggaagcagccatttccccagcccctgt

SEQ ID NO: 112 - DNA, 32 bp, Artificial Sequence
  Primer for sites mutation of G106, L107 and F108 of ZIKV E protein
ttcctgtggggaaagggctccctggtgacctg

SEQ ID NO: 113 - DNA, 32 bp, Artificial Sequence
  Primer for sites mutation of G106, L107 and F108 of ZIKV E protein
ccacaggaagcagccatttccccagcccctgt

SEQ ID NO: 114 - DNA, 32 bp, Artificial Sequence
  Primer for sites mutation of G106, L107 and F108 of ZIKV E protein
ctgctgtggggaaagggctccctggtgacctg

SEQ ID NO: 115 - DNA, 32 bp, Artificial Sequence
  Primer for sites mutation of G106, L107 and F108 of ZIKV E protein
ccacagcaggcagccatttccccagcccctgt

SEQ ID NO: 116 - DNA, 32 bp, Artificial Sequence
  Primer for sites mutation of G106, L107 and F108 of ZIKV E protein
ttcctgcacggaaagggctccctggtgacctg

SEQ ID NO: 117 - DNA, 32 bp, Artificial Sequence
  Primer for sites mutation of G106, L107 and F108 of ZIKV E protein
gtgcaggaagcagccatttccccagcccctgt

SEQ ID NO: 118 - DNA, 32 bp, Artificial Sequence
  Primer for sites mutation of G106, L107 and F108 of ZIKV E protein
tacctgtggggaaagggctccctggtgacctg

SEQ ID NO: 119 - DNA, 32 bp, Artificial Sequence
  Primer for sites mutation of G106, L107 and F108 of ZIKV E protein
ccacaggtagcagccatttccccagcccctgt

SEQ ID NO: 120 - DNA, 32 bp, Artificial Sequence
  Primer for sites mutation of G106, L107 and F108 of ZIKV E protein
tggctgtacggaaagggctccctggtgacctg

SEQ ID NO: 121 - DNA, 32 bp, Artificial Sequence
  Primer for sites mutation of G106, L107 and F108 of ZIKV E protein
gtacagccagcagccatttccccagcccctgt

SEQ ID NO: 122 - DNA, 32 bp, Artificial Sequence
  Primer for sites mutation of G106, L107 and F108 of ZIKV E protein
ggcgagtggggaaagggctccctggtgacctg

SEQ ID NO: 123 - DNA, 32 bp, Artificial Sequence
  Primer for sites mutation of G106, L107 and F108 of ZIKV E protein
ccactcgccgcagccatttccccagcccctgt

SEQ ID NO: 124 - DNA, 32 bp, Artificial Sequence
  Primer for sites mutation of G106, L107 and F108 of ZIKV E protein
ggcaagtggggaaagggctccctggtgacctg

SEQ ID NO: 125 - DNA, 32 bp, Artificial Sequence
  Primer for sites mutation of G106, L107 and F108 of ZIKV E protein
ccacttgccgcagccatttccccagcccctgt

SEQ ID NO: 126 - DNA, 32 bp, Artificial Sequence
  Primer for sites mutation of G106, L107 and F108 of ZIKV E protein
ggccggtggggaaagggctccctggtgacctg

SEQ ID NO: 127 - DNA, 32 bp, Artificial Sequence
  Primer for sites mutation of G106, L107 and F108 of ZIKV E protein
ccaccggccgcagccatttccccagcccctgt

SEQ ID NO: 128 - DNA, 32 bp, Artificial Sequence
  Primer for sites mutation of G106, L107 and F108 of ZIKV E protein
ggcgactggggaaagggctccctggtgacctg

SEQ ID NO: 129 - DNA, 32 bp, Artificial Sequence
  Primer for sites mutation of G106, L107 and F108 of ZIKV E protein
ccagtcgccgcagccatttccccagcccctgt

SEQ ID NO: 130 - DNA, 32 bp, Artificial Sequence
  Primer for sites mutation of G106, L107 and F108 of ZIKV E protein
ggcaagtacggaaagggctccctggtgacctg

SEQ ID NO: 131 - DNA, 32 bp, Artificial Sequence
  Primer for sites mutation of G106, L107 and F108 of ZIKV E protein
gtacttgccgcagccatttccccagcccctgt

SEQ ID NO: 132 - DNA, 32 bp, Artificial Sequence
  Primer for mutation of G106, L107 and F108 sites of DENV E protein
ctgttattcggaaagggaggcatcgtgacttg

SEQ ID NO: 133 - DNA, 32 bp, Artificial Sequence
  Primer for mutation of G106, L107 and F108 sites of DENV E protein
gaataacaggcatccattgccccagcctctgt

SEQ ID NO: 134 - DNA, 32 bp, Artificial Sequence
  Primer for mutation of G106, L107 and F108 sites of DENV E protein
ttcttattcggaaagggaggcatcgtgacttg

SEQ ID NO: 135 - DNA, 32 bp, Artificial Sequence
  Primer for mutation of G106, L107 and F108 sites of DENV E protein
gaataagaagcatccattgccccagcctctgt

SEQ ID NO: 136 - DNA, 32 bp, Artificial Sequence
  Primer for mutation of G106, L107 and F108 sites of DENV E protein
tggttattcggaaagggaggcatcgtgacttg

SEQ ID NO: 137 - DNA, 32 bp, Artificial Sequence
  Primer for mutation of G106, L107 and F108 sites of DENV E protein
gaataaccagcatccattgccccagcctctgt

SEQ ID NO: 138 - DNA, 32 bp, Artificial Sequence
  Primer for mutation of G106, L107 and F108 sites of DENV E protein
tacttattcggaaagggaggcatcgtgacttg

SEQ ID NO: 139 - DNA, 32 bp, Artificial Sequence
  Primer for mutation of G106, L107 and F108 sites of DENV E protein
gaataagtagcatccattgccccagcctctgt

SEQ ID NO: 140 - DNA, 32 bp, Artificial Sequence
  Primer for mutation of G106, L107 and F108 sites of DENV E protein
atcttattcggaaagggaggcatcgtgacttg

SEQ ID NO: 141 - DNA, 32 bp, Artificial Sequence
  Primer for mutation of G106, L107 and F108 sites of DENV E protein
gaataagatgcatccattgccccagcctctgt

SEQ ID NO: 142 - DNA, 32 bp, Artificial Sequence
  Primer for mutation of G106, L107 and F108 sites of DENV E protein
ggtgagttcggaaagggaggcatcgtgacttg

SEQ ID NO: 143 - DNA, 32 bp, Artificial Sequence
  Primer for mutation of G106, L107 and F108 sites of DENV E protein
gaactcaccgcatccattgccccagcctctgt

SEQ ID NO: 144 - DNA, 32 bp, Artificial Sequence
  Primer for mutation of G106, L107 and F108 sites of DENV E protein
ggtaagttcggaaagggaggcatcgtgacttg

SEQ ID NO: 145 - DNA, 32 bp, Artificial Sequence
  Primer for mutation of G106, L107 and F108 sites of DENV E protein
gaacttaccgcatccattgccccagcctctgt

SEQ ID NO: 146 - DNA, 32 bp, Artificial Sequence
  Primer for mutation of G106, L107 and F108 sites of DENV E protein
ggtcggttcggaaagggaggcatcgtgacttg

SEQ ID NO: 147 - DNA, 32 bp, Artificial Sequence
  Primer for mutation of G106, L107 and F108 sites of DENV E protein
gaaccgaccgcatccattgccccagcctctgt

SEQ ID NO: 148 - DNA, 32 bp, Artificial Sequence
  Primer for mutation of G106, L107 and F108 sites of DENV E protein
ggtgacttcggaaagggaggcatcgtgacttg

SEQ ID NO: 149 - DNA, 32 bp, Artificial Sequence
  Primer for mutation of G106, L107 and F108 sites of DENV E protein
gaagtcaccgcatccattgccccagcctctgt

SEQ ID NO: 150 - DNA, 32 bp, Artificial Sequence
  Primer for mutation of G106, L107 and F108 sites of DENV E protein
ggtaccttcggaaagggaggcatcgtgacttg

SEQ ID NO: 151 - DNA, 32 bp, Artificial Sequence
  Primer for mutation of G106, L107 and F108 sites of DENV E protein
gaaggtaccgcatccattgccccagcctctgt

SEQ ID NO: 152 - DNA, 32 bp, Artificial Sequence
  Primer for mutation of G106, L107 and F108 sites of DENV E protein
ggtttatggggaaagggaggcatcgtgacttg

SEQ ID NO: 153 - DNA, 32 bp, Artificial Sequence
  Primer for mutation of G106, L107 and F108 sites of DENV E protein
ccataaaccgcatccattgccccagcctctgt

SEQ ID NO: 154 - DNA, 32 bp, Artificial Sequence
  Primer for mutation of G106, L107 and F108 sites of DENV E protein
ggtttacacggaaagggaggcatcgtgacttg

SEQ ID NO: 155 - DNA, 32 bp, Artificial Sequence
  Primer for mutation of G106, L107 and F108 sites of DENV E protein
gtgtaaaccgcatccattgccccagcctctgt

SEQ ID NO: 156 - DNA, 32 bp, Artificial Sequence
  Primer for mutation of G106, L107 and F108 sites of DENV E protein
ggtttatacggaaagggaggcatcgtgacttg

SEQ ID NO: 157 - DNA, 32 bp, Artificial Sequence
  Primer for mutation of G106, L107 and F108 sites of DENV E protein
gtataaaccgcatccattgccccagcctctgt

SEQ ID NO: 158 - DNA, 32 bp, Artificial Sequence
  Primer for mutation of G106, L107 and F108 sites of DENV E protein
ggtttacccggaaagggaggcatcgtgacttg

SEQ ID NO: 159 - DNA, 32 bp, Artificial Sequence
  Primer for mutation of G106, L107 and F108 sites of DENV E protein
gggtaaaccgcatccattgccccagcctctgt

SEQ ID NO: 160 - DNA, 32 bp, Artificial Sequence
  Primer for mutation of G106, L107 and F108 sites of DENV E protein
ggtttagccggaaagggaggcatcgtgacttg

SEQ ID NO: 161 - DNA, 32 bp, Artificial Sequence
  Primer for mutation of G106, L107 and F108 sites of DENV E protein
ggctaaaccgcatccattgccccagcctctgt

SEQ ID NO: 162 - DNA, 32 bp, Artificial Sequence
  Primer for mutation of G106, L107 and F108 sites of DENV E protein
ctggagttcggaaagggaggcatcgtgacttg

SEQ ID NO: 163 - DNA, 32 bp, Artificial Sequence
  Primer for mutation of G106, L107 and F108 sites of DENV E protein
gaactccaggcatccattgccccagcctctgt

SEQ ID NO: 164 - DNA, 32 bp, Artificial Sequence
  Primer for mutation of G106, L107 and F108 sites of DENV E protein
ctgaagttcggaaagggaggcatcgtgacttg

SEQ ID NO: 165 - DNA, 32 bp, Artificial sequence
  Primer for mutation of G106, L107 and F108 sites of DENV E protein
gaacttcaggcatccattgccccagcctctgt

SEQ ID NO: 166 - DNA, 32 bp, Artificial Sequence
  Primer for mutation of G106, L107 and F108 sites of DENV E protein
ttcgagttcggaaagggaggcatcgtgacttg

SEQ ID NO: 167 - DNA, 32 bp, Artificial Sequence
  Primer for mutation of G106, L107 and F108 sites of DENV E protein
gaactcgaagcatccattgccccagcctctgt

SEQ ID NO: 168 - DNA, 32 bp, Artificial Sequence
  Primer for mutation of G106, L107 and F108 sites of DENV E protein
ttcaagttcggaaagggaggcatcgtgacttg

SEQ ID NO: 169 - DNA, 32 bp, Artificial sequence
  Primer for mutation of G106, L107 and F108 sites of DENV E protein
gaacttgaagcatccattgccccagcctctgt

SEQ ID NO: 170 - DNA, 32 bp, Artificial Sequence
  Primer for mutation of G106, L107 and F108 sites of DENV E protein
ttccggttcggaaagggaggcatcgtgacttg

SEQ ID NO: 171 - DNA, 32 bp, Artificial Sequence
  Primer for mutation of G106, L107 and F108 sites of DENV E protein
gaaccggaagcatccattgccccagcctctgt

SEQ ID NO: 172 - DNA, 32 bp, Artificial Sequence
  Primer for mutation of G106, L107 and F108 sites of DENV E protein
ttcttatggggaaagggaggcatcgtgacttg

SEQ ID NO: 173 - DNA, 32 bp, Artificial Sequence
  Primer for mutation of G106, L107 and F108 sites of DENV E protein
ccataagaagcatccattgccccagcctctgt

SEQ ID NO: 174 - DNA, 32 bp, Artificial Sequence
  Primer for mutation of G106, L107 and F108 sites of DENV E protein
ctgttatggggaaagggaggcatcgtgacttg

SEQ ID NO: 175 - DNA, 32 bp, Artificial Sequence
  Primer for mutation of G106, L107 and F108 sites of DENV E protein
ccataacaggcatccattgccccagcctctgt

SEQ ID NO: 176 - DNA, 32 bp, Artificial Sequence
  Primer for mutation of G106, L107 and F108 sites of DENV E protein
ttcttacacggaaagggaggcatcgtgacttg

SEQ ID NO: 177 - DNA, 32 bp, Artificial Sequence
  Primer for mutation of G106, L107 and F108 sites of DENV E protein
gtgtaagaagcatccattgccccagcctctgt

SEQ ID NO: 178 - DNA, 32 bp, Artificial Sequence
  Primer for mutation of G106, L107 and F108 sites of DENV E protein
tacttatggggaaagggaggcatcgtgacttg

SEQ ID NO: 179 - DNA, 32 bp, Artificial Sequence
  Primer for mutation of G106, L107 and F108 sites of DENV E protein
ccataagtagcatccattgccccagcctctgt

SEQ ID NO: 180 - DNA, 32 bp, Artificial Sequence
  Primer for mutation of G106, L107 and F108 sites of DENV E protein
tggttatacggaaagggaggcatcgtgacttg

SEQ ID NO: 181 - DNA, 32 bp, Artificial Sequence
  Primer for mutation of G106, L107 and F108 sites of DENV E protein
gtataaccagcatccattgccccagcctctgt

SEQ ID NO: 182 - DNA, 32 bp, Artificial Sequence
  Primer for mutation of G106, L107 and F108 sites of DENV E protein
ggtgagtggggaaagggaggcatcgtgacttg

SEQ ID NO: 183 - DNA, 32 bp, Artificial Sequence
  Primer for mutation of G106, L107 and F108 sites of DENV E protein
ccactcaccgcatccattgccccagcctctgt

SEQ ID NO: 184 - DNA, 32 bp, Artificial Sequence
  Primer for mutation of G106, L107 and F108 sites of DENV E protein
ggtaagtggggaaagggaggcatcgtgacttg

SEQ ID NO: 185 - DNA, 32 bp, Artificial Sequence
  Primer for mutation of G106, L107 and F108 sites of DENV E protein
ccacttaccgcatccattgccccagcctctgt

SEQ ID NO: 186 - DNA, 32 bp, Artificial Sequence
  Primer for mutation of G106, L107 and F108 sites of DENV E protein
ggtcggtggggaaagggaggcatcgtgacttg

SEQ ID NO: 187 - DNA, 32 bp, Artificial Sequence
  Primer for mutation of G106, L107 and F108 sites of DENV E protein
ccaccgaccgcatccattgccccagcctctgt

SEQ ID NO: 188 - DNA, 32 bp, Artificial Sequence
  Primer for mutation of G106, L107 and F108 sites of DENV E protein
ggtgactggggaaagggaggcatcgtgacttg

SEQ ID NO: 189 - DNA, 32 bp, Artificial Sequence
  Primer for mutation of G106, L107 and F108 sites of DENV E protein
ccagtcaccgcatccattgccccagcctctgt

SEQ ID NO: 190 - DNA, 32 bp, Artificial Sequence
  Primer for mutation of G106, L107 and F108 sites of DENV E protein
ggtaagtacggaaagggaggcatcgtgacttg

SEQ ID NO: 191 - DNA, 32 bp, Artificial Sequence
  Primer for mutation of G106, L107 and F108 sites of DENV E protein
gtacttaccgcatccattgccccagcctctgt